(19) United States
(12) Patent Application Publication
(10) Pub. No.: US 2024/0352012 A1
(43) Pub. Date: Oct. 24, 2024

(54) PAN-CASPASE INHIBITORS

(71) Applicant: GEORGE MASON UNIVERSITY, Fairfax, VA (US)

(72) Inventors: Michael Girgis, Fairfax, VA (US); Mikell Paige, Fairfax, VA (US)

(73) Assignee: GEORGE MASON UNIVERSITY, Fairfax, VA (US)

(21) Appl. No.: 18/632,761

(22) Filed: Apr. 11, 2024

Related U.S. Application Data
(60) Provisional application No. 63/495,443, filed on Apr. 11, 2023.

(51) Int. Cl. C07D 471/04; A61K 31/4745; A61K 31/5025; A61K 31/5377

(52) U.S. Cl. CPC C07D 471/04; A61K 31/4745; A61K 31/5025; A61K 31/5377

(57) ABSTRACT

Embodiments relate to novel pan caspase inhibitors and method of manufacture of the same. Additionally, embodiments provide pharmaceutical compositions using novel pan caspase inhibitors, and methods for the use thereof as caspase inhibitors and for the treatment of disorders caused by excessive apoptotic activity.

### RELATED APPLICATION

This application claims priority from U.S. provisional application No. 63/495,443, filed on Apr. 11, 2023, titled “Pan-Caspase Inhibitors”, which is incorporated herein by reference in its entirety.

### BACKGROUND OF INVENTION

Programmed cell death, also known as apoptosis, is a natural process that occurs in all cells as a means of removing damaged or unwanted cells. However, in some disease conditions, such as liver disease and neurodegenerative diseases, apoptosis can become dysregulated and contribute to tissue damage and organ dysfunction.

Pan-caspase inhibitors are a class of drugs that target multiple caspases, which are enzymes involved in the apoptotic pathway, and can prevent cells from undergoing apoptosis. In this paper, we will discuss the therapeutic benefits of pan-caspase inhibitors, the current ones available in the market, the major risks and problems associated with their use, and the potential for future improvements.

The therapeutic benefits of pan-caspase inhibitors are related to their ability to prevent apoptosis in disease conditions where excessive apoptosis contributes to tissue damage and dysfunction. Some examples of such conditions are: liver disease, cancer, neurodegenerative disorder, viral infection, and ischemia-reperfusion injury. where excessive apoptosis of liver cells (hepatocytes) can lead to liver failure.

In the past few years, there have been several attempts to synthesize pan-caspase inhibitors includes several small molecule inhibitors that have been developed and tested in various preclinical and clinical studies. These inhibitors can broadly be classified into two types: irreversible and reversible.

Irreversible inhibitors, such as IDN-6556 (Emricasan), bind covalently to the active site of caspases and prevent their activity. Reversible inhibitors, such as Q-VD-OPh, bind non-covalently to the active site of caspases and prevent their activity. Other pan-caspase inhibitors include VX-166, AT-406 and AZD-5582, which are currently being tested in clinical trials.

Ac-DEVD-CHO and Q-VD-Oph (FIG. 1) are not drug-like and have poor bioavailability because of their peptide nature. Furthermore, they are relatively large molecules and polar in nature which makes it difficult to penetrate the blood brain barrier which minimize their utility in the medical field.

In a more recent study Sunesis used a modified version of the tethering technology to produce a more potent, less reversible molecule that was found to be effective against caspase 8 as well as caspase 3. See, FIG. 2 for the chemical structure of the compound described by Erlanson et al.

In an effort from Merck company to reduce the peptidic nature of these molecules Several compounds were synthesized using solid-phase synthetic methods. FIG. 6 shows the chemical structure of the compounds as described by Isabel et al. In an effort from Merck company to reduce the peptidic nature of these molecules Several compounds were synthesized using solid-phase synthetic methods. FIG. 3 shows the chemical structure of the compounds as described by Isabel et al. Lastly, Merck has patented and published the chemical synthesis and the structure of the tricyclic inhibitor called Granzyme B. It was discovered that cytotoxic cells secrete granules containing 5 Granzyme B and this molecule transfers across the cellular membrane to activate caspase 3 and 7 to induce apoptosis. In a later publication Willoughby et al. described a potent selective Granzyme B inhibitor that can block CTL mediated apoptosis (see, Panel C of FIG. 3)

Although structure-based design of inhibitors for caspase 3 and/or caspase 7 has successfully produced potent peptide and Peptidomimetic compounds, these inhibitors typically have pharmacokinetic and pharmacodynamics challenges. In addition, the complex structure and unusual physicochemical properties of these compounds have limited their application in medical treatments and their utility in various pharmaceutical preparations. The development of nonpeptidic inhibitors that can overcome these limitations was always a mystery to scientists who are interested in this field.

One of the major needs for new pan-caspase inhibitors is to improve their specificity and reduce off-targets effects. Caspases are involved in several cellular processes, such as inflammation and immune response, and their inhibition can have unintended consequences. For example, inhibition of caspase-1 can lead to the accumulation of inflammasome components and increased inflammation, while inhibition of caspase-8 can impair the immune response to viral infections. Therefore, it is important to design inhibitors that target specific caspases or have minimal off-target effects.

Another need for new pan-caspase inhibitors is to improve their pharmacokinetic properties, such as bioavailability and half-life. Many pan-caspase inhibitors have poor solubility and stability, which limits their effectiveness in vivo. Furthermore, the rapid clearance of these inhibitors from the body can require frequent dosing, leading to potential toxicity and reduced patient compliance. Therefore, there is a need to develop inhibitors that have better pharmacokinetic profiles and can be administered less frequently.

Another problem with pan-caspase inhibitors is their potential toxicity. Caspases play a role in several physiological processes, such as cell differentiation and immune response, and their inhibition can have unintended consequences. For example, inhibition of caspase-3 can impair muscle regeneration, while inhibition of caspase-9 can impair dendritic cell maturation. Therefore, it is important to evaluate the safety of pan-caspases inhibitors in preclinical and clinical studies and design inhibitors that have minimal toxicity.

### SUMMARY OF INVENTION

While there was a continuous effort to produce potent peptide inhibitors for caspase 3 and or caspase 7. poor cell permeability, low metabolic stability and the unfavorable physicochemical profile of these peptides hindered their use for therapeutic purposes and opened the door for a new adventure in designing nonpeptide small molecules like those mentioned in this research study. Despite the fact that the literature showed the importance of a variety of substituents at position 8 of the main scaffold, the literature did not show the role of any substitution at position 2 of the scaffold ring system. There were a few attempts by scientists to try to synthesize derivatives of the molecular scaffold with a variety of substitutions; however, there was no clear understanding of the structure activity relationships of these molecules.

An embodiment relates to a very efficient method of synthesizing the molecular scaffold by cyclizing the produced β-ketoester with a proper isotin ring or derivatized isotin to form the quinoline ring using the Pfitzinger technique.

In an embodiment, molecules were synthesized, purified, characterized and evaluated biologically for activity and potency against caspase 3 enzyme and caspase 7 enzyme respectively.

In an embodiment, computational molecular modeling and docking studies were performed to contemplate in the intermolecular interactions and thoroughly analyze the SAR of these inhibitors.

In an embodiment, one or more inhibitors were shown to be effective against both pillars of the apoptosis machinery (caspase 3 and caspase 7) which is very unique and provides further endorsement to the inhibitory activity and potency especially on the cellular level. This was confirmed by the computational docking studies and the in-vitro bioassays performed.

In an embodiment, a compound having Formula I (1,4-dioxo-5-methyl-2,3,4-trihydro-1H-pyridazino[4,5-c]quinoline, with the structure as shown below is provided:

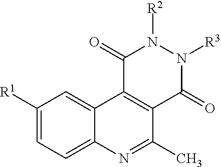

In an embodiment, Formula I is mainly modified at positions 2, 3 and/or 9. For example: In an embodiment, “R¹”, “R²”, and “R³” in Formula I is independently represent substitution on the indicated structure with one or more Hydrogen; Hydroxyl; Alkyl group; Alkoxy group; Acyloxy group; Alkenyl group; Alkynyl group; Aromatic group; Heterocyclic group; or any salt, isomer, ester, or derivative thereof. Alkyl group could be either of straight Alkyl group, branched Alkyl group; cyclic Alkyl group; heterocyclic aliphatic group; or substituted alkyl group.

In an embodiment, “R¹”, “R²”, and “R³” in formula I independently represent substitution on the indicated structure with an aromatic group. The aromatic group could be Monocyclic aromatic group, Polycyclic aromatic group or any substituted moiety thereof, or any salt, isomer, ester, or derivative thereof.

In an embodiment, “R¹” in formula I represents substitution with one or more of Amino groups, such as Nitrogen-containing groups, which can be primary (NH₂), secondary (NHR), or tertiary (NR₂), with R being any alkyl or aryl group, Hydrogen; Halogen; Nitro group; Sulfhydryl group; Carboxyl group; Cyano group; Sulfonyl group (SO₂R): whereas R may be an alkyl or aryl group. In an embodiment, R group on Sulfonyl group (SO₂R) may be a heterocyclic aliphatic or heterocyclic aromatic structures; or any salt, isomer, ester, or derivative thereof, for the modulation of caspase activity.

In an embodiment, “R¹” in formula I represents substitution with a heterocyclic aliphatic group. The heterocylic aliphatic compound could be selected from a piperidine, pyrrolidine, morpholine, imidazolidine, pyrane, or any substituted moiety thereof or any salt, isomer, ester, or derivative thereof.

In an embodiment, wherein “R²” and “R³” in formula I independently represent substitution is an aromatic group comprise at least one five or six membered ring or any substituted moiety thereof; or any salt, isomer, ester, or derivative thereof.

In an embodiment, wherein “R²” and “R³” in formula I independently represent substitution having an aromatic group. The aromatic group could be selected from a benzene ring, a pyridine ring, a quinoline ring, or any substituted moiety thereof or any salt, isomer, ester, or derivative thereof.

In an embodiment, the disclosure provides a compound having Formula II, wherein structure of the Formula II is:

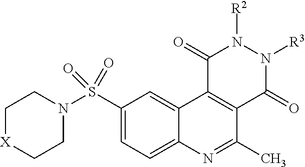

wherein “R²” and “R³” comprise at least one of Hydrogen; Hydroxyl; Alkyl group; Alkoxy group; Acyloxy group; Alkenyl group; Alkynyl group; Aromatic group; Heterocyclic group; and “X” is a (0), (—CH₂—), (—O—), or (CH₃N—) or any salt, isomer, ester, or derivative, or any allosteric replacement thereof.

In an embodiment, “R²”, and “R³” in formula II independently represent substitution on the indicated structure with an aromatic group. The aromatic group could be Monocyclic aromatic group, Polycyclic aromatic group or any substituted moiety thereof, or any salt, isomer, ester, or derivative thereof.

In an embodiment, wherein “R²” and “R³” in formula II independently represent substitution is an aromatic group comprise at least one five or six membered ring or any substituted moiety thereof; or any salt, isomer, ester, or derivative thereof.

In an embodiment, wherein “R²” and “R³” in formula II independently represent substitution having an aromatic group. The aromatic group could be selected from a benzene ring, a pyridine ring, a quinoline ring, or any substituted moiety thereof or any salt, isomer, ester, or derivative thereof.

In an embodiment, the disclosure provides a compound having Formula III, wherein structure of the Formula III is:

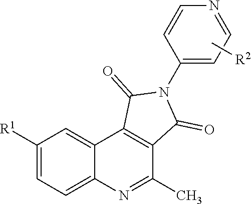

In an embodiment, “R¹” and “R²”, in Formula III is independently represent substitution on the indicated structure with one or more Hydrogen; Hydroxyl; Alkyl group; Alkoxy group; Acyloxy group; Alkenyl group; Alkynyl group; fused aromatic group; Halogen; Nitro group; Sulfhydryl group; Carboxyl group; Cyano group; fused heteroaromatic, fused aliphatic or fused heteroaliphatic moiety; or any salt, isomer, ester, or derivative thereof.

In an embodiment, “R²”, is an aromatic group. The aromatic group could be such as benzene ring, pyridine ring, quinoline ring. In some embodiments aromatic group could be halogenated aromatic groups such as without limitation a fluorinated benzene ring, a fluorinated pyridine ring, a fluorinated quinoline ring, or any substituted moiety thereof; or any salt, isomer, ester, or derivative thereof.

In an embodiment, “R¹” in Formula III is the sulfonamide group having formula (—SO₂R). In an embodiment, R in the (—SO₂R) could be a heterocyclic aliphatic group or heterocyclic aromatic structures. In an embodiment, “R¹” in Formula III comprises the sulfonamide group comprising a cyclic amino moiety. In an embodiment, “R¹” in Formula III comprises the sulfonamide group comprising a heterocyclic aliphatic amine.

In an embodiment, the heterocyclic aliphatic group could be selected without limitation from a piperidine, pyrrolidine, morpholine, imidazolidine, pyrane, or any substituted moiety thereof, or any salt, isomer, ester, or derivative thereof, for the modulation of caspase activity.

In an embodiment, a pharmaceutical composition comprising any one of the compounds, according to claim 1, for the diagnosis and treatment of pathological conditions that involve upregulation of caspase activity in peripheral blood cells or other body cells individually or in any combination with other known drugs and a pharmaceutically acceptable vehicle.

In an embodiment, a diagnostic kit comprising any one of the compounds in claim 1, for the MRI or PET scan imaging and treatment of pathological conditions that involve upregulation of caspase activity individually or in any combination with other known drugs and a pharmaceutically acceptable vehicle.

In an embodiment, a method for the synthesis of any of compound, according to claim 1, wherein The synthesis involve converting Isotin or a derivative thereof to the dicarboxylic acid quinoline adduct, by adding methyl acetoacetate in a strong alkaline medium, then acidification. The produced quinoline adduct is refluxed in acetic anhydride to form the anhydride. Finally, the anhydride is converted to the imide by refluxing with the amine of the phenylhydrazine or any derivatives thereof.

In an embodiment, the compound has IC₅₀ against any of the caspase enzymes is less than 10 μM. wherein the compound has an inhibitory activity against caspase 1, caspase 3 and caspase 7.

In an embodiment, a compound, wherein the inhibitory potential of the compound is more against caspase 7 compared to caspase 1 and caspase 3.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The accompanying drawings, which are included to provide further understanding of the present invention disclosed in the present disclosure and are incorporated in and constitute a part of this specification, illustrate aspects of the present invention and together with the description serve to explain the principles of the present invention. In the drawings:

FIG. 1 shows a chemical structure of the peptide Ac-DEVD-CHO (panel A) and chemical structure of the peptide Q-VD-Oph (panel B).

FIG. 2 shows a molecular structure of the compound described by Erlanson et al.

FIG. 3 shows the general formula of the compound described by Isabel et a (panel A and B) and by Willoughby et al. (panel C).

FIG. 4 shows a possible mode of action of caspase 3 inhibitors through the nucleophilic attack on the carbonyl groups of the phthalimide like ring.

FIG. 5 shows the IC₅₀ plot of compounds MO10, MO13, MO14, and MO15 against caspase 1.

FIG. 6 shows IC₅₀ plot of compounds MO6A MO6B, PE6A and PE6B against caspase 1.

FIG. 7 shows dynamic charts of the ranges of IC₅₀ against caspase 3 within 95% confidence limit.

FIG. 8 shows intermolecular interactions of compound MO6A with the binding pocket residues of caspase 3 enzyme.

FIG. 9 shows intermolecular interactions of compound MO6B with the binding pocket residues of caspase 3 enzyme.

FIG. 10 shows intermolecular interactions of compound PE6A with the binding pocket residues of caspase 3 enzyme.

FIG. 11 shows intermolecular interactions of compound PE6B with the binding pocket residues of caspase 3 enzyme.

FIG. 12 shows dynamic charts of the ranges of IC₅₀ against caspase 7 within 95% confidence limit.

FIG. 13 shows intermolecular interactions of compound MO6A with the binding pocket residues of caspase 7 enzyme.

FIG. 14 shows intermolecular interactions of compound MO6B with the binding pocket residues of caspase 7 enzyme.

FIG. 15 shows intermolecular interactions of compound PE6A with the binding pocket residues of caspase 7 enzyme.

FIG. 16 shows intermolecular interactions of compound PE6B with the binding pocket residues of caspase 7 enzyme.

FIG. 17 shows active caspase 8 in T cells, B cells and Monocytes (panel A) and caspase 8 in monocytes −/+MO6 (panel B).

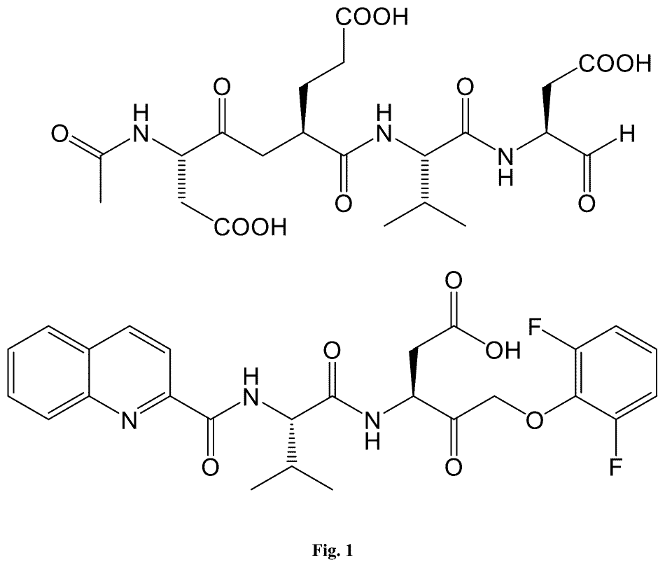

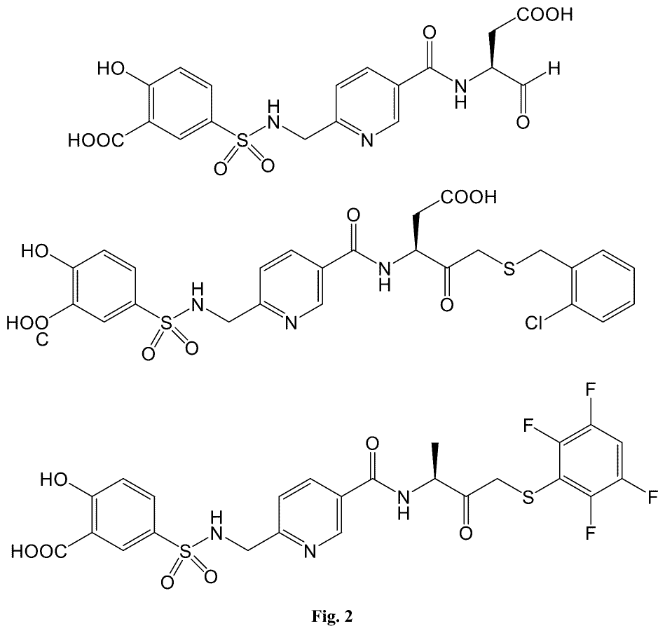

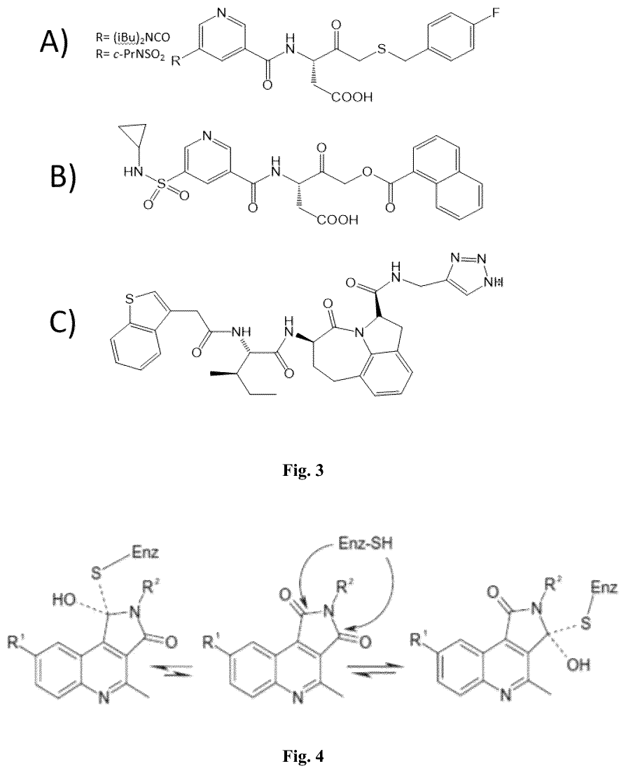

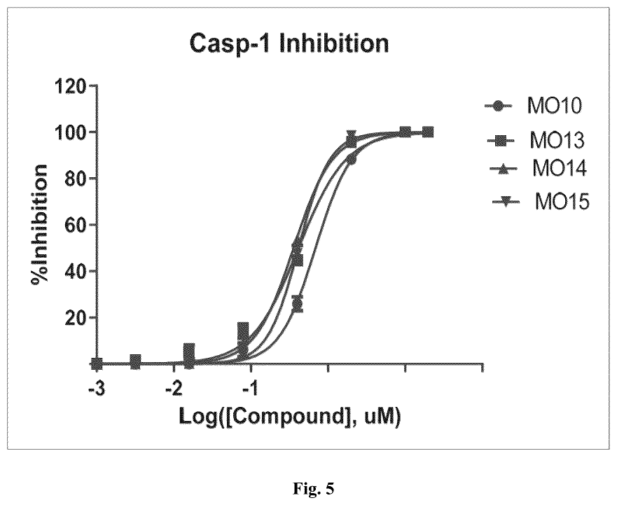

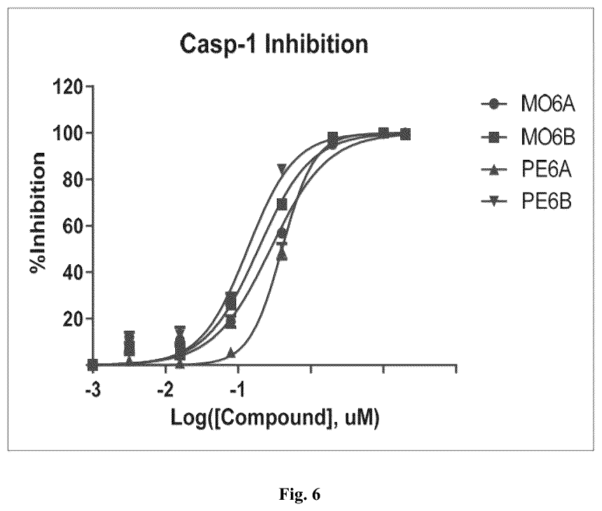

### DETAILED DESCRIPTION

### Definitions and General Techniques

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denotes the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

No element, act, or instruction used herein should be construed as critical or essential unless explicitly described as such. Also, as used herein, the articles “a” and “an” are intended to include items, and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

The methods and techniques of the present invention are generally performed according to conventional methods well known in the art and as described in various general and more specific references that are cited and discussed throughout the present specification unless otherwise indicated. The nomenclatures used in connection with, and the procedures and techniques of embodiments herein, and other related fields described herein are those well-known and commonly used in the art.

The recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

The following terms and phrases, unless otherwise indicated, shall be understood to have the following meanings.

The term, “Alkyl” refers to a mono-radical branched or unbranched saturated hydrocarbon chain preferably having from 1 to 22 carbon atoms and to cycloalkyl groups having one or more rings having 3 to 22 carbon atoms. Short alkyl groups are those having 1 to 6 carbon atoms including methyl, ethyl, propyl, butyl, pentyl and hexyl groups, including all isomers thereof. Long alkyl groups are those having 8-22 carbon atoms and preferably those having 12-22 carbon atoms as well as those having 12-20 and those having 16-18 carbon atoms.

The term, “Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, aryl-O—, and alkynyl-O—, where alkenyl, cycloalkyl, cycloalkenyl, and alkynyl are as defined herein.

The term, “Acyloxy” refers to the groups alkyl-C(O)O—, substituted alkyl-C(O)O, cycloalkyl-C(O)O—, substituted cycloalkyl-C(O)O—, aryl-C(O)O—, heteroaryl-C(O)O—, and heterocyclyl-C(O)O wherein alkyl, substituted alkyl, cycloalkyl, substituted cycloalkyl, aryl, heteroaryl, and heterocyclyl are as defined herein.

The term, “Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers. The term ‘alkenyl’ includes substituted alkenyl. The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, SO-alkyl, SO substituted alkyl, SO-aryl, SO-heteroaryl, SO₂-alkyl, —SO₂-substituted alkyl, —SO₂-aryl and —SO₂-heteroaryl.

The term, “Alkynyl group” refers to refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH). The term ‘alkenyl’ includes substituted alkynyl group. The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, SO-alkyl, SO substituted alkyl, SO-aryl, SO-heteroaryl, SO₂-alkyl, —SO₂-substituted alkyl, SO₂-aryl, and —SO₂-heteroaryl.

The term, “Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, SO-alkyl, SO-substituted alkyl, SO-aryl, SO-heteroaryl, SO₂-alkyl, SO₂-substituted alkyl, SO₂-aryl, SO₂-heteroaryl and trihalomethyl.

The term, “Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, SO-alkyl, SO-substituted alkyl, SO-aryl, SO-heteroaryl, SO₂-alkyl, SO₂-substituted alkyl, SO₂-aryl and SO₂-heteroaryl, and trihalomethyl, S(O)₂R; wherein each occurrence of R independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additionally, it will be appreciated, that any two adjacent groups taken together may represent a 4, 5, 6, or 7-membered cyclic, substituted or unsubstituted aliphatic or heteroaliphatic moiety. Additional examples of generally applicable substituents are illustrated by the specific embodiments shown in the Examples that are described herein.

The term, “Heteroaralkyl” refers to the groups -alkylene-heteroaryl where alkylene and heteroaryl are defined herein. This term includes, by way of example, pyridylmethyl, pyridylethyl, indolylmethyl, and the like.

The term, “Heteroaryloxy” refers to O-heteroaryl.

The term, “Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms.

These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, S(O)—, or SO₂ moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, SO-alkyl, SO-substituted alkyl, SO-aryl, SO-heteroaryl, —SO₂-alkyl, SO₂-substituted alkyl, —SO₂-aryl, SO₂-heteroaryl, and fused heterocycle.

The term, “Carboxyl,” “carboxy” or “carboxylate” refers to CO₂H or salts thereof.

The term, “Carboxyl ester” or “carboxy ester” or the terms “carboxyalkyl” or “carboxylalkyl” refers to the groups C(O)O-alkyl, —C(O)O-substituted alkyl, C(O)O— alkenyl, —C(O)O-substituted alkenyl, C(O)O-alkynyl, —C(O)O-substituted alkynyl, C(O)O-aryl, C(O)O-substituted aryl, C(O)O-cycloalkyl, —C(O)O-substituted cycloalkyl, C(O)O— cycloalkenyl, C(O)O-substituted cycloalkenyl, C(O)O-heteroaryl, —C(O)O-substituted heteroaryl, C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term, “(Carboxyl ester)oxy” or “carbonate” refers to the groups O—C(O)O-alkyl, —O—C(O)O-substituted alkyl, O—C(O)O-alkenyl, —O—C(O)O-substituted alkenyl, —O—C(O)O— alkynyl, O—C(O)O-substituted alkynyl, O—C(O)O-aryl, O—C(O)O— substituted aryl, O—C(O)O cycloalkyl, —O—C(O)O-substituted cycloalkyl, O—C(O)O— cycloalkenyl, O—C(O)O-substituted cycloalkenyl, O—C(O)O-heteroaryl, O—C(O)O— substituted heteroaryl, —O—C(O)O-heterocyclic, and O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term, “Cyano” or “nitrile” refers to the group CN.

The term, “Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

Substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, SO-alkyl, SO-substituted alkyl, SO-aryl, SO-heteroaryl, —SO₂-alkyl, —SO₂-substituted alkyl, —SO₂-aryl and —SO₂-heteroaryl.

The term, “Cycloalkenyl” refers to non-aromatic cyclic alkyl groups of from 3 to 10 carbon atoms having single or multiple rings and having at least one double bond and preferably from 1 to 2 double bonds.

The term, “Substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, SO-alkyl, SO-substituted alkyl, SO-aryl, SO-heteroaryl, —SO₂-alkyl, —SO₂-substituted alkyl, —SO₂-aryl and —SO₂-heteroaryl.

The term, “Cycloalkynyl” refers to non-aromatic cycloalkyl groups of from 5 to 10 carbon atoms having single or multiple rings and having at least one triple bond.

The term, “Cycloalkoxy” refers to O-cycloalkyl.

The term, “Cycloalkenyloxy” refers to O-cycloalkenyl.

The term, “Halo” or “halogen” refers to an atom selected from fluorine, chlorine, bromine and iodine.

The term, “Hydroxy” or “hydroxyl” refers to the group OH.

The term, “Nitro” refers to the group NO₂.

The term, “Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cycloalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂, and 4-methylphenyl-SO₂—

The term, “Sulfhydryl group” refers to the —SH group.

The term, “Substituted” refers to any of the above groups (e.g., alkyl, alkylene, alkenylene, alkynylene, heteroalkylene, heteroalkenylene, heteroalkynylene, alkoxy, alkyl ether, alkoxy alkyl ether, heteroalkyl, heteroalkoxy, phosphoalkyl, phosphoalkyl ether, thiophosphoalkyl, thiophosphoalkyl ether, carbocyclic, cycloalkyl, aryl, heterocyclic and/or heteroaryl), wherein at least one hydrogen atom (e.g., one, two, three, or all hydrogen atoms) is a non-hydrogen atom (e.g., but not limited to: F, Cl, Br, and I) halogen atoms such as; oxygen atoms in groups such as hydroxyl groups, alkoxy groups, and ester groups; sulfur atoms in groups such as thiol groups, thioalkyl groups, sulfone groups, sulfonyl groups, and sulfoxide groups; amides, alkylamines, dialkylamines, arylamines, alkylarylamines, diarylamines, N-oxides, imides, and enamines; trialkylsilyl groups, dialkylarylsilyl groups, alkyldiarylsilyl and silicon atoms in groups such as triarylsilyl groups; and other heteroatoms in a variety of other groups). “Substituted” also means that one or more hydrogen atoms are replaced by heteroatoms (e.g., oxygen in oxo, carbonyl, carboxyl, and ester groups; and imines, oximes, hydrazones, and nitriles).

The term, “isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of this invention. Such compounds are useful, for example, as analytical tools, as probes in biological assays, or as therapeutic agents in accordance with the present invention.

Where a particular enantiomer is preferred, it may, in some embodiments be provided substantially free of the corresponding enantiomer, and may also be referred to as “optically enriched.” “Optically-enriched,” as used herein, means that the compound is made up of a significantly greater proportion of one enantiomer. In certain embodiments the compound is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, IN 1972).

The term, “pharmaceutically acceptable salt” means a salt which is acceptable for administration to a patient, such as a mammal (salts with counterions having acceptable mammalian safety for a given dosage regime). It further refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. Pharmaceutical acceptable salts can be derived from pharmaceutically acceptable inorganic or organic bases and from pharmaceutically acceptable inorganic or organic acids. Pharmaceutically acceptable salt could be derived from a variety of organic and inorganic counter ions well known in the art. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

The term, “Pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

The term, “Pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety that is susceptible to removal in vivo yielding the parent molecule as the pharmacologically active species. An example of a pro-drug is an ester which is cleaved in vivo to yield a compound of interest. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

The term, “Caspase” as used herein (cysteine-aspartic proteases, cysteine aspartases or cysteine-dependent aspartate-directed proteases) are a family of protease enzymes playing essential roles in programmed cell death. Caspase are secreted in form of procaspase that based on stimulation covert to caspase results in the generation of the active “executioner” caspase form that subsequently catalyzes the hydrolysis of a multitude of protein substrates. Caspase includes without limitation caspase-1, caspase-2, caspase-3, caspase-4, caspase-5, caspase-6, caspase-7, caspase-8, caspase-9, caspase-10, caspase-11, caspase-12, caspase-13, and/or caspase-14.

The term, “IC₅₀” by definition is half maximal inhibitory concentration (IC₅₀) which is a measure of the effectiveness of a substance in blocking a specific biological or biochemical function. This quantitative measure indicates how much of a particular drug or other substance (inhibitor) is needed to inhibit a given biological process by half. The values are typically expressed as molar concentration. A lower IC₅₀ reflects a higher potency and a higher IC₅₀ value is correlated to a lower potency.

The term, “Modulating”, “modulator”, inhibit” or “inhibiting” refers to suppressing, reducing, decreasing, or substantially eliminating the biological activity of a polypeptide of a caspase. In an embodiment, a compound can affect a procaspase. In another embodiment, the compound can affect executioner caspase. In an embodiment, compound of the disclosure can act on a specific caspase such as caspase-1, caspase-2, caspase-3, caspase-4, caspase-5, caspase-6, caspase-7, caspase-8, caspase-9, caspase-10, caspase-11, caspase-12, caspase-13, and/or caspase-14. In an embodiment, the compounds of the disclosure can inhibit a large set of different types of caspases independent of each other. In an embodiment, the compounds can inhibit or modulate the caspase such as but not limited to caspase 1, caspase 3 and caspase 7. As will be understood by those skilled in the art upon, inhibition of caspase of a cell can be achieved in a number of manners. In some embodiments the inhibition can be achieved by competitive blockage of the active site of the enzyme by compounds of the disclosure. Inhibitors are believed to act like competitive inhibitors because they associate with the same cysteine residue responsible for the caspase activity. In other words, the molecules compete with the substrate on the active site. This leads to blocking of the active site of the enzyme and mitigates the enzyme substrate binding reactivity. In some embodiment, one or more compounds of the disclosure can affect the polypeptide chain of the caspase.

The term, “Pan-caspase inhibitors”, “anti-caspase” or “caspase inhibitors” or similar terms are interchangeably used through the specification. They are defined as a compound that act on one or more of the known caspases and are pursued for their ability to treat diseases such as autoimmune disorders, neurogenerative disorder, viral infection, cancer, etc.

“Differential inhibitory action” refers to a varied degree of suppressing, reducing, decreasing, or substantially eliminating the biological activity two or more different types of caspases by an inhibitor. Such caspases could be selected from caspase-1, caspase-2, caspase-3, caspase-4, caspase-5, caspase-6, caspase-7, caspase-8, caspase-9, caspase-10, caspase-11, caspase-12, caspase-13, and/or caspase-14. For example: in one embodiment, inhibitory action of the compounds of the disclosure is more enhanced for caspase 7 compared to caspase 3 and/or 1. The differential inhibitory action of an inhibitor could vary without limitation by 50%, 60%, 70%, 80%, 90%, 100% or more between 2 or more caspases. In some cases, the differential inhibitory action of a compound could vary without limitation by 2 times, 3 times, 5 times, 7 times, 10 times, 15 times, 20 times, or more among different caspases.

“Subject” is intended to include human and non-human animals. Non-human animals include all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include human patients in need of suppression of caspase activity or undesired apoptosis.

“Treatment”, “treat” and “treating” encompasses alleviation, cure or prevention of at least one symptom or other aspect of a disorder, disease, illness or other condition (collectively referred to herein as a “condition”), or reduction of severity of the condition, and the like. A composition of the invention need not affect a complete cure, or eradicate every symptom or manifestation of a disease, to constitute a viable therapeutic agent. As is recognized in the pertinent field, drugs employed as therapeutic agents may reduce the severity of a given disease state, but need not abolish every manifestation of the disease to be regarded as useful therapeutic agents. Beneficial or desired clinical results include, but are not limited to, alleviation of symptoms, diminishment of extent of disease, stabilization (i.e., not worsening) of disease, delay or slowing of disease progression, amelioration or palliation of the disease state, remission (whether partial or total, whether detectable or undetectable) and prevention of relapse or recurrence of disease. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient.

“Treatment” can also mean prolonging survival as compared to expected survival if not receiving treatment. In one embodiment, an indication that a therapeutically effective amount of a composition has been administered to the patient is a sustained improvement over baseline of an indicator that reflects the severity of a particular disorder.

### Embodiments

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

It should be noted herein that any feature or component described in association with a specific embodiment may be used and implemented with any other embodiment unless clearly indicated otherwise.

### Compounds

In an embodiment, a compound having Formula I (1,4-dioxo-5-methyl-2,3,4-trihydro-1H-pyridazino[4,5-c]quinoline, (and. Pharmaceutically Acceptable Derivatives thereof) with the structure as shown below is provided:

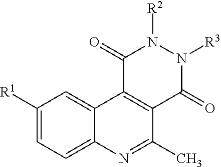

In an embodiment, Formula I is mainly modified at positions 2, 3 and/or 9. For example: In an embodiment, “R¹”, “R²”, and “R³” in Formula I is independently represent substitution on the indicated structure with one or more Hydrogen; Hydroxyl; Alkyl group; Alkoxy group; Acyloxy group; Alkenyl group; Alkynyl group; Aromatic group; Heterocyclic group; or any salt, isomer, ester, or derivative thereof. Alkyl group could be either of straight Alkyl group, branched Alkyl group; cyclic Alkyl group; heterocyclic aliphatic group; or substituted alkyl group.

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, CH₂-Cyclopropyl, allyl, n-butyl, sec-butyl, isobutyl, tert-butyl, cyclobutyl, CH₂-cyclobutyl, n-pentyl, sec-pentyl, isopentyl, tert-pentyl, cyclopentyl, CH₂-cyclopentyl-n, hexyl, sec-hexyl, cyclohexyl, CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargy1), 1-propynyl and the like.

In an embodiment, “R¹”, “R²”, and “R³” in one or more compounds of the disclosure could be substituted or unsubstituted.

In an embodiment, substituted moiety; or any salt, isomer, ester, or derivative thereof is capable of modulating caspase enzyme.

In an embodiment, “R¹”, “R²”, and “R³” in formula I independently represent substitution on the indicated structure with an aromatic group. The aromatic group could be Monocyclic aromatic group, Polycyclic aromatic group or any substituted moiety thereof, or any salt, isomer, ester, or derivative thereof.

In an embodiment, “R¹” in formula I represents substitution with one or more of Amino groups, such as Nitrogen-containing groups, which can be primary (NH₂), secondary (NHR), or tertiary (NR₂), with R being any alkyl or aryl group, Hydrogen; Halogen; Nitro group; Sulfhydryl group; Carboxyl group; Cyano group; Sulfonyl group (SO₂R): whereas R may be an alkyl or aryl group. In an embodiment, R group on Sulfonyl group (SO₂R) may be a heterocyclic aliphatic or heterocyclic aromatic structures; or any salt, isomer, ester, or derivative thereof, for the modulation of caspase activity.

In an embodiment, “R¹” in formula I represents substitution with a heterocyclic aliphatic group. The heterocylic aliphatic compound could be selected without limitation from a piperidine, pyrrolidine, morpholine, imidazolidine, pyrane, or any substituted moiety thereof or any salt, isomer, ester, or derivative thereof.

In an embodiment, wherein “R²” and “R³” in formula I independently represent substitution is an aromatic group having either five or six membered ring or any substituted moiety thereof; or any salt, isomer, ester, or derivative thereof.

In an embodiment, wherein “R²” and “R³” in formula I independently represent substitution having an aromatic group. The aromatic group could be for example without limitation, a benzene ring, a pyridine ring, a quinoline ring, or any substituted moiety thereof or any salt, isomer, ester, or derivative thereof.

In an embodiment, the disclosure provides a compound having Formula II, wherein structure of the Formula II is:

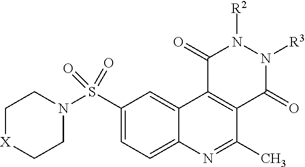

wherein “R²” and “R³” comprise at least one of Hydrogen; Hydroxyl; Alkyl group; Alkoxy group; Acyloxy group; Alkenyl group; Alkynyl group; Aromatic group; Heterocyclic group; and “X” is a (0), (—CH₂—), (—O—), or (CH₃N—) or any salt, isomer, ester, or derivative or any allosteric replacement thereof.

In an embodiment, “R²”, and “R³” in formula II independently represent substitution on the indicated structure with an aromatic group. The aromatic group could be Monocyclic aromatic group, Polycyclic aromatic group or any substituted moiety thereof, or any salt, isomer, ester, or derivative thereof.

In an embodiment, wherein “R²” and “R³” in formula II independently represent substitution is an aromatic group comprise at least one five or six membered ring or any substituted moiety thereof; or any salt, isomer, ester, or derivative thereof.

In an embodiment, wherein “R²” and “R³” in formula II independently represent substitution having an aromatic group. The aromatic group could be selected from a benzene ring, a pyridine ring, a quinoline ring, or any substituted moiety thereof or any salt, isomer, ester, or derivative thereof.

In an embodiment, the disclosure provides a compound having Formula III (and. Pharmaceutically Acceptable Derivatives thereof), wherein structure of the Formula III is:

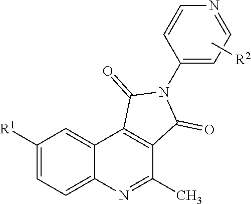

In an embodiment, “R¹” and “R²”, in Formula III is independently represent substitution on the indicated structure with one or more Hydrogen; Hydroxyl; Alkyl group; Alkoxy group; Acyloxy group; Alkenyl group; Alkynyl group; fused aromatic group; Halogen; Nitro group; Sulfhydryl group; Carboxyl group; Cyano group; fused heteroaromatic, fused aliphatic or fused heteroaliphatic moiety; or any salt, isomer, ester, or derivative thereof.

In an embodiment, “R²”, is an aromatic group. The aromatic group could be such as benzene ring, pyridine ring, quinoline ring. In some embodiment aromatic group could be halogenated aromatic groups such as without limitation a fluorinated benzene ring, a fluorinated pyridine ring, a fluorinated quinoline ring, or any substituted moiety thereof; or any salt, isomer, ester, or derivative thereof.

In an embodiment, “R¹” in Formula III is the sulfonamide group having formula (—SO₂R). In an embodiment, R in the (—SO₂R) could be a heterocyclic aliphatic group or heterocyclic aromatic structures. In an embodiment, “R¹” in Formula III comprises the sulfonamide group comprising a cyclic amino moiety. In an embodiment, “R¹” in Formula III comprises the sulfonamide group comprising a heterocyclic aliphatic amine.

In an embodiment, the heterocyclic aliphatic group could be selected without limitation from a piperidine, pyrrolidine, morpholine, imidazolidine, pyrane, or any substituted moiety thereof, or any salt, isomer, ester, or derivative thereof, for the modulation of caspase activity.

In an embodiment, the disclosure provides a compound having Formula IV (1,3-dioxo-4-methyl-2,3dihydro-1H-pyrrolo[3,4-c]quinoline) (and. Pharmaceutically Acceptable Derivatives thereof) that are mainly modified at positions 2 and 8, wherein structure of the Formula IV is:

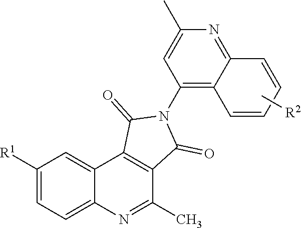

In an embodiment, “R¹” and “R²”, in Formula IV is independently represent substitution on the indicated structure with one or more Hydrogen; Hydroxyl; Alkyl group; Alkoxy group; Acyloxy group; Alkenyl group; Alkynyl group; fused aromatic group; Halogen; Nitro group; Sulfhydryl group; Carboxyl group; Cyano group; fused heteroaromatic, fused aliphatic or fused heteroaliphatic moiety; or any salt, isomer, ester, or derivative thereof.

In an embodiment, “R²”, is an aromatic group. The aromatic group could be such as benzene ring, pyridine ring, quinoline ring. In some embodiment aromatic group could be halogenated aromatic groups such as without limitation a fluorinated benzene ring, a fluorinated pyridine ring, a fluorinated quinoline ring, or any substituted moiety thereof; or any salt, isomer, ester, or derivative thereof.

In an embodiment, “R¹” in Formula IV is the sulfonamide group having formula (—SO₂R). In an embodiment, R in the (—SO₂R) could be a heterocyclic aliphatic group or heterocyclic aromatic structures. In an embodiment, “R¹” in Formula III comprises the sulfonamide group comprising a cyclic amino moiety. In an embodiment, “R¹” in Formula IV comprises the sulfonamide group comprising a heterocyclic aliphatic amine.

In an embodiment, the heterocyclic aliphatic group could be selected without limitation from a piperidine, pyrrolidine, morpholine, imidazolidine, pyrane, or any substituted moiety thereof, or any salt, isomer, ester, or derivative thereof, for the modulation of caspase activity.

In an embodiment, R¹ in Formula IV can be a sulfonamide group with a cyclic amino moiety chosen from pyrrolidine, piperidinyl, or morpholinyl. Also, R² is H, CH₃, allylic, normal alkyl, branched alkyl, unsaturated alkyl, cyclic alkyl, a halogen, —SO₃H, —COOH, —NO₂, —CHO, —CN, —NH₂, —NR₂, or r any derivative or salt thereof.

In an embodiment, some of the foregoing compounds can exist in various isomeric forms, e.g., stereoisomers and/or diastereomers. Furthermore, certain compounds, as described herein may have one or more double bonds that can exist as either the Z or E isomer, unless otherwise indicated. The invention additionally encompasses the compounds as individual isomers (e.g., as either the R or S enantiomer) substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives, as well as compositions comprising one or more compounds in combination with one or more additional therapeutic agents.

In an embodiment, compounds of the invention may be prepared by crystallization of compound of formula (I) under different conditions and may exist as various polymorphs of compound of general formula (I) forming part of this invention. For example, using different solvents commonly used or their mixtures for recrystallization; crystallizations at different temperatures; various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram or such other techniques. Thus, the present invention encompasses inventive compounds, their derivatives, their tautomeric forms, their stereoisomers, their polymorphs, their pharmaceutically acceptable salts their pharmaceutically acceptable solvates and pharmaceutically acceptable compositions containing them.

Combinations of substituents and variables envisioned by this invention are preferably those that result in the formation of stable compounds useful in the treatment, for example of caspase-mediated disorders, as described generally above. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

### Method of Synthesis

An embodiment relates to nonpeptide small molecules synthesized using a modified Pfitzinger's technique like the synthetic method described earlier in Kravchenko et al.

In an embodiment, compounds of the disclosure could be synthesized using solid isotin or isotin derivatives.

An embodiment relates to nonpeptide small molecules synthesized using a modified Pfitzinger's technique like the synthetic method described earlier in Kravchenko et al.

In an embodiment, compounds of the disclosure could be synthesized using solid isotin or isotin derivatives.

In an embodiment, formula 1,3-dioxo-4-methyl-2,3-dihydro-1H-pyrrolo[3,4-c]quinoline that are mainly modified at positions 2 and 8, and Formula I that are mainly modified at positions 2, 3 and 9 with a variety of substituents to target and modulate the caspase cascade, wherein, X, R¹, and R², are as described in the disclosure.

### Biological Activity

According to the present invention, the inventive compounds may be assayed in any of the available assays known in the art for identifying compounds having caspase inhibitory activity.

For example, the assay may be cellular or non-cellular, in vivo or in vitro, high- or low-throughput format, etc.

In certain exemplary embodiments, compounds of this invention were assayed for their ability to inhibit caspases 1, 3 and 7.

In an embodiment, compounds of this invention which are of particular interest include those which:
exhibit the ability to act as inhibitors of caspases;
exhibit the ability to act as inhibitors of apoptotic caspases;
exhibit the ability to act as inhibitors of caspases 1;
exhibit the ability to act as inhibitors of caspases 3;
exhibit the ability to act as inhibitors of caspases 7;
exhibit the ability to act as inhibitors of caspases 1, caspases 3 and/or caspases 7;
are useful in therapeutic applications related to caspase-mediated diseases.

In certain embodiments, one or more compounds of the invention are selective for Caspase 3 inhibitors.

In certain embodiments, one or more compounds of the invention are selective for Caspase 7 inhibitors.

In certain embodiments, one or more compounds of the invention are selective for Caspase 1 inhibitors.

In certain embodiments, one or more compounds of the invention are broad spectrum inhibitors of Caspases. IC₅₀ of one or more compounds of the disclosure against various caspases are less than 100 μM, 10 μM, 1 μM, 0.1 μM, 100 nM, 10 nM, 1 nM, 0.1 nM, 100 μmol, 10 μmol, or less.

In certain exemplary embodiments, one or more compounds of the invention have IC₅₀ value for caspase 1 is less than 100 μM, 10 μM, 1 μM, 0.1 μM, 100 nM, 10 nM, 1 nM, 0.1 nM, 100 μmol, 10 μmol, or less.

In an embodiment, IC₅₀ of one or more compounds of the disclosure is less than 100 μM, 10 μM, 1 μM, 0.1 μM, 100 nM, 10 nM, 1 nM, 0.1 nM, 100 μmol, 10 μmol, or less for Caspase 3.

In an embodiment, IC₅₀ of compounds of the disclosure is less than 100 μM, 10 μM, 1 μM, 0.1 μM, 100 nM, 10 nM, 1 nM, 0.1 nM, 100 μmol, 10 μmol, or less for caspase7.

In an embodiment, IC₅₀ of compounds of the disclosure is less than 100 μM, 10 μM, 1 μM, 0.1 μM, 100 nM, 10 nM, 1 nM, 0.1 nM, 100 μmol, 10 μmol, or less for any type of caspases such as but not limited Caspase 1, 3 and 7.

In an embodiment, one or more compounds shows differential inhibitory action against various caspases, such as caspase 1, caspase 2, caspase 3, caspase 7, caspase 8, caspase 6, caspase 9 etc. In an embodiment, one or more compounds show more inhibitor action towards caspase 7 compared to caspase 3 and/or 1. In certain embodiment, the differential inhibitory action could be about 2 times, 3, 4, 5, 6, 7, 8, 10, 15, 20 or 25 times more compared to caspase3 and/or 1.

In an embodiment, one or more compounds of the disclosure has a broader spectra of anti-caspase activity. A broad-spectrum anti-caspase activity signifies that a compound of the disclosure can act as an inhibitor for than one type of caspase, for example, a compound has anti-caspase 3 and anti-caspase 7 activities independently.

In an embodiment, one or more compounds of the disclosure attains a reaction equilibrium with a caspase enzyme in about 5 mins, 10 mins, 20 mins, 30 mins, 40 mins, 60 mins or more.

In an embodiment, the inhibitors showed differentially higher inhibitory potency against caspase 7 compared to caspase 3 and caspase 1.

### Method of Treatment

In certain embodiments, one or more compounds as described herein exhibit activity generally as inhibitors of caspases. In an embodiment, one or more compounds of the invention demonstrate activity as inhibitors of apoptotic caspases and thus, in another aspect, the invention further provides a method for treating caspase-mediated disorders (such as those associated with abnormally high apoptosis) including, but not limited to, stroke, traumatic, brain injury, spinal cord injury, meningitis, Alzheimer's disease, Parkinson's disease, Huntington's disease, Kennedy's disease, prion disease, multiple sclerosis, spinal muscular atrophy, myocardial infarction, congestive heart failure and various other forms of acute and chronic heart disease, atherosclerosis, aging, burns, organ transplant rejection, graft versus host disease, hepatitis-B, -C, -G, various forms of liver disease including acute alcoholic hepatitis, yellow fever, dengue fever, Japanese encephalitis, glomerulonephritis, renal disease, H. pylori-associated gastric and duodenal ulcer disease, HIV infection, tuberculosis, alopecia, diabetes, sepsis, Shigellosis, uveitis, inflammatory peritonitis, pancreatitis, erythematosus, scleroderma, chronic thyroiditis, Graves' disease, autoimmune gastritis, autoimmune hemolytic anemia, autoimmune neutropenia, thrombocytopenia, HIV-related encephalitis, myasthenia gravis, small bowel ischemia in disease or post-surgery, psoriasis, atopic dermatitis, myelodysplastic syndrome, acute and chronic myelogenous leukemia, metastatic melanoma, Kaposi's sarcoma, and Wiscott-Aldrich syndrome.

In an embodiment, one or more compounds of disclosure can treat diseases characterized by cell death or inflammation in acute and/or chronic conditions. For instance, in neurodegenerative conditions like amyotrophic lateral sclerosis (ALS) and following traumatic brain injury (TBI), myocardial infarction, stroke, in ophthalmic diseases such as retinal detachment and diabetic retinopathy, pulmonary disorders like idiopathic, pulmonary fibrosis (IPF), in sickle cell disease which involves upregulated caspase4 activity in platelets, intensifying during pain crises, and viral infections diseases like COVID-19, influenza and HIV, or post viral syndrome and cardiovascular diseases like Microvascular Antiphospholipid Syndrome (MAPS).

In certain embodiments, the method involves the administration of a therapeutically effective amount of the compound or a pharmaceutically acceptable derivative thereof to a subject (including, but not limited to a human or animal) in need of it. In certain embodiments, a pharmaceutical composition comprising an inventive compound (or pharmaceutically acceptable derivative thereof), a carrier or diluent and optionally an additional therapeutic agent is provided.

### Pharmaceutical Composition

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof.

A “pharmaceutically acceptable carrier,” as used herein refers to pharmaceutical excipients, for example, pharmaceutically, physiologically, acceptable organic, or inorganic carrier substances suitable for enteral or parenteral application which do not deleteriously react with the extract. Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the compounds of the invention.

Provided compounds can be administered alone or can be coadministered to a patient along with one or more other drugs. Coadministration is meant to include simultaneous or sequential administration of the compounds individually or in combination (more than one compound). In some embodiments, the preparations are combined with other active substances (e.g. to reduce metabolic degradation).

Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, as described above, in another aspect of the invention, a method for the treatment of caspase-mediated disorders is provided comprising administering a therapeutically effective amount of a compound of formula (I) as described herein, to a subject in need thereof. In another embodiment, a method for the treatment of caspase-mediated disorders is provided comprising administering a therapeutically effective amount of a compound of formula (II) as described herein, to a subject in need thereof. In an embodiment, a method for the treatment of caspase-mediated disorders is provided comprising administering a therapeutically effective amount of a compound of formula (III) as described herein, to a subject in need thereof. In an embodiment, a method for the treatment of caspase-mediated disorders is provided comprising administering a therapeutically effective amount of a compound of formula (IV) as described herein, to a subject in need thereof.

In certain embodiments, the method is utilized for the treatment of disorders mediated by apoptotic caspases, and in particular, those disorders resulting from an overactive apoptotic response. It will be appreciated that the compounds and compositions, according to the method of the present invention, may be administered using any amount and any route of administration effective for the treatment of caspase-mediated disorders (and including the treatment of disorders mediated by apoptotic caspases). For example, in certain exemplary embodiments, compounds of the invention are useful as inhibitors of apoptosis and thus can be used for the treatment of disorders including, but not limited to, cancer, immune disorders, HIV infection, and Alzheimer's disease, neurodegenerative disorder to name a few.

The term, therapeutically effective amount” and “effective amount” as used herein, refers to a sufficient amount of agent to cause a detectable decrease in the severity of the disease or in caspase activity and/or cell apoptosis, as measured by any of the assays described in the examples herein. The exact amount required will vary from subject to subject, depending on the species, age, and general condition of the subject, the severity of the infection, the particular therapeutic agent, its mode of administration, and the like. The compounds of the invention are preferably formulated in dosage unit form for ease of administration and uniformity of dosage.

The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts (see, for example, Goodman and Gilman's, “The Pharmacological Basis of Therapeutics”, Tenth Edition, A. Gilman, J. Hardman and L. Limbird, eds., McGraw-Hill Press, 155-173, 2001, which is incorporated herein by reference in its entirety).

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anti-apoptotic agent, for example), or they may achieve different effects (e.g., control of any adverse effects).

For example, other therapies or anticancer agents that may be used in combination with the inventive compounds of the present invention include surgery, radiotherapy (in but a few examples, γ-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

In certain embodiments, the pharmaceutical compositions of the present invention further comprises one or more additional therapeutically active ingredients (e.g., chemotherapeutic and/or palliative). For purposes of the invention, the term “Palliative” refers to treatment that is focused on the relief of symptoms of a disease and/or side effects of a therapeutic regimen, but is not curative. For example, palliative treatment encompasses painkillers, antinausea medications and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

### Kits

In other embodiments, the present invention relates to a kit for conveniently and effectively carrying out the methods in accordance with the present invention. In some embodiment, the kit could be used for diagnostic purpose to detect apoptosis activity and/or caspase activity. The kit having one or more compounds of the disclosure could be used in combination with one or more reagent compositions suitable for diagnose a condition as contemplated by this disclosure. For example: a kit could be used for screening and classification of a disease. The expression “in combination with” does not refer to the physical combination or mixing of the reagent compositions, but to their application in separate (consecutive) analysis steps and combination of the data thus obtained.

### EQUIVALENT

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### Example 1: Method of Manufacture

Scheme 1 below provides a possible chemical synthesis route of nonpeptide molecules with the formula 1,3-dioxo-4-methyl-2,3-dihydro-1H-pyrrolo[3,4-c]quinoline that are mainly modified at positions 2 and 8, and the formula 1,4-dioxo-5-methyl-2,3,4-trihydro-1H-pyridazino[4,5-c]quinoline (Formula I) that are mainly modified at positions 2/3 and 9 with a variety of substituents to target and modulate the caspase cascade, wherein, X, R¹, and R² are as specified in the embodiments.

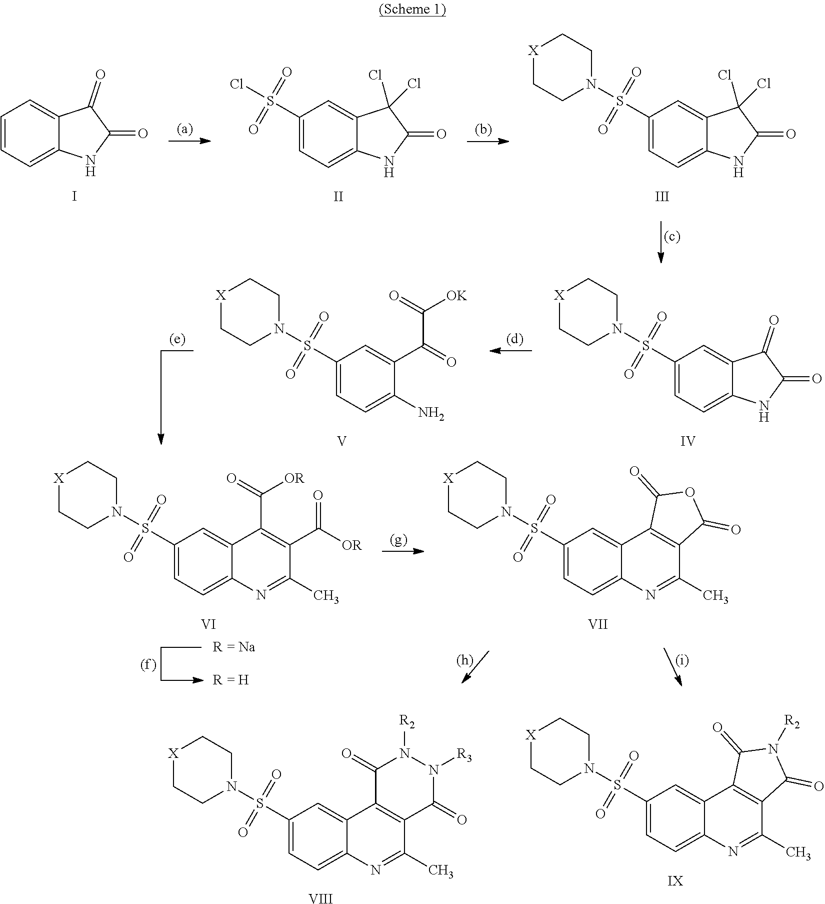

Reagents and conditions: (a) ClSO₃H, 0 to 22° C. (b) morpholine, 0° C. (c) AcOH/H₂O (1:1), reflux (d) KOH (e) methyl acetoacetate, 60° C. (f) HCl, 0° C. (g) Ac₂O, reflux (h,i) phenylhydrazine, pyridine, reflux.

In step (a) of scheme I, solid isotin (compound I) was mixed with concentrated chlorosulfonic acid >99% (13 equiv.) at 0° C. in a round bottom flask and the temperature was raised gradually to 80° C. under reflux for 6 hours. The reaction mixture was cooled down to room temperature and added slowly dropwise to about 200 mL of cold water/ice then left to crystallize at 8° C. for 12 hours. The produced yellow solid (compound 3a) was filtered and washed several times with distilled water then dried under vacuum. The dried product was recrystallized in benzene/acetone, filtered, washed and dried under vacuum (LCMS=300.5 m/z).

In step (b) of scheme I, pure yellow solid, 3,3-dichloro-2-oxo-2,3-dihydro-1H-indole-5-sulfonyl chloride (compound II), was dissolved in THF at 0° C. and stirred for 1 hour. A solution of pyrrolidine, piperidine or morpholine (2 equiv.) in THF was added very slowly drop wise to the reaction mixture at 0° C. and stirred for 2 hour. About 200 mL of cold water/ice was added slowly to the THF solution.

In step (c) of scheme I, a white solid of the conjugated dichloro derivatives was filtered and washed with distilled water then dried under vacuum. The solid was recrystallized in a mixture of hexanes/acetonitrile (5:1) v/v washed with cold acetonitrile and filtered then dried under vacuum. The pure white solid was suspended in water/acetic acid (1:1) v/v solvent and heated under reflux for 12 hours. The solution was cooled down to room temperature and the solvent was evaporated using a rotary evaporator at 80° C. Once the volume got down to one third the original volume, about 200 mL of cold water/ice was added slowly. An orange precipitate (compound IV or derivatives) is formed immediately of the isotin derivative. The orange precipitate was filtered off and washed with distilled water then dried under vacuum.

Compounds Formed from Step (c) could be:

Compound IV or Compound 3c or Compound MO: Chemical name is 5-(morpholine-4-sulfonyl)-2,3-dihydro-1H-indole-2,3-dione. Spectral data: LCMS=297.48 m/z, IR=3482-3317, 1756, 1614, 1348, 1136 and 942 cm-1 1H NMR (400 MHz, DMSO-d6) 11.50 (s, 1H, N—H), 7.91 (dd, J=8.3, 2.0 Hz, 1H, C6-H), 7.69 (d, J=1.9 Hz, 1H, C4-H), 7.13 (d, J=8.3 Hz, 1H, C7-H), 3.63 (t, J=4.7 Hz, 4H, N—CH₂—CH₂—O), 2.87 (t, J=4.7 Hz, 4H, NCH2-CH₂—O).

Compound 3c2 or Compound PY: Chemical name is 5-(pyrrolidine-1-sulfonyl)-2,3-dihydro-1H-indole-2,3-dione. Spectral data: LCMS=281.5 m/z, IR=3417, 1754, 1652, 1618, 1467, 1339 and 1147 cm-1 1H NMR (400 MHz, DMSO-d6) 11.46 (s, 1H, N—H), 7.99 (dd, J=8.3, 1.9 Hz, 1H, C6-H), 7.74 (d, J=1.8 Hz, 1H, C4-H), 7.09 (d, J=8.3 Hz, 1H, C7-H), 3.16-3.11 (m, 4H N—CH₂—CH₂), 1.70-1.65 (m, 4H, N—CH₂—CH₂).

Compound 3c3 or Compound PE: Chemical name is 5-(piperidine-1-sulfonyl)-2,3-dihydro-1H-indole-2,3-dione. Spectral data: LCMS=295.45 m/z, IR=3296, 1745, 1766, 1745, 1617, 1470, and 1334 cm-1 1H NMR (400 MHz, DMSO-d6) 11.47 (d, J=2.8 Hz, 1H, N—H), 7.90 (ddd, J=8.4, 3.3, 1.9 Hz, 1H, C6-H), 7.67 (t, J=2.5 Hz, 1H, C4-H), 7.10 (dd, J=8.3, 3.0 Hz, 1H, C7-H), 2.88 (m, J=4.3, 3.3 Hz, 4H, N—CH₂—CH₂—CH₂—), 1.54 (m, 4H, N—CH₂—CH₂—CH₂—), 1.36 (m, 2H, N—CH₂—CH₂—CH₂—).

In step (d) to (f) of scheme I, a suspension of solid isotin/substituted isotin was prepared using 50 mL of distilled water in a 250 mL round bottom flask and the flask was flushed with a steady flow of N2 gas and stirred for 30 minutes. A volume of 50 mL potassium peroxide (20%) solution (8 equivalents) was added dropwise slowly to the isotin suspension and stirred at room temperature for 1 hour. Methyl acetoacetate solution (3 equivalents) was then added to the reaction mixture dropwise slowly while stirring and left for 12 hrs at 60° C. The reaction mixture was cooled to 0° C. for one hour and the medium was acidified with 20 mL of conc HCl dropwise slowly. The produced white precipitate (compound 1d) was filtered and washed several times with distilled water then dried under vacuum overnight. The white precipitate, 2-methyl-6-[substituted]-3,4-quinoline dicarboxylic acid, was purified by recrystallization in methanol and dried under vacuum. The percentage yield was 65 to 75%.

In step (g) of scheme I, involves the formation of the anhydride of 4-substituted-8-(substituted)-furo[3,4-c]quinoline-1,3dione. The solid crude product of step f was dissolved in 15-20 mL of acetic anhydride and transferred to a 50 mL round bottom flask which was stirred and heated under reflux for 2 hours till the solid was dissolved completely. The reaction mixture was cooled to room temperature and then kept at −20° C. for 4 hours. The solid precipitate was filtered and washed twice with diethyl ether (2×10 mL) and twice with extra pure hexanes (2×10 mL) and then dried under vacuum for 4 hours at 75° C. The produced orange precipitate is 4-methyl-8(substituted)-furo[3,4-c]quinoline-1,3-dione.

In step (h) and (I) of scheme I, involves the formation of 2-(substituted)-4-methyl-8-(substituted)-1,3-dihydropyrrolo[3,4-c]quinoline1,3-diones. The precipitate produced from step (g) was dissolved in 10 mL of pyridine and transferred to a 50 mL round bottom flask. An equivalent amount of the primary amine R²NH² (1 equivalent) was added to the reaction mixture which was heated to 50° C. for 1 h. Acetic anhydride (10 mL) was then added to the reaction mixture and the temperature was raised to 80° C. for 2 hours. The solvent (pyridine/acetic anhydride) was withdrawn from the reaction mixture using a rotary evaporator under vacuum at 70° C. The reaction mixture was allowed to cool down to room temperature and then further cooled a temperature of −20° C. for 3 hours. The residue is then filtered and washed 3 times with (10 mL) Isopropanol. The precipitate was dried under vacuum for 3 hours. The produced solid was 2-(substituted)-4-methyl-8-(substituted)-1,3-dihydropyrrolo[3,4-c]quinoline-1,3-diones.

For compounds with a 6 membered ring pharmacophore with the general formula 1,4-dioxo-5-methyl-2,3,4trihydro-1H-pyridazino[4,5-c]quinoline that are mainly modified at positions 2/3 and 9 with a variety of substituents.

### Example 2-Substitution of Side Chain of Compounds

More than 70 compounds were designed, synthesized and characterized. Each compound was given an ID code that indicates the different substituents at positions 2 and 8. Each ID code consists of a letter or two which indicates the substitution at position 8, for example, compound codes starting with the letter H contain a hydrogen atom at position 8. The letter F indicates a fluorine atom, the letters PY indicate the presence of pyrrolidine-1-sulfonyl group, the letters PE indicate the presence of piperidine-1-sulfonyl group and the letters MO indicates the presence of morpholine-1-sulfonyl group. The other aspect of the compound coding strategy involves the numbers 1, 2, 4, 5, 6A, 6B, 7, 9, 10, 11, 13, 14, 15 and 16 which refer to the many different substituents at position 2 (the imide N) of the molecular scaffold with compounds 6A and 6B being the only exception because they represent a different nucleus of the molecular scaffold.

Table 1 shows a representative list of compounds of the same scaffold (1,4-dioxo-5-methyl-2,3,4-trihydro-1H-pyridazino[4,5-c]quinoline) (Formula I) that are mainly modified at positions 2, 3 and 9).

| R² | R¹ | —H | —F | 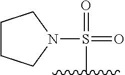 [SMILES: *S(=O)(=O)N1CCCC1] | 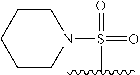 [SMILES: *S(=O)(=O)N1CCCCC1] | 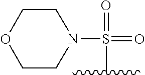 [SMILES: *S(=O)(=O)N1CCOCC1] |
|---|---|---|---|---|---|---|
| 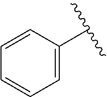 [SMILES: *c1ccccc1] | | H1 | F1 | PY1 | PE1 | MO1 |
| 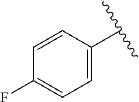 [SMILES: *c1ccc(F)cc1] | | H2 | F2 | PY2 | PE2 | MO2 |
| 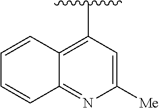 [SMILES: *c1cc(C)nc2ccccc12] | | H4 | F4 | PY4 | PE4 | MO4 |
| 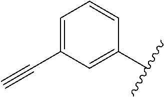 [SMILES: *c1cccc(C#C)c1] | | H5 | F5 | PY5 | PE5 | MO5 |
| 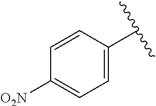 [SMILES: *c1ccc([N+](=O)[O-])cc1] | | H7 | F7 | PY7 | PE7 | MO7 |
| 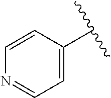 [SMILES: *c1ccncc1] | | H6 | F9 | PY9 | PE9 | MO9 |
| 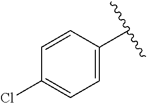 [SMILES: *c1ccc(Cl)cc1] | | H10 | F10 | PY10 | PE10 | M10 |
| 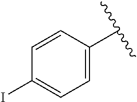 [SMILES: *c1ccc(I)cc1] | | H11 | F11 | PY11 | PE11 | MO11 |
| 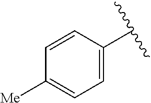 [SMILES: *c1ccc(C)cc1] | | H13 | F13 | PY13 | PE13 | MO13 |
| 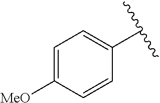 [SMILES: *c1ccc(OC)cc1] | | H14 | F14 | PY14 | PE14 | MO14 |
| 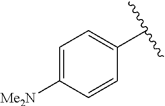 [SMILES: *c1ccc(N(C)C)cc1] | | H15 | F15 | PY15 | PE15 | MO15 |
| 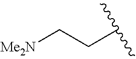 [SMILES: *CCN(C)C] | | H16 | F16 | PY16 | PE16 | MO16 |

Table 2 shows compounds with possible substituents on the molecular scaffold 4-methyl-2-substituted-1H,2H,3Hpyrrolo[3,4-c]quinoline-1,3-dione

| R² | R¹ | —H | —F | 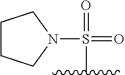 [SMILES: *S(=O)(=O)N1CCCC1] | 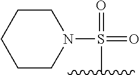 [SMILES: *S(=O)(=O)N1CCCCC1] | 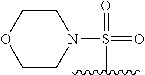 [SMILES: *S(=O)(=O)N1CCOCC1] |
|---|---|---|---|---|---|---|
| —H | | H4 | F4 | PY4 | PE4 | MO4 |

Whereas R¹ is a sulfonamide group with a cyclic amino moiety chosen from pyrrolidine, piperidinyl, or morpholinyl. Also, R² is H, CH₃, allylic, normal alkyl, branched alkyl, unsaturated alkyl, cyclic alkyl, a halogen, —SO₃H, —COOH, —NO₂, —CHO, —CN, —NH₂, —NR₂. Or any derivative or salt thereof. These inhibitors shoed differentially higher inhibitory potency against caspase 7 compared to caspase 3 and caspase 1.

Table 3 shows possible substituents on the molecular scaffold 4-methyl-2-substituted-1H,2H,3Hpyrrolo[3,4-c]quinoline-1,3-dione.

| R² | R³ | R¹ | —H | —F | 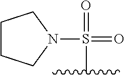 [SMILES: *S(=O)(=O)N1CCCC1] | 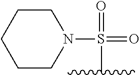 [SMILES: *S(=O)(=O)N1CCCCC1] | 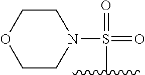 [SMILES: *S(=O)(=O)N1CCOCC1] |
|---|---|---|---|---|---|---|---|
| 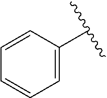 [SMILES: *c1ccccc1] | —H | | H6A | F6A | PY6A | PE6A | MO6A |
| —H | 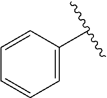 [SMILES: *c1ccccc1] | | H6B | F6B | PY6B | PE6B | MO6B |

whereas R¹ is a sulfonamide group with a cyclic amino moiety chosen from pyrrolidine, piperidinyl, or morpholinyl. Also, R² and R³ can be H, CH₃, allylic, normal alkyl, branched alkyl, unsaturated alkyl, cyclic alkyl, or a phenyl substituted (at any position) with a halogen, —SO₃H, —COOH, —OH, —NO₂, —CHO, —CN, —NH₂, —NR₂, or any derivative or salt thereof. These molecules have demonstrated higher potency inhibiting a broad array of caspases.

In an embodiment, the 6 membered tetrahydropyridazino structures are constitutional isomers. The two compounds have been synthesized in the same experiment and have a slight difference in polarity and can be separated using column chromatography. Also, this difference in polarity was manifested in the detection of about 1 minute difference in lag time of the TIC of the LCMS. However, the 1H NMR spectra are close enough to each other that firm assignments of the structures could not be confirmed. Therefore, quantum mechanics calculations were performed on both structures in order to determine the dipole moment values.

### Example 3: Purification

The purification of the 70 compounds in Tables (1-3) was done utilizing column chromatography and this technique was monitored by TLC plates and a 254 nm wavelength UV light source. The solvent system used for the separation of all these molecules was the (hexanes/ethyl acetate) mixture with a variety of proportions depending on the polarity of each molecule individually. Only the compounds with the ID ending with the number 16 were separated using (dichloro methane/methanol).

### Example 4: Characterization

The successful synthesis of these molecules was confirmed using 1H-NMR and IR spectral analysis. The 1HNMR spectra were collected on Bruker DRX 400 instrument, and in difficult cases compared with spectra predicted by ChemAxon 1H-NMR predictor feature and Mestrenova 10.0 software. Melting points were performed on a Mel-Temp® apparatus. The IR experiments were performed using KBr pellets. The samples were prepared by homogenously mixing about 1 mg of the solid inhibitor to about 100 mg of dry pure KBr. A sufficient amount was compressed to a thin pellet and IR spectra were collected after 32 scans using Nicolet 6700 FT-IR Thermo Scientific® instrument. The purity of the compounds was determined using LCMS and the melting point strategies. LCMS is a powerful technique that has very high sensitivity, making it useful in many applications. Its application is oriented towards the separation, general detection and potential identification of chemicals. The LCMS stock solutions were prepared in extra pure acetonitrile solutions at a concentration of 1 mg/mL. For qualitative purposes, the samples were prepared by mixing 50-100 μL of the stock solution into 1000 to 1200 μL of double deionized water that was previously acidified with formic acid (at a concentration of 0.1% v/v). The liquid chromatography part was done using reversed phase technique utilizing acetonitrile/water solvent system. The mass spectrometry in most cases results in the detection of the M+1 ion which is 1 m/z unit higher than the actual molecular mass of each molecule. The instrument used was Waters® micromass ZQ which uses ESI technique in the mass spectrometer detector. The column used was Atlantis® T3 3 μm. part number is 186003719. Atlantis T3 columns are silica-based, reversed-phase C18 columns that provide balanced retention of polar and hydrophobic molecules. Formic acid was added (0.1% v/v) during sample preparation to reduce the droplet size, increase the conductivity and act as a source of protons. When the organic solvent is heated and evaporated, droplets are formed. The molecules capture a proton to produce the M+1 signal. The purity of these molecules was estimated to be as low as 94% and as high as 98%.

### Spectra Data:

Compound H1: Chemical name is 4-methyl-2-phenyl-1H,2H,3H-pyrrolo[3,4-c]quinoline-1,3-dione LCMS=289.6 m/z, IR=1777, 1716, 1384 and 1116 cm-1 1H NMR (400 MHz, DMSO-d6) 8.72 (dd, J=8.3, 1.4 Hz, 1H, C9-H), 8.15 (d, J=8.5 Hz, 1H, C6-H), 8.00 (ddd, J=8.5, 6.9, 1.5 Hz, 1H, C7-H), 7.85 (ddd, J=8.2, 6.9, 1.2 Hz, 1H, C8-H), 7.58 (dd, J=8.5, 6.9 Hz, 2H, C3-H and C5-H of the phenyl ring at position 2), 7.50 (d, J=7.8 Hz, 3H, C2-H, C4-H and C6-H of the phenyl ring at position 2), 2.96 (s, 3H, C4-CH₃).

Compound H2: Chemical name is 2-(4-fluorophenyl)-4-methyl-1H,2H,3H-pyrrolo[3,4-c]quinoline-1,3-dione LCMS=307.7 m/z, IR=1769, 1720, 1511, 1391, 1234 and 1117 cm-1 1H NMR (400 MHz, DMSO-d6) 8.71 (d, J=8.3 Hz, 1H, C9-H), 8.15 (d, J=8.5 Hz, 1H, C6-H), 8.00 (t, J=7.7 Hz, 1H, C7-H), 7.85 (t, J=7.6 Hz, 1H, C8-H), 7.55 (dd, J=8.5, 5.0 Hz, 2H, C3-H and C5-H of the phenyl ring at position 2), 7.43 (t, J=8.6 Hz, 2H C3-H and C5-H of the phenyl ring at position 2), 2.96 (s, 3H, C4-CH₃).

Compound H4: Chemical name is 4-methyl-2-(2-methylquinolin-4-yl)-1H,2H,3H-pyrrolo[3,4-c]quinoline-1,3-dione LCMS=354.8 m/z, IR=1774, 1719, 1603, 1510, 1414 and 1095 cm-1 1H NMR (400 MHz, DMSO-d6) 8.74 (d, J=8.3 Hz, 1H, C9-H), 8.22 (d, J=8.7 Hz, 1H, C3-H of the quinoline ring at position 2), 8.10-8.02 (m, 3H, overlapping C8-H and C5-H of the quinoline ring at position 2 with C6H), 7.85 (dddd, J=23.9, 8.5, 6.9, 1.4 Hz, 2H, overlapping C7-H of the quinoline ring at position 2 with C7-H), 7.63 (d, J=1.4 Hz, 1H, C6-H of the quinoline ring at position 2), 7.60-7.54 (m, 1H, C8-H), 3.00 (d, J=1.3 Hz, 3H, C4-CH₃), 2.76 (s, 3H, C2-CH₃ of the quinoline ring at position 2).

Compound H5: Chemical name is 2-(3-ethynylphenyl)-4-methyl-1H,2H,3H-pyrrolo[3,4-c]quinoline-1,3-dione LCMS=313.7 m/z, IR=3295, 2346, 1764, 1717, 1383 and 1116 cm-1 1H NMR (400 MHz, DMSO-d6) 8.73 (d, J=8.3 Hz, 1H, C9-H), 8.17 (d, J=8.5 Hz, 1H, C6-H), 8.02 (ddd, J=7.9, 6.7, 1.2 Hz, 1H, C7-H), 7.87 (t, J=7.6 Hz, 1H, C8-H), 7.64-7.55 (m, 4H, overlapping C2-H, C4-H, C5-H and C6-H of the phenyl ring at position 2), 4.35 (s, 1H, C3-C C—H of the phenyl ring at position 2), 2.98 (s, 3H, C5—CH3).

Compound H6A: Chemical name is 5-methyl-3-phenyl-1H,2H,3H,4H-pyridazino[4,5-c]quinoline-1,4-dione LCMS=304.65 m/z, IR=3271, 1775, 1655, 1618, 1599, 1496, 1265, 1235 and 997 cm-1 1H NMR (400 MHz, DMSO-d6) 11.08 (s, 1H, N—H), 8.79 (d, J=8.3 Hz, 1H, C10-H), 8.12 (d, J=8.5 Hz, 1H, C7-H), 7.99 (t, J=7.6 Hz, 1H, C9-H), 7.87 (t, J=7.5 Hz, 1H, C8-H), 7.47 (d, J=8.1 Hz, 2H, C2-H and C6-H of the phenyl ring at position 3), 7.37 (t, J=7.7 Hz, 2H, C3-H and C5-H of the phenyl ring at position 3), 6.97 (t, J=7.1 Hz, 1H, C4-H of the phenyl ring at position 3), 2.89 (s, 3H, C5-CH₃).

Compound H6B: Chemical name is 5-methyl-2-phenyl-1H,2H,3H,4H-pyridazino[4,5-c]quinoline-1,4-dione LCMS=304.65 m/z, IR=3299, 1761, 1720, 1633, 1599, 1511, 1494, 1448, 1377, 1268, 1240, 1136, 1104 and 952 cm-1 1H NMR (400 MHz, DMSO-d6) 10.92 (s, 1H, N—H), 8.57 (d, J=8.1 Hz, 1H, C10-H), 8.15 (d, J=8.6 Hz, 1H, C7-H), 7.86 (dt, J=27.4, 7.0 Hz, 2H, overlapping C8-H and C9-H), 7.42-7.25 (m, 4H, overlapping C2-H, C3-H, C5-H and C6-H of the phenyl ring at position 2), 6.90 (d, J=7.5 Hz, 1H, C4-H of the phenyl ring at position 2), 3.02 (s, 3H, C4—CH3).

Compound H7: Chemical name is 4-methyl-2-(4-nitrophenyl)-1H,2H,3H-pyrrolo[3,4-c]quinoline-1,3-dione LCMS=334.68 m/z, IR=1769, 1722, 1625, 1608, 1525, 1383 and 1350 cm-1 1H NMR (400 MHz, DMSO-d6) 8.78-8.74 (m, 1H, C9-H), 8.48-8.43 (d, 2H, C3-H and C5-H of the phenyl ring at position 2), 8.19 (d, J=8.6 Hz, 1H, C6-H), 8.04 (ddd, J=8.6, 6.9, 1.5 Hz, 1H, C7-H), 7.92-7.81 (m, 3H, overlapping C2-H and C6-H of the phenyl ring at position 2 with C8-H), 3.00 (s, 3H, C4—CH3).

Compound H9: Chemical name is 4-methyl-2-(pyridin-4-yl)-1H,2H,3H-pyrrolo[3,4-c]quinoline-1,3-dione LCMS=290.5 m/z, IR=1775, 1721, 1623, 1589, 1503, 1382, 1112 and 1100 cm-1 1H NMR (400 MHz, DMSO-d6) 8.76 (dd, J=18.8, 6.8 Hz, 3H, overlapping C9-H with the two protons ortho to N of the pyridine ring at position 2), 8.17 (d, J=8.5 Hz, 1H, C6-H), 8.02 (ddd, J=8.6, 6.9, 1.5 Hz, 1H, C7-H), 7.87 (t, J=7.7 Hz, 1H), 7.66-7.59 (m, 2H, the two protons meta to N of the pyridine ring at position 2), 2.98 (s, 3H, C4—CH3).

Compound H10: Chemical name is 2-(4-chlorophenyl)-4-methyl-1H,2H,3H-pyrrolo[3,4-c]quinoline-1,3-dione LCMS=323.44 m/z, IR=1769, 1721, 1625, 1495, 1384, 1117 and 1089 cm-1 1H NMR (400 MHz, DMSO-d6) 8.74 (d, J=8.4 Hz, 1H, C9-H), 8.17 (d, J=8.6 Hz, 1H, C6-H), 8.07-7.99 (m, 1H, C7-H), 7.86 (td, J=7.6, 6.9, 1.3 Hz, 1H, C8-H), 7.68-7.63 (d, 2H, C3-H and C5-H of the phenyl ring at position 2), 7.54 (d, J=8.8, 2.4 Hz, 2H, C2-H and C6-H of the phenyl ring at position 2), 2.97 (s, 3H, C4—CH3).

Compound H11: Chemical name is 2-(4-iodophenyl)-4-methyl-1H,2H,3H-pyrrolo[3,4-c]quinoline-1,3-dione LCMS=415.8 m/z, IR=1771, 1719, 1489, 1386, 1373 and 1099 cm-1 1H NMR (400 MHz, DMSO-d6) 8.75-8.69 (m, 1H, C9-H), 8.16 (d, J=8.4 Hz, 1H, C6-H), 8.01 (ddd, J=8.5, 6.9, 1.5 Hz, 1H, C7-H), 7.96-7.93 (d, 2H, C2-H and C6-H of the phenyl ring at position 2), 7.85 (ddd, J=8.2, 6.9, 1.2 Hz, 1H, C8-H), 7.34-7.30 (d, 2H, C3-H and C5-H of the phenyl ring at position 2), 2.96 (s, 3H, C4—CH3).

Compound H13: Chemical name is 4-methyl-2-(4-methylphenyl)-1H,2H,3H-pyrrolo[3,4-c]quinoline-1,3-dione LCMS=303.78 m/z, IR=1771, 1719, 1652, 1516, 1385, 1373 and 1130 cm-1 1H NMR (400 MHz, DMSO-d6) 8.74 (d, J=8.0 Hz, 1H, C9-H), 8.17 (d, J=8.5 Hz, 1H, C6-H), 8.01 (ddd, J=8.6, 6.8, 1.5 Hz, 1H, C7-H), 7.85 (t, J=7.9 Hz, 1H, C8-H), 7.37 (s, 4H, overlapping C2-H, C3-H, C5-H and C6-H of the phenyl ring at position 2), 2.97 (s, 3H, C4—CH3), 2.40 (s, 3H, C4—CH3 of the phenyl ring at position 2).

Compound H14: Chemical name is 2-(4-methoxyphenyl)-4-methyl-1H,2H,3H-pyrrolo[3,4-c]quinoline-1,3-dione LCMS=319.7 m/z, IR=1767, 1717, 1516, 1396, 1248, 1190 and 1121 cm-1 1H NMR (400 MHz, DMSO-d6) 8.73 (d, J=8.2 Hz, 1H), 8.16 (d, J=8.5 Hz, 1H), 8.00 (s, 1H), 7.85 (t, J=7.5 Hz, 1H), 7.40 (d, J=8.3 Hz, 2H, C3-H and C5-H of the phenyl ring at position 2), 7.11 (d, J=8.3 Hz, 2H, C2-H and C6-H of the phenyl ring at position 2), 3.83 (s, 3H, C4-OCH3 of the phenyl ring at position 2), 2.97 (s, 3H, C4—CH3).

Compound H15: Chemical name is 2-[4-(N,N-dimethylamino)phenyl]-4-methyl-1H,2H,3H-pyrrolo[3,4-c]quinoline-1,3-dione LCMS=332.66 m/z, IR=2822, 1772, 1714, 1610, 1523, 1447, 1395 and 1169 cm-1 1H NMR (400 MHz, Chloroform-d) 8.87 (d, J=8.0 Hz, 1H, C9-H), 8.15 (d, J=8.5 Hz, 1H, C6-H), 7.93-7.86 (m, 1H, C7-H), 7.72 (dd, J=9.5, 5.6 Hz, 1H, C8-H), 7.25 (d, J=8.6 Hz, 2H, C2-H and C6-H of the phenyl ring at position 2), 6.83 (d, J=8.6 Hz, 2H, C3-H and C5-H of the phenyl ring at position 2), 3.09 (d, J=4.2 Hz, 3H, C4—CH3), 3.02 (d, J=4.1 Hz, 6H, N—CH3).

Compound H16: Chemical name is 2-[2-(dimethylamino)ethyl]-4-methyl-1H,2H,3H-pyrrolo[3,4-c]quinoline-1,3-dione LCMS=284.7 m/z, IR=1766, 1703, 1397, 1351, 1109 and 933 cm-1 1H NMR (400 MHz, Chloroform-d) 8.81 (d, J=8.4 Hz, 1H, C9-H), 8.12 (d, J=8.6 Hz, 1H, C6-H), 7.87 (ddd, J=8.6, 6.8, 1.5 Hz, 1H, C7-H), 7.74-7.66 (m, 1H, C8-H), 3.86 (t, J=6.5 Hz, 2H, N—CH2—CH2—N(CH3)2), 3.05 (s, 3H, C4—CH3), 2.64 (d, J=6.4 Hz, 2H, N—CH2—CH2-N(CH3)2), 2.32 (s, 6H, N—CH3).

Compound F1: Chemical name is 2-phenyl-4-methyl-8-fluoro-1,3-dihydropyrrolo[3,4-c]quinoline-1,3-dione LCMS=307 m/z, IR=1774, 1724, 1500, 1401, 1387, 1198, 1114 and 1096 cm-1 1H NMR (400 MHz, DMSO-d6) 8.34 (dd, J=8.8, 2.9 Hz, 1H, C9-H), 8.27 (dd, J=9.5, 5.2 Hz, 1H, C6-H), 8.00-7.92 (m, 1H, C7-H), 7.61-7.54 (m, 2H, C3-H and C5-H of the phenyl ring at position 2), 7.52-7.46 (m, 3H, overlapping C2-H, C4-H and C6-H of the phenyl ring at position 2), 2.97 (s, 3H, C4—CH3).

Compound F2: Chemical name is 8-fluoro-2-(4-fluorophenyl)-4-methyl-1H,2H,3H-pyrrolo[3,4-c]quinoline-1,3-dione LCMS=325.5 m/z, IR=1769, 1722, 1602, 1513, 1389, 1230, 1196, 1160 and 1112 cm-1 1H NMR (400 MHz, DMSO-d6) 8.36-8.31 (m, 1H, C9-H), 8.27 (dd, J=9.4, 5.3 Hz, 1H, C6-H), 7.96 (t, J=7.6 Hz, 1H, C7-H), 7.54 (dd, J=8.6, 5.2 Hz, 2H, C3-H and C5-H of the phenyl ring at position 2), 7.43 (t, J=8.8 Hz, 2H, C2-H and C6-H of the phenyl ring at position 2), 2.97 (s, 3H, C4—CH3).

Compound F4: Chemical name is 8-fluoro-4-methyl-2-(2-methylquinolin-4-yl)-1H,2H,3H-pyrrolo[3,4-c]quinoline-1,3-dione LCMS=372.57 m/z, IR=1781, 1719, 1605, 1514, 1468, 1401, 1316, 1293, 1224, 1186 and 1103 cm-1 1H NMR (400 MHz, DMSO-d6) 8.31 (d, J=9.3 Hz, 2H, overlapping of C9-H with C3-H of the quinoline ring at position 2), 8.08 (d, J=8.5 Hz, 2H, overlapping C8-H and C5-H of the quinoline ring at position 2), 8.01 (d, J=9.8 Hz, 1H, C6-H), 7.82 (t, J=7.7 Hz, 1H, C7-H), 7.61 (s, 1H, C7-H of the quinoline ring at position 2), 7.60-7.54 (m, 1H, C6-H of the quinoline ring at position 2), 2.99 (s, 3H, C4—CH3), 2.75 (d, J=1.1 Hz, 3H, C2—CH3 of the quinoline ring at position 2).

Compound F5: Chemical name is 2-(3-ethynylphenyl)-8-fluoro-4-methyl-1H,2H,3H-pyrrolo[3,4-c]quinoline-1,3-dione LCMS=331.54 m/z, IR=3266, 2346, 1772, 1723, 1604, 1512, 1490, 1436, 1406, 1386, 1280, 1182, 1125, 1112 and 1101 cm-1 1H NMR (400 MHz, DMSO-d6) 8.34 (dd, J=8.9, 3.0 Hz, 1H, C9-H), 8.28 (dd, J=9.4, 5.4 Hz, 1H, C6-H), 7.97 (td, J=8.9, 3.0 Hz, 1H, C7-H), 7.64-7.52 (m, 4H, overlapping C2-H, C4-H, C5-H and C6-H of the phenyl ring at position 2), 4.35 (s, 1H, C3-C C—H of the phenyl ring at position 2)), 2.97 (s, 3H, C4—CH3).

Compound F6A: Chemical name is 9-fluoro-5-methyl-3-phenyl-1H,2H,3H,4H-pyridazino[4,5-c]quinoline-1,4-dione LCMS=322.54 m/z, IR=3282, 1775, 1764, 1619, 1596, 1517, 1496, 1446, 1262, 1237, 1221, 1170, 1129, 1002 and 958 cm-1 1H NMR (400 MHz, DMSO-d6) 11.12 (s, 1H, N—H), 8.35 (s, 1H, C10-H), 8.21 (s, 1H, C7-H), 7.93 (d, J=8.7 Hz, 1H, C8-H), 7.51-7.35 (m, 4H, overlapping C2-H, C3-H, C5-H and C6-H of the phenyl ring at position 3), 6.98 (d, J=8.0 Hz, 1H, C4-H of the phenyl ring at position 3), 2.87 (s, 3H, C5—CH3).

Compound F6B: Chemical name is 9-fluoro-5-methyl-2-phenyl-1H,2H,3H,4H-pyridazino[4,5-c]quinoline-1,4-dione LCMS=322.54 m/z, IR=3287, 1777, 1750, 1618, 1598, 1560, 1510, 1495, 1449, 1376, 1263, 1238, 1222, 1179, 1130 and 957 cm-1 1H NMR (400 MHz, DMSO-d6) 11.00 (s, 1H, N—H), 8.22 (dt, J=9.2, 4.6 Hz, 1H, C10-H), 8.15 (dd, J=8.7, 3.9 Hz, 1H, C7-H), 7.80 (t, J=9.0 Hz, 1H, C8-H), 7.40-7.30 (m, 4H, overlapping C2-H, C3-H, C5-H and C6-H of the phenyl ring at position 2), 6.92 (s, 1H, C4-H of the phenyl ring at position 2), 3.00 (d, J=3.9 Hz, 3H, C5CH3).

Compound F7: Chemical name is 8-fluoro-4-methyl-2-(4-nitrophenyl)-1H,2H,3H-pyrrolo[3,4-c]quinoline-1,3-dione LCMS=352.54 m/z, IR=1772, 1725, 1610, 1523, 1473, 1405, 1383, 1349, 1265, 1199, 1130, 1118 and 1086 cm-1 1H NMR (400 MHz, DMSO-d6) 8.46 (d, J=9.0 Hz, 2H, C3-H and C5-H of the phenyl ring at position 2), 8.35 (dd, J=8.8, 2.9 Hz, 1H, C9-H), 8.29 (dd, J=9.3, 5.4 Hz, 1H, C6-H), 7.99 (td, J=8.8, 2.9 Hz, 1H, C7-H), 7.83 (d, J=9.0 Hz, 2H, C2-H and C56H of the phenyl ring at position 2), 2.99 (s, 3H, C4—CH3).

Compound F9: Chemical name is 8-fluoro-4-methyl-2-(pyridin-4-yl)-1H,2H,3H-pyrrolo[3,4-c]quinoline-1,3-dione LCMS=308.58 m/z, IR=1769, 1727, 1611, 1587, 1504, 1472, 1403, 1382, 1217, 1199, 1134, 1098 and 1078 cm-1 1H NMR (400 MHz, DMSO-d6) 8.81-8.77 (d, 2H, the two protons ortho to N of the pyridine ring at position 2), 8.35 (dd, J=8.9, 2.9 Hz, 1H, C9-H), 8.29 (dd, J=9.4, 5.4 Hz, 1H, C6-H), 7.98 (td, J=8.9, 3.0 Hz, 1H, C7H), 7.65-7.59 (d, 2H, the two protons meta to N of the pyridine ring at position 2), 2.99 (s, 3H, C4—CH3).

Compound F10: Chemical name is 2-(4-chlorophenyl)-8-fluoro-4-methyl-1H,2H,3H-pyrrolo[3,4-c]quinoline-1,3-dione LCMS=341.5 m/z, IR=1768, 1724, 1613, 1513, 1494, 1470, 1452, 1407, 1385, 1179 and 1092 cm-1 1H NMR (400 MHz, DMSO-d6) 8.33 (dd, J=8.8, 3.0 Hz, 1H, C9-H), 8.27 (dd, J=9.3, 5.2 Hz, 1H, C6-H), 7.97 (td, J=8.9, 3.0 Hz, 1H, C7-H), 7.69-7.65 (d, 2H, C3-H and C5-H of the phenyl ring at position 2), 7.56-7.52 (d, 2H, C2-H and C6-H of the phenyl ring at position 2), 2.97 (s, 3H, C4—CH3).

Compound F11: Chemical name is 8-fluoro-2-(4-iodophenyl)-4-methyl-1H,2H,3H-pyrrolo[3,4-c]quinoline-1,3-dione LCMS=433.5 m/z, IR=1766, 1709, 1609, 1512, 1490, 1467, 1393, 1370, 1265, 1239, 1197, 1179, 1115, 1098 and 1004 cm-1 1H NMR (400 MHz, DMSO-d6) 8.33 (dd, J=8.9, 2.9 Hz, 1H, C9-H), 8.27 (dd, J=9.4, 5.3 Hz, 1H, C6-H), 7.99-7.93 (m, 3H, overlapping C7-H with C2-H and C6H of the phenyl ring at position 2), 7.35-7.28 (d, 2H, C3-H and C5H of the phenyl ring at position 2), 2.96 (s, 3H, C4—CH3).

Compound F13: Chemical name is 8-fluoro-4-methyl-2-(4-methylphenyl)-1H,2H,3H-pyrrolo[3,4-c]quinoline-1,3-dione LCMS=321.56 m/z, IR=1774, 1723, 1617, 1560, 1516, 1470, 1403, 1384, 1375, 1265, 1233, 1197, 1180, 1123 and 1092 cm-1 1H NMR (400 MHz, DMSO-d6) 8.33 (dd, J=9.0, 3.0 Hz, 1H, C9-H), 8.26 (dd, J=9.4, 5.4 Hz, 1H, C6-H), 7.96 (td, J=8.9, 2.9 Hz, 1H, C7-H), 7.36 (s, 4H, C2-H, C3-H, C5-H and C6-H of the phenyl ring at position 2), 2.96 (s, 3H, C4—CH3), 2.40 (s, 3H, C4—CH3 of the phenyl ring at position 2).

Compound F14: Chemical name is 8-fluoro-2-(4-methoxyphenyl)-4-methyl-1H,2H,3H-pyrrolo[3,4-c]quinoline-1,3-dione LCMS=337.54 m/z, IR=1765, 1717, 1610, 1515, 1470, 1403, 1302, 1252, 1201, 1135, 1118, 1059 and 1035 cm-1 1H NMR (400 MHz, DMSO-d6) 8.30 (dt, J=9.1, 2.1 Hz, 1H, C9-H), 8.27-8.22 (m, 1H, C6-H), 7.98-7.91 (m, 1H, C7-H), 7.42-7.37 (d, 2H, C2-H and C6-H of the phenyl ring at position 2), 7.13-7.09 (d, 2H, C3-H and C5-H of the phenyl ring at position 2), 3.83 (d, J=1.3 Hz, 3H, C4-OCH3 of the phenyl ring at position 2), 2.95 (d, J=1.4 Hz, 3H, C4—CH3).

Compound F15: Chemical name is 2-[4-(N,N-dimethylamino)phenyl]-8-fluoro-4-methyl-1H,2H,3H-pyrrolo[3,4-c]quinoline-1,3dione LCMS=350.8 m/z, IR=2920, 1773, 1610, 1526, 1515, 1450, 1389, 1358, 1339, 1263, 1235, 1205, 1181, 1130 and 1086 cm-1 1H NMR (400 MHz, Chloroform-d) 8.47 (dd, J=8.8, 2.9 Hz, 1H, C9-H), 8.15 (dd, J=9.3, 5.1 Hz, 1H, C6-H), 7.65 (ddd, J=9.3, 8.1, 2.9 Hz, 1H, C7-H), 7.29-7.23 (d, 2H, C2-H and C6-H of the phenyl ring at position 2), 6.88-6.78 (d, 2H, C3-H and C5-H of the phenyl ring at position 2), 3.07 (s, 3H, C4—CH3), 3.02 (s, 6H, N—CH3).

Compound F16: Chemical name is 2-[2-(N,N-dimethylamino)ethyl]-8-fluoro-4-methyl-1H,2H,3H-pyrrolo[3,4-c]quinoline-1,3dione LCMS=302.7 m/z, IR=1768, 1703, 1615, 1563, 1516, 1451, 1394, 1374, 1340, 1232, 1183, 1160, 1107, 1049, 1017, 1002 and 946 cm-1 1H NMR (400 MHz, Chloroform-d) 8.41 (dd, J=8.7, 2.9 Hz, 1H, C9-H), 8.12 (dd, J=9.4, 5.2 Hz, 1H, C6-H), 7.63 (ddd, J=9.4, 8.1, 2.9 Hz, 1H, C7-H), 3.87 (t, J=6.4 Hz, 2H, N—CH2—CH2—N(CH3)2), 3.03 (s, 3H, C4—CH3), 2.66 (t, 2H, N—CH2—CH2—N(CH3)2), 2.32 (s, 6H, N—CH3).

Compound PY1: Chemical name is 4-methyl-2-phenyl-8-(pyrrolidine-1-sulfonyl)-1H,2H,3H-pyrrolo[3,4-c]quinoline-1,3-dione LCMS=422.93 m/z, IR=1772, 1712, 1626, 1595, 1503, 1395, 1336, 1234, 1194, 1158, 1140, 1124, 1107 and 1074 cm-1 1H NMR (400 MHz, DMSO-d6) 9.13 (d, J=2.1 Hz, 1H, C9-H), 8.37 (d, J=8.9 Hz, 1H, C6-H), 8.30 (dd, J=8.9, 2.1 Hz, 1H, C7-H), 7.61-7.56 (m, 2H, C3-H and C5-H of the phenyl ring at position 2), 7.53-7.48 (m, 3H, C2-H, C4-H and C6-H of the phenyl ring at position 2), 3.28-3.24 (m, 4H, N—CH2—CH2-), 3.03 (s, 3H, C4—CH3), 1.68 (t, J=6.6 Hz, 4H, N—CH2—CH2—).

Compound PY2: Chemical name is 2-(4-fluorophenyl)-4-methyl-8-(pyrrolidine-1-sulfonyl)-1H,2H,3H-pyrrolo[3,4-c]quinoline1,3-dione LCMS=441.13 m/z, IR=1772, 1715, 1627, 1601, 1514, 1395, 1337, 1237, 1161, 1140, 1123, 1079 and 1015 cm-1 1H NMR (400 MHz, DMSO-d6) 9.13-9.11 (m, 1H, C9-H), 8.37 (d, J=9.0 Hz, 1H, C6-H), 8.31 (dd, J=9.0, 2.1 Hz, 1H, C7-H), 7.55 (dd, J=9.0, 5.1 Hz, 2H, C3-H and C5-H of the phenyl ring at position 2), 7.44 (t, J=8.8 Hz, 2H, C2-H and C6-H of the phenyl ring at position 2), 3.27-3.24 (m, 4H, N—CH2—CH2-), 3.03 (s, 3H, C4—CH3), 1.70-1.66 (m, 4H, N—CH2—CH2—).

Compound PY4: Chemical name is 4-methyl-2-(2-methylquinolin-4-yl)-8-(pyrrolidine-1-sulfonyl)-1H,2H,3H-pyrrolo[3,4c]quinoline-1,3-dione LCMS=487.88 m/z, IR=1778, 1720, 1627, 1607, 1560, 1509, 1414, 1344, 1315, 1293, 1218, 1149, 1125, 1111, 1089, 1058 and 1004 cm-1 1H NMR (400 MHz, DMSO-d6) 9.11 (d, J=2.0 Hz, 1H, C9-H), 8.41 (d, J=9.0 Hz, 1H, C6-H), 8.34 (dd, J=9.1, 2.1 Hz, 1H, C7-H), 8.09 (dd, J=8.3, 5.0 Hz, 2H, overlapping C8-H and C5-H of the quinoline ring at position 2), 7.83 (t, J=7.6 Hz, 1H, C3-H of the quinoline ring at position 2), 7.60 (d, J=12.4 Hz, 2H, overlapping C6-H and C7-H of the quinoline ring at position 2), 3.28-3.24 (m, 4H, N—CH2—CH2—), 3.05 (s, 3H, C4—CH3), 2.76 (s, 3H, C2—CH3 of the quinoline ring at position 2), 1.71-1.66 (m, 4H, N—CH2—CH2).

Compound PY5: Chemical name is 2-(3-ethynylphenyl)-4-methyl-8-(pyrrolidine-1-sulfonyl)-1H,2H,3H-pyrrolo[3,4-c]quinoline1,3-dione 20 LCMS=446.92 m/z, IR=3264, 2350, 1779, 1726, 1620, 1599, 1579, 1482, 1431, 1394, 1380, 1334, 1238, 1201, 1160, 1126, 1104, 1078 and 1015 cm-1 1H NMR (400 MHz, DMSO-d6) 9.12 (d, J=1.9 Hz, 1H, C9-H), 8.38 (d, J=9.0 Hz, 1H, C6-H), 8.31 (dd, J=9.1, 2.0 Hz, 1H, C7-H), 7.64-7.60 (m, 3H, C4-H, C5-H and C6-H of the phenyl ring at position 2), 7.57 (d, J=5.4 Hz, 1H, C2-H of the phenyl ring at position 2), 4.36 (s, 1H, C3-C C—H of the phenyl ring at position 2), 3.25 (t, J=6.7 Hz, 4H, N—CH2—CH2—), 3.03 (s, J=1.4 Hz, 3H, C4—CH3), 1.71-1.66 (m, 4H, N—CH2—CH2—).

Compound PY6A: Chemical name is 5-methyl-3-phenyl-9-(pyrrolidine-1-sulfonyl)-1H,2H,3H,4H-pyridazino[4,5-c]quinoline-1,4dione LCMS=436.69 m/z, IR=3190, 3089-3059, 1850, 1770, 1601, 1493, 1452, 1374, 1181 and 1081 cm-1 1H NMR (400 MHz, DMSO-d6) 11.33 (s, 1H, N—H), 9.27 (s, 1H, C10-H), 8.29 (m, 2H, overlapping C7-H and C8-H), 7.44 (d, J=7.9 Hz, 2H, C3-H and C5-H of the phenyl ring at position 3), 7.37 (t, J=7.9 Hz, 2H, C2-H and C6-H of the phenyl ring at position 3), 6.99 (t, J=7.3 Hz, 1H, C4-H of the phenyl ring at position 3), 2.93 (s, 3H, C5—CH3), 3.4 (m, 4H, N—CH2—CH2—), 1.68 (t, 4H, N—CH2—CH2—).

Compound PY6B: Chemical name is 5-methyl-2-phenyl-9-(pyrrolidine-1-sulfonyl)-1H,2H,3H,4H-pyridazino[4,5-c]quinoline-1,4dione LCMS=437.69 m/z, IR=3292-3267, 1777, 1735, 1632, 1596, 1535, 1497, 1448, 1349, 1316, 1279, 1265, 1240, 1138, 1069 and 1010 cm-1 1H NMR (400 MHz, DMSO-d6) 11.10 (s, 1H, N—H), 8.94 (d, J=2.1 Hz, 1H, C10-H), 8.35 (d, J=8.9 Hz, 1H, C7-H), 8.20 (dd, J=8.9, 2.1 Hz, 1H, C8-H), 7.40 (d, J=7.9 Hz, 2H, C3-H and C5-H of the phenyl ring at position 2), 7.34 (t, J=7.7 Hz, 2H, C2-H and C6-H of the phenyl ring at position 2), 6.94 (t, J=7.2 Hz, 1H, C4-H of the phenyl ring at position 2), 3.25 (m, 4H, N—CH2—CH2—), 3.07 (s, 3H, C5—CH3), 1.70-1.66 (m, 4H, N—CH2—CH2—).

Compound PY7: Chemical name is 4-methyl-2-(4-nitrophenyl)-8-(pyrrolidine-1-sulfonyl)-1H,2H,3H-pyrrolo[3,4-c]quinoline-1,3dione LCMS=365.9 m/z, IR=3277-3218, 1731, 1681, 1619, 1597, 1503, 1404, 1346, 1332, 1302, 1267, 1112 and 1005 cm-1 1H NMR (400 MHz, DMSO-d6) 9.12 (d, J=2.0 Hz, 1H, C9-H), 8.38 (d, J=9.0 Hz, 1H, C6-H), 8.31 (dd, J=8.9, 2.1 Hz, 1H, C7-H), 8.44 (d, J=8.7 Hz, 2H, C3-H and C5-H of the phenyl ring at position 2), 7.84 (d, J=8.7 Hz, 2H, C2-H and C6-H of the phenyl ring at position 2), 3.26 (m, 4H, N—CH2—CH2—), 3.03 (s, 3H, C4—CH3), 1.71-1.66 (m, 4H, N—CH2—CH2—).

Compound PY9: Chemical name is 4-methyl-2-(pyridin-4-yl)-8-(pyrrolidine-1-sulfonyl)-1H,2H,3H-pyrrolo[3,4-c]quinoline-1,3dione LCMS=423.66 m/z, IR=1779, 1729, 1619, 1585, 1567, 1499, 1419, 1388, 1368, 1337, 1163, 1143, 1075 and 1016 cm-1 1H NMR (400 MHz, DMSO-d6) 9.13 (d, J=2.0 Hz, 1H, C9-H), 8.82-8.78 (m, 2H, the two protons ortho to N of the pyridine ring at position 2), 8.39 (d, J=9.0 Hz, 1H, C6-H), 8.33 (dd, J=8.9, 2.1 Hz, 1H, C7-H), 7.65-7.61 (d, 2H, the two protons meta to N of the pyridine ring at position 2), 3.28-3.24 (m, 4H, N—CH2—CH2—), 3.04 (s, 3H, C4—CH3), 1.71-1.66 (m, 4H, N—CH2—CH2—).

Compound PY10: Chemical name is 2-(4-chlorophenyl)-4-methyl-8-(pyrrolidine-1-sulfonyl)-1H,2H,3H-pyrrolo[3,4-c]quinoline1,3-dione LCMS=456.26 m/z, IR=1775, 1710, 1625, 1595, 1499, 1396, 1342, 1162, 1141, 1127, 1086 and 1017 cm-1 21 1H NMR (400 MHz, DMSO-d6) 9.12 (d, J=1.8 Hz, 1H, C9-H), 8.37 (d, J=9.1 Hz, 1H, C6-H), 8.33-8.29 (m, 1H, C7-H), 7.68 (d, J=8.6, 1.6 Hz, 2H, C3-H and C5-H of the phenyl ring at position 2), 7.57-7.52 (d, 2H, C2H and C6-H of the phenyl ring at position 2), 3.27 (m, J=7.5 Hz, 4H, N—CH2—CH2—), 3.03 (s, J=1.4 Hz, 3H, C4CH3), 1.68 (m, J=7.2 Hz, 4H, N—CH2—CH2—).

Compound PY11: Chemical name is 2-(4-iodophenyl)-4-methyl-8-(pyrrolidine-1-sulfonyl)-1H,2H,3H-pyrrolo[3,4-c]quinoline-1,3dione LCMS=548.25 m/z, IR=1774, 1709, 1622, 1594, 1492, 1395, 1337, 1268, 1160, 1140, 1127, 1110, 1092 and 1010 cm-1 1H NMR (400 MHz, DMSO-d6) 9.11 (s, 1H, C9-H), 8.37 (d, J=8.9 Hz, 1H), 8.31 (d, J=7.7 Hz, 1H), 7.96 (d, J=8.4 Hz, 2H, C2-H and C6-H of the phenyl ring at position 2), 7.32 (d, J=8.6 Hz, 2H, C3-H and C5-H of the phenyl ring at position 2), 3.26 (m, 4H, N—CH2—CH2—), 3.02 (s, 3H, C4—CH3), 1.68 (m, 4H, N—CH2—CH2—).

Compound PY13: Chemical name is 4-methyl-2-(4-methylphenyl)-8-(pyrrolidine-1-sulfonyl)-1H,2H,3H-pyrrolo[3,4-c]quinoline1,3-dione LCMS=436.64 m/z, IR=1777, 1722, 1623, 1513, 1394, 1345, 1163, 1142, 1127 and 1075 cm-1 1H NMR (400 MHz, DMSO-d6) 9.12 (d, J=2.1 Hz, 1H, C9-H), 8.36 (d, J=8.9 Hz, 1H, C6-H), 8.30 (dd, J=9.0, 2.1 Hz, 1H, C7-H), 7.38 (s, 4H, overlapping C2-H, C3-H, C5-H and C6-H of the phenyl ring at position 2), 3.27-3.23 (m, 4H, N—CH2—CH2—), 3.02 (s, 3H, C4—CH3), 2.40 (s, 3H, C4—CH3 of the phenyl ring at position 2), 1.71-1.65 (m, 4H, N—CH2—CH2—)

Compound PY14: Chemical name c]quinoline-1,3-dione is 2-(4-methoxyphenyl)-4-methyl-8-(pyrrolidine-1-sulfonyl)-1H,2H,3H-pyrrolo[3,4LCMS=452.62 m/z, IR=1779, 1720, 1629, 1508, 1396, 1338, 1300, 1244, 1199, 1176, 1159, 1141 and 1101 cm-1 1H NMR (400 MHz, DMSO-d6) 9.12 (d, J=2.1 Hz, 1H, C9-H), 8.36 (d, J=9.0 Hz, 1H, C6-H), 8.31-8.27 (m, 1H, C7-H), 7.43-7.37 (d, 2H, C3-H and C5-H of the phenyl ring at position 2), 7.12 (d, J=8.7 Hz, 2H, C2-H and C6-H of the phenyl ring at position 2), 3.84 (s, 3H, C4-OCH3 of the phenyl ring at position 2), 3.25 (m, 4H, N—CH2—CH2—), 3.02 (s, 3H, C4—CH3), 1.69 (d, J=7.1 Hz, 4H, N—CH2—CH2—).

Compound PY15: Chemical name is 2-[4-(N,N-dimethylamino)phenyl]-4-methyl-8-(pyrrolidine-1-sulfonyl)-1H,2H,3Hpyrrolo[3,4-c]quinoline-1,3-dione LCMS=465.75 m/z, IR=1776, 1715, 1630, 1607, 1521, 1400, 1339, 1261, 1158, 1141, 1107, 1072 and 1003 cm-1 1H NMR (400 MHz, DMSO-d6) 9.12 (d, J=2.2 Hz, 1H, C9-H), 8.35 (d, J=9.0 Hz, 1H, C6-H), 8.28 (dd, J=9.0, 2.1 Hz, 1H, C7-H), 7.25 (d, J=9.1, 2.2 Hz, 2H, C2-H and C6-H of the phenyl ring at position 2), 6.87-6.82 (d, 2H, C3-H and C5-H of the phenyl ring at position 2), 3.25 (t, J=3.4 Hz, 4H, N—CH2—CH2—), 3.01 (s, 3H, C4CH3), 2.97 (s, 6H, N—CH3), 1.68 (m, J=2.4 Hz, 4H, N—CH2—CH2—).

Compound PY16: Chemical name is 2-[2-(N,N-dimethylamino)ethyl]-4-methyl-8-(pyrrolidine-1-sulfonyl)-1H,2H,3H-pyrrolo[3,4c]quinoline-1,3-dione LCMS=417.66 m/z, IR=2922, 1768, 1714, 1623, 1600, 1492, 1451, 1397, 1353, 1264, 1173, 1110 and 1072 cm-1 1H NMR (400 MHz, Chloroform-d) 9.27 (dd, J=1.8, 0.9 Hz, 1H, C9-H), 8.23 (dt, J=3.1, 1.6 Hz, 2H, overlapping C6-H and C7-H), 3.87 (t, J=6.3 Hz, 2H, N—CH2—CH2—N(CH3), 3.39-3.35 (t, 4H, N—CH2—CH2—), 3.09 (s, 3H, C4—CH3), 2.65 (s, 2H, N—CH2—CH2—N(CH3), 2.31 (s, 6H, N—CH3), 1.85-1.77 (m, 4H, N—CH2—CH2—).

Compound PE1: Chemical name is 4-methyl-2-phenyl-8-(piperidine-1-sulfonyl)-1H,2H,3H-pyrrolo[3,4-c]quinoline-1,3-dione LCMS=436.7 m/z, IR=1774, 1714, 1627, 1595, 1502, 1394, 1339, 1312, 1277, 1164, 1142, 1125, 1105, 1075 and 1053 cm-1 1H NMR (400 MHz, DMSO-d6) 9.08 (d, J=2.0 Hz, 1H, C9-H), 8.38 (dd, J=9.0, 1.6 Hz, 1H, C6-H), 8.21 (dt, J=8.8, 1.9 Hz, 1H, C7-H), 7.62-7.56 (d, 2H, C3-H and C5-H of the phenyl ring at position 2), 7.53-7.47 (m, 3H, overlapping C2-H, C4-H and C6-H of the phenyl ring at position 2), 3.03 (m, J=1.6 Hz, 7H, C4—CH3 overlapping with N—CH2—CH2—CH2), 1.56 (m, 4H, N—CH2—CH2—CH2), 1.36 (m, 2H, N—CH2—CH2—CH2).

Compound PE2: Chemical name is 2-(4-fluorophenyl)-4-methyl-8-(piperidine-1-sulfonyl)-1H,2H,3H-pyrrolo[3,4-c]quinoline1,3-dione LCMS=454.64 m/z, IR=1778, 1724, 1623, 1597, 1512, 1394, 1379, 1342, 1225, 1169, 1142, 1127, 1100, 1089, 1072, 1000 and 930 cm-1 1H NMR (400 MHz, DMSO-d6) 9.07 (d, J=2.1 Hz, 1H, C9-H), 8.38 (d, J=9.0 Hz, 1H, C6-H), 8.22 (dd, J=9.1, 2.1 Hz, 1H, C7-H), 7.58-7.53 (m, 2H, C3-H and C5-H of the phenyl ring at position 2), 7.44 (m, J=8.8 Hz, 2H, C2-H and C6-H of the phenyl ring at position 2), 3.02 (m, J=4.6 Hz, 7H, C4—CH3 overlapping with N—CH2CH2—CH2), 1.56 (m, 4H, N—CH2—CH2—CH2), 1.36 (m, 2H, N—CH2—CH2—CH2).

Compound PE4: Chemical name is 4-methyl-2-(2-methylquinolin-4-yl)-8-(piperidine-1-sulfonyl)-1H,2H,3H-pyrrolo[3,4c]quinoline-1,3-dione LCMS=501.4 m/z, IR=1776, 1721, 1625, 1509, 1411, 1400, 1363, 1342, 1315, 1159 and 1148 cm-1 1H NMR (400 MHz, DMSO-d6) 9.07 (s, 1H, C9-H), 8.42 (d, J=9.0 Hz, 1H, C6-H,), 8.28-8.22 (m, 1H, C7H), 8.14-8.04 (m, 2H, overlapping C8-H and C5-H of the quinoline ring at position 2), 7.83 (t, J=7.7 Hz, 1H, C3-H of the quinoline ring at position 2), 7.60 (d, J=12.5 Hz, 2H, overlapping C6-H and C7-H of the quinoline ring at position 2), 3.04 (m, J=8.8 Hz, 7H, C4—CH3 overlapping with N—CH2—CH2—CH2), 2.76 (s, 3H, C2—CH3 of the quinoline ring at position 2), 1.56 (m, 4H, N—CH2—CH2—CH2), 1.37 (m, 2H, N—CH2—CH2—CH2).

Compound PE5: Chemical name is 2-(3-ethynylphenyl)-4-methyl-8-(piperidine-1-sulfonyl)-1H,2H,3H-pyrrolo[3,4-c]quinoline1,3-dione LCMS=460.72 m/z, IR=3253, 2345, 1775, 1720, 1624, 1599, 1577, 1500, 1480, 1431, 1364, 1335, 1282, 1209, 1164, 1152, 1125, 1105, 1077, 1051 and 935 cm-1 1H NMR (400 MHz, DMSO-d6) 9.08 (d, J=1.7 Hz, 1H, C9-H), 8.41-8.36 (m, 1H, C6-H), 8.25-8.20 (m, 1H, C7-H), 7.64-7.55 (m, 4H, overlapping C2-H, C4-H, C5-H and C6-H of the phenyl ring at position 2), 4.36 (s, 1H, C3-C C—H of the phenyl ring at position 2), 3.03 (m, J=1.5 Hz, 7H, C4—CH3 overlapping with N—CH2—CH2—CH2), 1.56 (m, 4H, N—CH2—CH2—CH2), 1.36 (m, 2H, N—CH2—CH2—CH2).

Compound PE6A: Chemical name is 5-methyl-3-phenyl-9-(piperidine-1-sulfonyl)-1H,2H,3H,4H-pyridazino[4,5-c]quinoline-1,4dione LCMS=451.7 m/z, IR=3256, 3081-3059, 1786, 1631, 1601, 1582, 1493, 1452, 1338, 1257, 1230, 1164, 1144, 1074, 1027 and 965 cm-1 1H NMR (400 MHz, DMSO-d6) 11.34 (s, 1H, N—H), 9.20 (d, J=2.1 Hz, 1H, C10-H), 8.30 (d, J=8.9 Hz, 1H, C7-H), 8.20 (dd, J=9.0, 2.1 Hz, 1H, C8-H), 7.43 (d, J=7.9 Hz, 2H, C3-H and C5-H of the phenyl ring at position 3), 7.37 (t, J=7.8 Hz, 2H, C2-H and C6-H of the phenyl ring at position 3), 6.99 (t, J=7.2 Hz, 1H, C4-H of the phenyl ring at position 3), 3.05 (m, J=5.3 Hz, 4H, N—CH2—CH2—CH2), 2.93 (s, 3H, C5—CH3), 1.57 (m, 4H, NCH2—CH2—CH2), 1.35 (m, 2H, N—CH2—CH2—CH2).

Compound PE6B: Chemical name is 5-methyl-2-phenyl-9-(piperidine-1-sulfonyl)-1H,2H,3H,4H-pyridazino[4,5-c]quinoline-1,4dione LCMS=451.7 m/z, IR=3270, 3082-3059, 1777, 1600, 1492, 1451, 1375, 1181, 1070, 1028 and 965 cm-1 1H NMR (400 MHz, DMSO-d6) 11.34 (s, 1H, N—H), 9.20 (d, J=2.0 Hz, 1H, C10-H), 8.30 (d, J=8.9 Hz, 1H, C7-H), 8.23-8.18 (m, 1H, C8-H), 7.43 (d, J=8.0 Hz, 2H, C3-H and C5-H of the phenyl ring at position 2), 7.37 (t, J=7.8 Hz, 2H, C2-H and C6-H of the phenyl ring at position 2), 6.99 (t, J=7.2 Hz, 1H, C4-H of the phenyl ring at position 2), 3.04 (m, J=5.8 Hz, 4H, N—CH2—CH2—CH2), 2.93 (m, 3H, C5—CH3), 1.57 (s, 4H, N—CH2—CH2—CH2), 1.35 (m, 2H, N—CH2—CH2—CH2).

Compound PE7: Chemical name is 4-methyl-2-(4-nitrophenyl)-8-(piperidine-1-sulfonyl)-1H,2H,3H-pyrrolo[3,4-c]quinoline-1,3dione LCMS=481.65 m/z, IR=1780, 1732, 1622, 1596, 1520, 1499, 1473, 1392, 1372, 1343, 1166, 1140, 1126, 1081, 1070 and 933 cm-1 1H NMR (400 MHz, DMSO-d6) 9.09 (s, 1H, C9-H), 8.47 (d, J=8.8 Hz, 2H, C3-H and C5-H of the phenyl ring at position 2), 8.40 (d, J=8.9 Hz, 1H, C6-H), 8.24 (d, J=8.6 Hz, 1H, C7-H), 7.84 (d, J=8.9 Hz, 2H, C2-H and C6-H of the phenyl ring at position 2), 3.06-3.00 (m, 7H, C4—CH3 overlapping with N—CH2—CH2—CH2), 1.56 (m, 4H, N—CH2—CH2—CH2), 1.37 (m, 2H, N—CH2—CH2—CH2).

Compound PE9: Chemical name is 4-methyl-8-(piperidine-1-sulfonyl)-2-(pyridin-4-yl)-1H,2H,3H-pyrrolo[3,4-c]quinoline-1,3dione LCMS=437.84 m/z, IR=1781, 1729, 1622, 1585, 1566, 1498, 1471, 1417, 1390, 1370, 1341, 1319, 1281, 1267, 1167, 1144, 1126, 1103, 1071, 1052 and 931 cm-1 1H NMR (400 MHz, DMSO-d6) 9.08 (d, J=2.2 Hz, 1H, C9-H), 8.83-8.77 (m, 2H, the two protons ortho to N of the pyridine ring at position 2), 8.39 (d, J=9.0 Hz, 1H, C6-H), 8.24 (dd, J=8.9, 2.2 Hz, 1H, C7-H), 7.66-7.60 (m, 2H, the two protons meta to N of the pyridine ring at position 2), 3.03 (m, J=10.7 Hz, 7H, C4—CH3 overlapping with N—CH2—CH2—CH2), 1.56 (m, 4H, N—CH2—CH2—CH2), 1.36 (m, 2H, N—CH2—CH2—CH2).

Compound PE10: Chemical name is 2-(4-chlorophenyl)-4-methyl-8-(piperidine-1-sulfonyl)-1H,2H,3H-pyrrolo[3,4-c]quinoline1,3-dione LCMS=470.3 m/z, IR=1778, 1725, 1621, 1594, 1495, 1392, 1376, 1342, 1168, 1142, 1126, 1090, 1072, 1052, 1015 and 930 cm-1 1H NMR (400 MHz, DMSO-d6) 9.07 (d, J=2.0 Hz, 1H, C9-H), 8.38 (d, J=8.9 Hz, 1H, C6-H), 8.22 (dt, J=8.9, 1.4 Hz, 1H, C7-H), 7.71-7.64 (d, 2H, C3-H and C5-H of the phenyl ring at position 2), 7.58-7.51 (d, 2H, C2-H and C6-H of the phenyl ring at position 2), 3.05-2.99 (m, 7H, C4—CH3 overlapping with N—CH2—CH2—CH2), 1.56 (m, 4H, N—CH2—CH2—CH2), 1.36 (m, 2H, N—CH2—CH2—CH2).

Compound PE11: Chemical name is 2-(4-iodophenyl)-4-methyl-8-(piperidine-1-sulfonyl)-1H,2H,3H-pyrrolo[3,4-c]quinoline-1,3dione LCMS=562.6 m/z, IR=1775, 1717, 1624, 1492, 1396, 1340, 1280, 1163, 1140, 1125, 1107, 1074, 1053, 1009 and 932 cm-1 1H NMR (400 MHz, DMSO-d6) 9.06 (s, 1H, C9-H), 8.38 (d, J=9.0 Hz, 1H, C6-H), 8.22 (dd, J=9.0 Hz, 1H, C7-H), 7.96 (d, J=8.3 Hz, 2H, C2-H and C6-H of the phenyl ring at position 2), 7.32 (d, J=8.1 Hz, 2H, C3-H and C5-H of the phenyl ring at position 2), 3.04-2.99 (m, 7H, C4—CH3 overlapping with N—CH2—CH2—CH2), 1.56 (m, 4H, N—CH2—CH2—CH2), 1.36 (m, 2H, N—CH2—CH2—CH2).

Compound PE13: Chemical name is 4-methyl-2-(4-methylphenyl)-8-(piperidine-1-sulfonyl)-1H,2H,3H-pyrrolo[3,4-c]quinoline1,3-dione LCMS=451.2 m/z, IR=1775, 1420, 1625, 1516, 1397, 1339, 1169, 1143, 1073 and 943 cm-1 1H NMR (400 MHz, DMSO-d6) 9.07 (d, J=2.0 Hz, 1H, C9-H), 8.37 (d, J=8.9 Hz, 1H, C6-H), 8.21 (dd, J=9.0, 2.1 Hz, 1H, C7-H), 7.37 (s, 4H, C2-H, C3-H, C5-H and C6-H of the phenyl ring at position 2), 3.04-2.99 (m, 7H, C4—CH3 overlapping with N—CH2—CH2—CH2), 2.40 (s, 3H, C4—CH3 of the phenyl ring at position 2), 1.56 (m, 4H, N—CH2—CH2—CH2), 1.36 (m, 2H, N—CH2—CH2—CH2).

Compound PE14: Chemical name is 2-(4-methoxyphenyl)-4-methyl-8-(piperidine-1-sulfonyl)-1H,2H,3H-pyrrolo[3,4-c]quinoline1,3-dione LCMS=466.72 m/z, IR=1774, 1715, 1627, 1514, 1395, 1342, 1315, 1249, 1166, 1142, 1125, 1097, 1035 and 933 cm-1 1H NMR (400 MHz, DMSO-d6) 9.07 (d, J=2.1 Hz, 1H, C9-H), 8.37 (d, J=9.0 Hz, 1H, C6-H), 8.21 (dd, J=9.0, 2.1 Hz, 1H, C7-H), 7.40 (d, J=8.7 Hz, 2H, C3-H and C5-H of the phenyl ring at position 2), 7.12 (d, J=8.9 Hz, 2H, C2-H and C6-H of the phenyl ring at position 2)), 3.84 (s, 3H, C4-OCH3 of the phenyl ring at position 2), 3.02 (m, 7H, C4—CH3 overlapping with N—CH2—CH2—CH2), 1.56 (m, 4H, N—CH2—CH2—CH2), 1.37 (m, 2H, NCH2—CH2—CH2).

Compound PE15: Chemical name is 2-[4-(N,N-dimethylamino)phenyl]-4-methyl-8-(piperidine-1-sulfonyl)-1H,2H,3H-pyrrolo[3,4c]quinoline-1,3-dione LCMS=479.7 m/z, IR=2923, 1774, 1716, 1629, 1601, 1521, 1493, 1451, 1400, 1354, 1335, 1169, 1141, 1104, 1073, 1027 and 940 cm-1 1H NMR (400 MHz, DMSO-d6) 9.07 (d, J=2.1 Hz, 1H, C9-H), 8.36 (d, J=9.0 Hz, 1H, C6-H), 8.20 (dd, J=9.1, 2.3 Hz, 1H, C7-H), 7.25 (d, J=8.9 Hz, 2H, C2-H and C6-H of the phenyl ring at position 2), 6.87-6.81 (m, 2H, C3-H and C5-H of the phenyl ring at position 2), 3.01 (m, J=3.6 Hz, 7H, C4—CH3 overlapping with N—CH2CH2—CH2), 2.97 (s, 6H, N—CH3), 1.56 (m, 4H, N—CH2—CH2—CH2), 1.36 (m, 2H, N—CH2—CH2—CH2).

Compound PE16: Chemical name is 2-[2-(N,N-dimethylamino)ethyl]-4-methyl-8-(piperidine-1-sulfonyl)-1H,2H,3H-pyrrolo[3,4c]quinoline-1,3-dione LCMS=432.13 m/z, IR=2926, 1770, 1711, 1623, 1594, 1493, 1468, 1398, 1358, 1343, 1321, 1174,1111, 1052 and 937 cm-1 1H NMR (400 MHz, Chloroform-d) 9.20 (d, 1H, C9-H), 8.24 (d, 1H, C6-H), 8.15 (dd, 1H, C7-H), 3.88 (t, 2H, N—CH2—CH2—N(CH3)2), 3.09 (m, 7H, C4—CH3 overlapping with N—CH2—CH2—CH2), 2.66 (t, 2H, N—CH2—CH2N(CH3)2), 2.31 (s, 6H, N—CH3), 1.67 (m, 4H, N—CH2—CH2—CH2), 1.45 (m, 2H, N—CH2—CH2—CH2).

Compound MO1: Chemical name is 4-methyl-8-(morpholine-4-sulfonyl)-2-phenyl-1H,2H,3H-pyrrolo[3,4-c]quinoline-1,3-dione LCMS=438.66 m/z, IR=1777, 1723, 1620, 1595, 1499, 1455, 1391, 1371, 1349, 1302, 1263, 1229, 1167, 1142, 1110, 10841 and 952 cm-1 1H NMR (400 MHz, DMSO-d6) 9.10 (d, J=2.1 Hz, 1H, C9-H), 8.40 (d, J=9.0 Hz, 1H, C6-H), 8.22 (dd, J=9.0, 2.1 Hz, 1H, C7-H), 7.62-7.56 (m, 2H, C3-H and C5-H of the phenyl ring at position 2), 7.53-7.48 (m, 3H, overlapping C2-H, C4-H and C6-H of the phenyl ring at position 2), 3.65 (t, J=4.6 Hz, 4H, O—CH2—CH2—N), 3.04 (s, 3H, C4—CH3), 3.01 (t, J=4.7 Hz, 4H, O—CH2—CH2—N).

Compound MO2: Chemical name is 2-(4-fluorophenyl)-4-methyl-8-(morpholine-4-sulfonyl)-1H,2H,3H-pyrrolo[3,4-c]quinoline1,3-dione LCMS=456.6 m/z, IR=1778, 1723, 1621, 1598, 1512, 1454, 1393, 1350, 1464, 1300, 12227, 1169, 1143, 1126, 1110, 1080 and 950 cm-1 1H NMR (400 MHz, DMSO-d6) 9.09 (d, J=2.0 Hz, 1H, C9-H), 8.41 (d, J=9.0 Hz, 1H, C6-H), 8.22 (dd, J=8.9, 2.1 Hz, 1H, C7-H), 7.55 (dd, J=8.8, 5.2 Hz, 2H, C3-H and C5-H of the phenyl ring at position 2), 7.44 (m, J=8.8 Hz, 2H, C2-H and C6-H of the phenyl ring at position 2), 3.65 (t, J=4.8 Hz, 4H, O—CH2—CH2—N), 3.04 (s, 3H, C4—CH3), 3.00 (t, J=4.8 Hz, 4H, O—CH2—CH2-N).

Compound MO5: Chemical name is 2-(3-ethynylphenyl)-4-methyl-8-(morpholine-4-sulfonyl)-1H,2H,3H-pyrrolo[3,4-c]quinoline1,3-dione LCMS=462.67 m/z, IR=3284, 2352, 1772, 1720, 1624, 1598, 1577, 1500, 1482, 1454, 1429, 1395, 1351, 1299, 1292, 1167, 1130, 1111, 1079 and 949 cm-1 1H NMR (400 MHz, DMSO-d6) 9.09 (d, J=2.1 Hz, 1H, C9-H), 8.41 (d, J=9.0 Hz, 1H, C6-H), 8.23 (dd, J=9.0, 2.1 Hz, 1H, C7-H), 7.63-7.54 (m, 4H, overlapping C2-H, C4-H, C5-H and C6-H of the phenyl ring at position 2), 4.36 (s, 1H, C3-C C—H of the phenyl ring at position 2), 3.65 (t, J=4.7 Hz, 4H, O—CH2—CH2—N), 3.04 (s, 3H, C4—CH3), 3.01 (t, J=5.0 Hz, 4H, O—CH2—CH2—N).

Compound MO4: Chemical name is 4-methyl-2-(2-methylquinolin-4-yl)-8-(morpholine-4-sulfonyl)-1H,2H,3H-pyrrolo[3,4c]quinoline-1,3-dione LCMS=503.86 m/z, IR=1777, 1718, 1625, 1560, 1508, 1457, 1417, 1365, 1348, 1316, 1299, 1262, 1233, 1159, 1151, 1110, 1090, 1072 and 947 cm-1 1H NMR (400 MHz, DMSO-d6) 9.09 (d, J=2.0 Hz, 1H, C9-H), 8.45 (d, J=9.0 Hz, 1H, C6-H), 8.28-8.23 (m, 1H, C7-H), 8.09 (dd, J=8.3, 4.1 Hz, 2H, overlapping C8-H and C5-H of the quinoline ring at position 2), 7.83 (t, J=7.7 Hz, 1H, C3-H of the quinoline ring at position 2), 7.63-7.56 (m, 2H, overlapping C6-H and C7-H of the quinoline ring at position 2), 3.65 (t, J=5.0 Hz, 4H, O—CH2—CH2—N), 3.06 (s, 3H, C4—CH3), 3.01 (t, 4H, O—CH2CH2—N), 2.76 (s, 3H, C2—CH3 of the quinoline ring at position 2).

Compound MO6A: Chemical name is 5-methyl-9-(morpholine-4-sulfonyl)-3-phenyl-1H,2H,3H,4H-pyridazino[4,5-c]quinoline-1,4dione LCMS=453.6 m/z, IR=3266, 3082-3059, 1774, 1600, 1582, 1692, 1452, 1352, 1261, 1181, 1153, 1112, 1028, 1004 and 965 cm-1 1H NMR (400 MHz, DMSO-d6) 11.35 (s, 1H, N—H), 9.22 (d, J=2.1 Hz, 1H, C10-H), 8.33 (d, J=8.9 Hz, 1H, C7-H), 8.20 (dd, J=9.0, 2.1 Hz, 1H, C8-H), 7.43 (d, J=8.0 Hz, 2H, C3-H and C5-H of the phenyl ring at position 3), 7.37 (t, J=7.9 Hz, 2H, C2-H and C6-H of the phenyl ring at position 3), 6.99 (t, J=7.3 Hz, 1H, C4-H of the phenyl ring at position 3), 3.66 (t, J=4.9 Hz, 4H, O—CH2—CH2—N), 3.03 (t, J=4.7 Hz, 4H, O—CH2—CH2—N), 2.94 (s, 3H, C5—CH3).

Compound MO6B: Chemical name is 5-methyl-9-(morpholine-4-sulfonyl)-2-phenyl-1H,2H,3H,4H-pyridazino[4,5-c]quinoline-1,4dione LCMS=453.6 m/z, IR=3276, 1778, 1726, 1618, 1597, 1530, 1497, 1449, 1344, 1264, 1235, 1169, 1158, 1140, 1114, 1081 and 952 cm-1 1H NMR (400 MHz, DMSO-d6) 11.12 (s, 1H, N—H), 8.89 (d, J=2.1 Hz, 1H, C10-H), 8.38 (d, J=8.7 Hz, 1H, C7-H), 8.12 (dd, J=8.8, 2.2 Hz, 1H, C8-H), 7.43-7.32 (m, 4H, overlapping C2-H, C3-H, C5-H and C6-H of the phenyl ring at position 2), 6.95 (t, J=7.2 Hz, 1H, C4-H of the phenyl ring at position 2), 3.66 (t, J=4.7 Hz, 4H, O—CH2—CH2—N), 3.08 (s, 3H, C5—CH3), 3.00 (t, 4H, O—CH2—CH2—N).

Compound MO7: Chemical name is 4-methyl-8-(morpholine-4-sulfonyl)-2-(4-nitrophenyl)-1H,2H,3H-pyrrolo[3,4-c]quinoline1,3-dione LCMS=483.6 m/z, IR=1782, 1728, 1621, 1608, 1595, 1523, 1499, 1454, 1393, 1373, 1344, 1301, 1264, 1230, 1166, 1141, 1126, 1112, 1080 and 950 cm-1 1H NMR (400 MHz, DMSO-d6) 9.10 (d, J=2.0 Hz, 1H, C9-H), 8.49-8.45 (d, 2H, C3-H and C5-H of the phenyl ring at position 2), 8.42 (d, J=8.9 Hz, 1H, C6-H), 8.24 (dd, J=9.0, 2.1 Hz, 1H, C7-H), 7.87-7.81 (d, 2H, C2-H and C6-H of the phenyl ring at position 2), 3.66 (t, J=4.6 Hz, 4H, O—CH2—CH2—N), 3.05 (s, 3H, C4—CH3), 3.02 (d, J=5.0 Hz, 4H, O—CH2—CH2—N).

Compound MO9: Chemical name is 4-methyl-8-(morpholine-4-sulfonyl)-2-(pyridin-4-yl)-1H,2H,3H-pyrrolo[3,4-c]quinoline-1,3dione LCMS=439.64 m/z, IR=1781, 1729, 1621, 1585, 1567, 1504, 1453, 1417, 1390, 1369, 1351, 1301, 1263, 1229, 1168, 1144, 1131, 1122, 1079, 1004, 992 and 951 cm-1 1H NMR (400 MHz, DMSO-d6) 9.10 (d, J=2.3 Hz, 1H, C9-H), 8.80 (d, J=5.2 Hz, 2H, the two protons ortho to N of the pyridine ring at position 2), 8.42 (d, J=8.8 Hz, 1H, C6-H), 8.27-8.21 (m, 1H, C7-H), 7.65-7.60 (d, 2H, the two protons meta o to N of the pyridine ring at position 2), 3.66 (t, 4H, O—CH2—CH2—N), 3.05 (d, J=1.6 Hz, 3H, C4—CH3), 3.01 (t, 4H, O—CH2—CH2—N).

Compound MO10: Chemical name is 2-(4-chlorophenyl)-4-methyl-8-(morpholine-4-sulfonyl)-1H, 2H,3H-pyrrolo[3,4-c]quinoline1,3-dione LCMS=472.57 m/z, IR=1780, 1724, 1622, 1595, 1496, 1496, 1466, 1455, 1392, 1350, 1333, 1301, 1265, 1167, 1143, 1125, 1112, 1091, 1080, 1015, 1000 and 951 cm-1 1H NMR (400 MHz, DMSO-d6) 9.09 (d, J=2.1 Hz, 1H, C9-H), 8.41 (d, J=9.0 Hz, 1H, C6-H), 8.22 (dd, J=9.0, 2.1 Hz, 1H, C7-H), 7.70-7.65 (d, 2H, C3-H and C5-H of the phenyl ring at position 2), 7.57-7.52 (d, 2H, C2-H and C6-H of the phenyl ring at position 2), 3.65 (t, J=4.6 Hz, 4H, O—CH2—CH2—N), 3.04 (s, 3H, C4—CH3), 3.01 (t, J=4.8 Hz, 4H, O—CH2—CH2—N).

Compound MO11: Chemical name is 2-(4-iodophenyl)-4-methyl-8-(morpholine-4-sulfonyl)-1H,2H,3H-pyrrolo[3,4-c]quinoline1,3-dione LCMS=564.55 m/z, IR=1775, 1716, 1626, 1597, 1492, 1453, 1394, 1349, 1261, 1144, 1164, 1124, 1113, 1081, 1003 and 950 cm-1 1H NMR (400 MHz, DMSO-d6) 9.08 (s, 1H, C9-H), 8.41 (d, J=9.0 Hz, 1H, C6-H), 8.22 (d, J=9.0 Hz, 1H, C7H), 7.96 (d, J=8.4 Hz, 2H, C2-H and C6-H of the phenyl ring at position 2), 7.32 (d, J=8.2 Hz, 2H, C3-H and C5-H of the phenyl ring at position 2), 3.65 (t, 4H, O—CH2—CH2—N), 3.03 (s, 3H, C4—CH3), 3.00 (t, 4H, O—CH2CH2—N).

Compound MO13: Chemical name is 4-methyl-2-(4-methylphenyl)-8-(morpholine-4-sulfonyl)-1H,2H,3H-pyrrolo[3,4-c]quinoline1,3-dione LCMS=452.7 m/z, IR=1776, 1721, 1624, 1598, 1514, 1458, 1395, 1352, 1305, 1263, 1230, 1168, 1144, 1128, 1110, 1081 and 949 cm-1 1H NMR (400 MHz, DMSO-d6) 9.09 (d, J=2.1 Hz, 1H, C9-H), 8.40 (d, J=8.9 Hz, 1H, C6-H), 8.21 (dd, J=9.0, 2.1 Hz, 1H, C7-H), 7.37 (s, 4H, overlapping C2-H, C3-H, C5-H and C6-H of the phenyl ring at position 2), 3.65 (t, J=4.6 Hz, 4H, O—CH2—CH2—N), 3.03 (s, 3H, C4—CH3), 3.01 (t, J=5.0 Hz, 4H, O—CH2—CH2—N), 2.40 (s, 3H, C4—CH3 of the phenyl group at position 2).

Compound MO14: is 2-(4-methoxyphenyl)-4-methyl-8-(morpholine-4-sulfonyl)-1H,2H,3H-pyrrolo[3,4Chemical name c]quinoline-1,3-dione LCMS=468.67 m/z, IR=1776, 1720, 1624, 1514, 1456, 1397, 1350, 1300, 1250, 1169, 1144, 1127, 1110, 1081, 1024, 999 and 950 cm-1 1H NMR (400 MHz, DMSO-d6) 9.09 (s, 1H, C9-H), 8.40 (d, J=9.0 Hz, 1H, C6-H), 8.21 (d, J=8.2 Hz, 1H, C7H), 7.40 (d, J=8.9 Hz, 2H, C3-H and C4-H of the phenyl ring at position 2), 7.12 (d, J=8.9 Hz, 2H, C2-H and C6-H of the phenyl ring at position 2), 3.84 (s, 3H, C4-OCH3 of the phenyl ring at position 2), 3.65 (t, 4H, OCH2—CH2—N), 3.05-2.96 (m, 7H, overlapping C4—CH3 with, O—CH2—CH2—N).

Compound MO15: Chemical name is 2-[4-(N,N-dimethylamino)phenyl]-4-methyl-8-(morpholine-4-sulfonyl)-1H,2H,3Hpyrrolo[3,4-c]quinoline-1,3-dione LCMS=481.72 m/z, IR=1772, 1713, 1626, 1611, 1525, 1451, 1400, 1358, 1297, 1261, 1235, 1205, 1168, 1147, 1129, 1111, 1102, 1080 and 951 cm-1 1H NMR (400 MHz, Chloroform-d) 9.30-9.27 (m, 1H, C9-H), 8.30 (d, J=9.0 Hz, 1H, C6-H), 8.17 (dd, J=9.0, 1.9 Hz, 1H, C7-H), 7.25 (d, J=8.1 Hz, 2H, C2-H and C6-H of the phenyl ring at position 2) 6.83 (d, J=8.1 Hz, 2H, C3-H and C4-H of the phenyl ring at position 2), 3.78-3.75 (t, 4H, O—CH2—CH2—N), 3.13 (m, J=9.8, 2.7 Hz, 7H, overlapping C4—CH3 with, O—CH2—CH2—N), 3.03 (s, 6H, N—CH3).

Compound MO16: Chemical name is 2-[2-(N,N-dimethylamino)ethyl]-4-methyl-8-(morpholine-4-sulfonyl)-1H,2H,3H-pyrrolo[3,4c]quinoline-1,3-dione LCMS=434.16 m/z, IR=1769, 1713, 1623, 1594, 1560, 1501, 1451, 1397, 1353, 1263, 1220, 1172, 1110, 1073, 1040 and cm-1 1H NMR (400 MHz, Chloroform-d) 9.23 (dd, J=4.6, 2.1 Hz, 1H, C9-H), 8.27 (dd, J=9.0, 4.4 Hz, 1H, C6-H), 8.14 (ddd, J=9.0, 4.5, 2.2 Hz, 1H, C7-H), 3.89 (t, 2H, N—CH2—CH2—N(CH3)2), 3.78 (t, J=4.8, 4.3 Hz, 4H, OCH2—CH2—N), 3.16-3.10 (m, 7H, overlapping C4—CH3 with O—CH2—CH2—N), 2.68 (t, 2H, N—CH2—CH2—N(CH3)2), 2.32 (s, J=4.2 Hz, 6H, N—CH3).

### Example 5: In Vitro Caspase Inhibitory Assay

Caspase 3 screening assays were performed similar to the assays described by Okun et al. [61]. The caspase 3 inhibitor drug screening kits were purchases from Raybiotech Company (Caspase 3: Item number is 68SR-Casp3S100), (Caspase 7: Item number is 68SR-Casp7-S100), and (Caspase 1: Item number is 68FL-Casp1-S200). The direct fluorometric enzyme assay was performed in a 96 well plate which was read in a temperature-controlled plate reader equipped with a 400-nm excitation filter and 505-nm emission filter. The fluorescence was read every one minute for 60 minutes.

Caspase 3 screening and IC50 assay procedure: 1) The tested compounds were dissolved in DMSO to a concentration of 2.5 mM. A volume of 1 μL of DMSO solution was added to 24 μL H₂O to produce a final volume of 25 μL/well in a 96 well plate. A volume of 2.5 μL of the Active caspase 3 was added. 2) A positive inhibition control was prepared by adding 1 μL of the caspase 3 Inhibitor (Z-DEVD-FMK, provided in the kit) instead of the tested compounds. 3) The other positive inhibition control was prepared by adding 1 μL of the caspase 3/7 Inhibitor (Compound C2, purchased from ApexBio company A1925) instead of the tested compounds. 4) A negative inhibition control was prepared using pure DMSO without the inhibitor. A volume of 1 μL of pure DMSO to 24 μL H₂O to a final volume of 25 μL/well. 5) Master Mix was prepared for each assay containing the follows: a. 22.5 μL 2× Reaction Buffer (containing 10 mM DTT). b. 2.5 μL of the 1 mM Ac-DEVD-AFC substrate (50 μM final concentration). 6) Master Mix was mixed well then 22.5 μL of the Master Mix was added quickly to each well to start the reaction. 7) Samples were read in Gemini EM fluorescence Microplate reader with a 400 nm excitation wavelength and 505 nm emission wavelength every 1 minute for duration of 1 hour at 37° C. 8) Data was exported in an Excel spread sheet format and processed to determine the % inhibition compared to the controls in each plate. The IC50 was calculated using Graphpad PRISM 7 software.

All designed compounds were screened at a fixed concentration of 10 PM. The H and the F compounds were proven to be ineffective. Only compounds H4 and F4 showed about 30% inhibition. Also, compounds H11 and H13 showed about 25% inhibition. Compounds MO1, MO9 and MO16 were synthesized and tested in the literature by Kravchenko et al. [69] and they were used in this study as internal controls. The molecules that accomplished higher than 80% inhibition were promoted to the IC₅₀ determination assays. IC₅₀ by definition is half maximal inhibitory concentration (IC₅₀) which is a measure of the effectiveness of a substance in blocking a specific biological or biochemical function. This quantitative measure indicates how much of a particular drug or other substance (inhibitor) is needed to inhibit a given biological process by half. The values are typically expressed as molar concentration. A lower IC50 reflects a higher potency and a higher IC₅₀ value is correlated to a lower potency. To determine IC50 assays, a total of 7 concentrations were tested for each compound. These concentrations were 50 μM, 15 μM, 3 μM, 0.6 μM (600 Nm), 0.12 μM (120 nM), 0.024 μM (24 nM) and 0.0048 μM (4.8 nM) respectively.

IC50 values as calculated by Graphpad PRISM 7 and the range of IC50 as estimated within 95% confidence limit for compounds that achieved more than 90% inhibition in the screening stage against caspases 3, 7 and 1.

Caspase 7 enzyme was found to be more stable, and the reaction equilibrium was attained within 30 to 40 minutes range while the reaction was faster with caspase 3 and the equilibrium was reached within 10 to 15 minutes. The compounds with high potency and efficacy against both caspase 3 and caspase 7 have shown more sigmoidal pattern in the dose response curve.

### Caspase 3 Enzyme Inhibitor Assay and Discussion

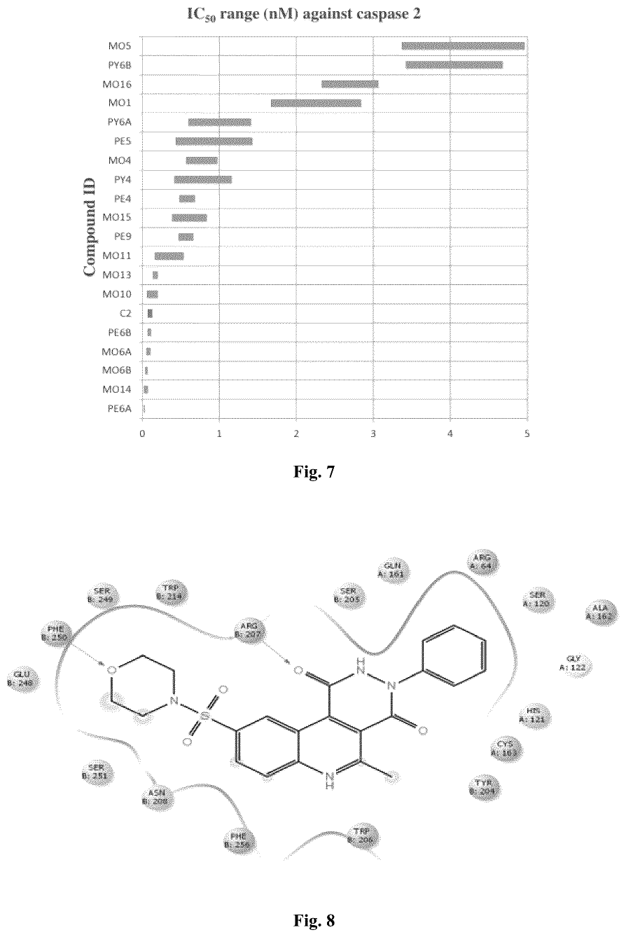

The calculated range of IC50 within 95% confidence intervals for each tested compound was demonstrated in FIG. 7. The smaller the range, the more accurate the results are and the broader this range is, the less accurate the results are. In view of FIG. 7, a narrower range was always associated with potent compounds like compound PE6A and a broader range was associated with less potent inhibitors like MO5. Compounds PE6A, MO6B and MO14 were shown to be statistically different and more potent than compound C2. On the other hand, compounds MO6A, MO10, MO13 and PE6B and showed a narrow range of IC50 but they were not statistically different from the control compound C2. Furthermore, there was no statistical difference between the mean values of each compound in this category.

Compound MO11 showed a broader range of IC50 and it was considered less potent than compound C2 and less potent than the previous two groups. Compounds PE9, MO15, PE4 and MO4 showed a relatively broader range than previous compounds and statistical difference compared to compound C2. These compounds are considered to be less potent than compound C2. Furthermore, there was no statistical difference between the mean values of each compound in this category.

Compounds PY4, PE5 and PY6A were shown to have a broader range of IC50 and the results showed a statistical difference between these compounds and compound C2. The potency of these compounds was about 5 folds less potent than the control compound C2. Furthermore, there was no statistical difference between the mean values of each compound in this category. Compounds MO1 and MO16 showed a much broader range of IC50 and they were about 20 folds less potent than compound C2. Furthermore, there was no statistical difference between the mean values of each compound in this category. Compounds MO5 and PY6B came last with the broadest range of IC50. These compounds were about 40 times less potent than the control compound C2. Furthermore, there was no statistical difference between the mean values of each compound in this category.

The IC50 of compound C2 against caspase 3 enzyme was determined to be 95.5 nM. In view of the IC50 assay results, the most potent inhibitors were PE6A, MO14, MO6B, MO6A and PE6B respectively.

The IC50 values were calculated to be between 20 to 100 nM which indicates that they are more potent than the selected positive control (compound C2). However, compound MO14 did not show the regular sigmoidal pattern but the plot showed some kind of a linear relationship. Compounds 6A and 6B share the same formula of 1,4-dioxo-5-methyl2,3,4-trihydro-1H-pyridazino[4,5-c]quinoline that are mainly modified at positions 2/3 and 9.

MO6A and MO6B compounds contain morpholine sulfonamide group at position 9 while PE6A and PE6B compounds contain piperidine sulfonamide group at position 9 of the molecular scaffold. FIGS. 57 through 60 demonstrate the intermolecular interactions of compound MO6A, MO6B, PE6A and PE6B quinoline into the active site of caspase 3 enzyme (obtained from Maestro 10.4).

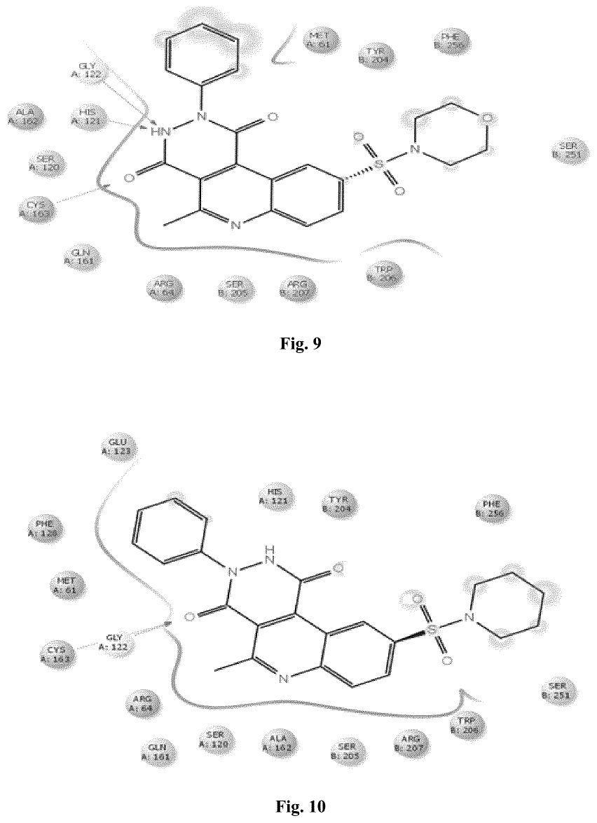

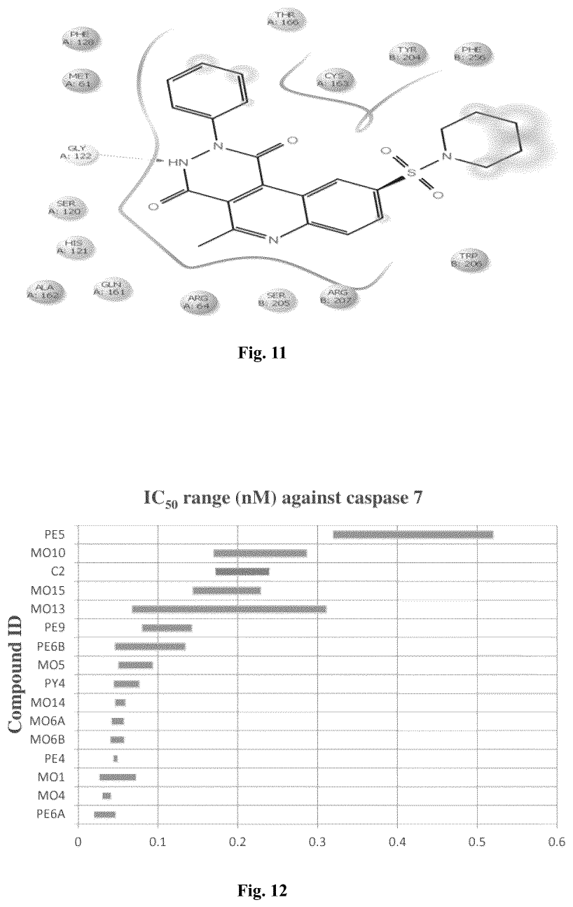

The most frequent interaction in FIGS. 8 to 11 is the hydrogen bond between the carbonyl group of the molecular scaffold and residues Gly122 and His121. Furthermore, it was noticed that the pyridazino ring of the compounds is constantly located in close proximity to both Cys163 and His121. In addition, the molecules seem to fit perfectly inside the hydrophobic grove of the enzyme via multiple hydrophobic interactions with the residues Tyr204, Trp206 and Phe256. Compounds MO10, MO13 and MO11 came next with a range of IC 50 values between 100 nM to 300 nM which makes these molecules a little less potent than the standard control compound C2. These compounds have substituents of C1, CH3 and I at the para position of the phenyl group located at position 2 of the molecular scaffold. Van der Waals radius for these substituents are 175 μm, 150 μm and 198 μm as calculated by Spartan 10®. Van der Waals is defined as half of the distance between the closest approaches of two non-bonded atoms of a given element. These substituents seem to be close in size and seem to be sterically favorable in this position especially with the residues Met61, Phe128 and Glu123.

The following compounds were not considered good inhibitors because their IC50 values were 5 to 10 times higher than the determined IC50 of the positive control (compound C2). Compounds PE9, MO15, PE4, PY4, MO4 and PY6A came next in line with IC50 values ranging between 550 nM and 1 μ1M. MO1 and MO16 were found to have high IC50 values (between 2 and 3 μ1M). Lastly, compounds PY6B and MO5 had calculated IC50 values of about 4 μ1M.

### Caspase 7 Enzyme Inhibitor Assay and Discussion

Compounds that accomplished the highest docking scores and were proven to have high activity and potency against caspase 3 enzyme were screened at a fixed concentration of 10 μM for anti-caspase 7 activities. The molecules that accomplished higher than 90% inhibition were promoted to the IC50 determination assay. To determine IC50 assays, a total of 7 concentrations were tested for each compound. These concentrations were 50 μM, 15 μM, 3 μM, 0.6 μM (600 Nm), 0.12 μM (120 nM), 0.024 μM (24 nM) and 0.0048 μM (4.8 nM) respectively.

Compounds PE6A, MO4, MO14, PE4, MO6A and MO6B were all within the same narrow range of IC50 with PE6A being the most potent inhibitor. A statistical significance was observed between these compounds and the range of IC50 of compound C2. These compounds were significantly more potent than compound C2 against caspase 7 enzyme. Furthermore, there was no clear statistical difference between the mean values of each compound in this category.

Compounds MO1, MO5 and PY4 were shown to have a broader range of IC50 than the first group but still more potent than compound C2. Furthermore, there was no clear statistical difference between the mean values of each compound in this category. Compounds PE9 and PE6B were shown to have a broader range of IC50 than the previous groups but still more potent than compound C2. Furthermore, there was no clear statistical difference between the mean values of each compound in this category. Compounds MO10 and MO15 were proven to have a similar range of IC50 which implied a similar potency to compound C2. However, there was no clear statistical difference between the mean values of each compound in this category. Compound PE5 was found to have the broadest range of IC50 and it was proven to be significantly less potent than compound C2.

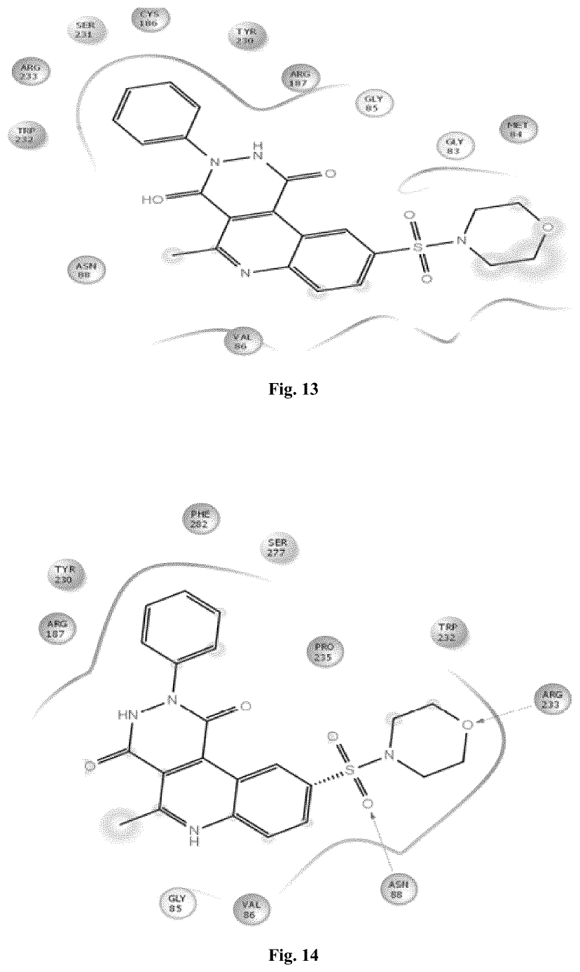

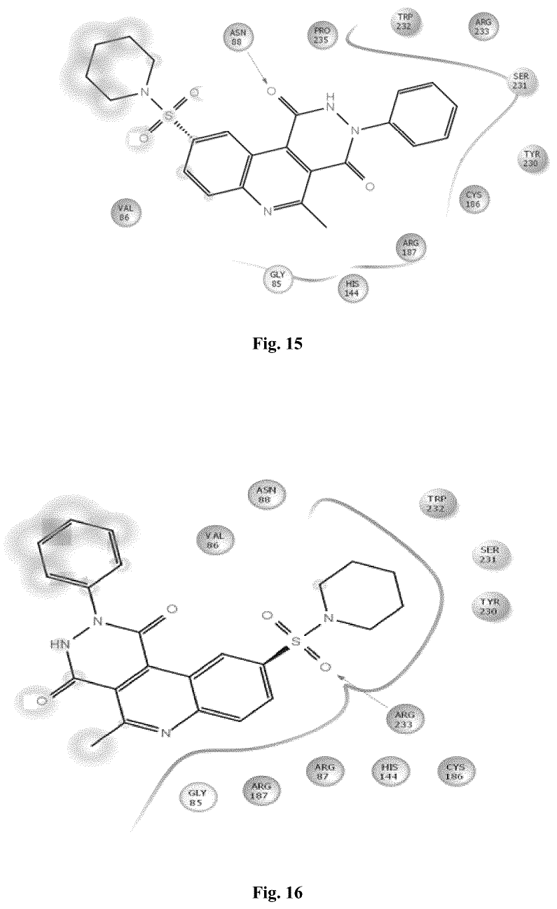

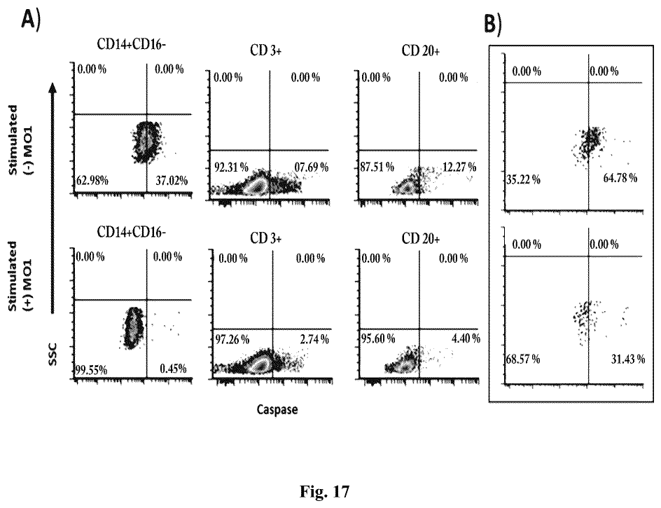

Compound MO13 accomplished the broadest range of IC50 values with a statistical significance that may appear to be similar to the positive control compound C2. Also, compound PE5 showed a broad range of IC50 values but it was less potent than the positive control C2. The IC50 of the positive control compound C2 against caspase 7 was determined to be 203 nM. In view of the IC50 assay results, the most potent inhibitors were PE6A, MO4, MO1, PE4, MO6B, MO6A and PE6B respectively. The IC50 values were calculated to be between 30 to 50 nM which indicates that they are a lot more potent than the selected positive control (compound C2). Compounds 6A and 6B share the same formula of 1,4dioxo-5-methyl-2,3,4-trihydro-1H-pyridazino[4,5-c]quinoline that are mainly modified at positions 2/3 and 9. MO6A and MO6B compounds contain morpholine sulfonamide group at position 9 while PE6A and PE6B compounds contain piperidine sulfonamide group at position 9 of the molecular scaffold. However, PE6B did not show the normal sigmoidal curve in its dose response curve and was less potent than the other three compounds. FIGS. 13 to 16 demonstrate the intermolecular interactions of compound MO6A, MO6B, PE6A and PE6B quinoline into the active site of caspase 7 enzymes (obtained from Maestro 10.4).

The most frequent interaction in FIGS. 62 through 65 is the hydrogen bond between the carbonyl group of the molecular scaffold and residues Gly85 and Arg187. Also, hydrogen bonding was observed occasionally between the inhibitors and Asn88 and Arg 233. Furthermore, it was noticed that the pyridazino ring of the compounds is constantly located at an average distance of about 3-5 Å from the active cysteine residue Cys186. In addition, the molecules seem to fit exactly inside the hydrophobic grove of the enzyme via multiple hydrophobic interactions with the residues Tyr230, Trp232 and Phe282. These molecules were found more potent than the standard positive control inhibitor (compound C2).

Compounds MO4, PE4 and PY4 were found to be 4 times more potent against caspase 7 compared to compound C2 with MO4 being the most potent inhibitor of them. Compounds MO4 and PE4 were very close in potency but PY4 was less potent than both of them. These compounds share the common feature of having a sulfonamide moiety at position 8 and a 2-methylquinoline-a-yl ring at position 2 of the traditional molecular scaffold. This is because of the size of the ring system of the sulfonamide at position 8 of the molecular scaffold. MO4 and PE4 share the same ring size which is a 6 membered ring while PY4 contains a 5 membered (pyrrolidine ring). These molecules were found more potent than the standard positive control inhibitor (compound C2) as well. The most common interaction observed between the inhibitor and the active site is the hydrogen bonding with residues Arg187 and Asn88. Also, strong hydrophobic interactions between the inhibitors and the hydrophobic grove were observed especially with residues Tyr230, Trp232 and Pro235.

Compound MO1 showed a high potency against caspase 7 enzyme (IC50=43.77 nM). Hydrogen bonding was demonstrated with Arg233 and the phenyl group at position 2 of the traditional molecular scaffold does fit the hydrophobic grove of the active site.

Compound MO14 showed a high potency against caspase 7 (IC50=52.2 nM) which was 4 times more potent than the positive control. Many hydrogen bonds were observed with the residues Gly85 and Arg187. Also, the methoxy group seems to create a favorable polar environment with the residues Ser231 and Arg233. Furthermore, the 4 methoxyphenyl group fits the hydrophobic grove of the enzyme. Compound PE9 showed a high potency against caspase 7 (IC50=106.8 nM) compared to the positive control. Many hydrogen bonds were observed with the residues Gly85, Arg187 and Ser231.

Compound MO13 was proven to be a little more potent than the positive control compound (IC50=144.8 nM). Hydrogen bonding was observed with the residues Arg 187 and Gly85. Furthermore, the 4-methylphenyl group at position 2 of the molecular scaffold seems to reinforce the hydrophobic interactions with the hydrophobic grove of the enzyme. Compound MO10 and MO15 seem to have a potency that is close to the potency of the positive control compound C2. The IC50 of these compounds were within 10% of the IC50 of compound C2. Hydrogen bonding was observed with the residues Arg 187 and Gly85. Furthermore, the 4-chlorophenyl group and 4-[N,N-dimethylaminophenyl]group at position 2 of the molecular scaffold seems to reinforce the hydrophobic interactions with the hydrophobic grove of the enzyme but since the 4-[N,N-dimethylaminophenyl]group is more polar, this leads to a slightly unfavorable impact on the hydrophobic grove.

Compounds MO5 and PE5 showed a big difference in their IC50 values. MO5 showed an IC50 of 68.6 nM while PE5 showed 407.7 nM. This difference in potency might be related to the favorable polar interaction between residue Asn88 and the oxygen atom of the morpholine group of the sulfonamide moiety at position 8 of the molecular scaffold of MO5. This interaction did not exist in case of the piperidine ring of PE5. Both MO5 and PE5 showed a hydrogen bond between the carbonyl group and the residue Asn88.

Comparative analysis: Compounds PE6A, PE6B, MO6A, MO6B, MO14 and MO13 were shown individually to have almost equal potency against both enzymes (caspase 3 and caspase7). Compounds PY4, PE4, MO4, PE5, MO5, PE9, MO1 and MO15 were found to have at least 5 times much higher potency against caspase 7 compared to their potencies against caspase 3. MO10 was the only compound that showed a higher potency against caspase 3 compared to its potency against caspase 7.

Table 4 below summarizes IC50 of different compounds against caspase 1,3 and 7

| | IC₅₀ (nM) against | IC₅₀ (nM) against | IC₅₀ (nM) against |
|---|---|---|---|
| ID | Caspase 3 | Caspase 7 | Caspase 1 |
| PY4 | 693.8 | 58.29 | 137 |
| PE4 | 572.8 | 46.7 | 274 |
| PE5 | 784.9 | 407.7 | 261 |
| PE6A | 23.54 | 30.15 | 379 |
| PE6B | 86.91 | 78.48 | 112 |
| PE9 | 557.1 | 106.8 | 165 |
| MO6A | 74.94 | 48.9 | 228 |
| MO6B | 49.09 | 48.21 | 167 |
| MO1 | 2,177 | 43.77 | 121 |
| MO4 | 742.4 | 35.24 | 178 |
| MO5 | 4,089 | 68.58 | 334 |
| MO10 | 107.8 | 220.6 | 633 |
| MO13 | 164.3 | 144.8 | 361 |
| MO14 | 37.18 | 52.2 | 342 |
| MO15 | 567.2 | 181.3 | 393 |

The results indicate that compounds with the 6 membered ring pharmacophore are potent non-selective inhibitors. Compound MO13 showed a similar behavior but less potency than the first group of compounds. The 2 methylquinoline-yl ring at position 2 of the traditional molecular scaffold of compounds PY4, PE4 and MO4 might be the reason behind the differential activity. This differential activity might be related to the nature of the quinoline ring that seem to anchor the inhibitor in the hydrophobic grove of caspase 7 active site more prominently compared to the hydrophobic grove of caspase 3 active site. Compounds PE9, MO1 and MO15 showed a similar behavior with a higher relative potency against caspase 7 enzyme compared to caspase 3. The only compounds that showed a higher potency against caspase 3 versus caspase 7 is compound MO10. This could be contributed to the steric size of the Cl atom (MO10) at the para phenyl group at position 2 of the molecular scaffold or the relative polarity of these groups that potentiates more favorable interactions with the active site of caspase 3 compared to caspase 7.

### Example 6: Ex-Vivo Analysis

### PBMC Isolation

Freshly obtain peripheral blood mononuclear cells (PBMC) were separated from 2 mL of whole blood within 24 h of collection and diluted 1:1 with phosphate buffered saline pH 7.2 (PBS) (Thermo Fisher Scientific, Carlsbad, CA) using Lymphoprep (Stem cell Technologies, Cambridge, MA) and Accuspin tubes (Sigma-Aldrich, St. Louis, MO) as per manufactures directions. PBMCs' were washed in PBS and resuspended in PBS.

### Flow Cytometry

Apoptosis and pyroptosis were measured by flow cytometry using fluorescent-labeled inhibitors of caspase 1, 3/7, 8 and 10 (FLICA; Immunochemistry Technologies, Minneapolis, MN). PBMCs were either non-stimulated or stimulated with 10 μM nigericin (Caspase 1) or 1 μM staurosporine (Caspase 3/7, 8, 10) or for 2 hours. FAM-FLICA probes specific for caspase-1, 3/7, 8, 10 were added to 50 μl PBMC and incubated for 1 h at 37° C. Cells were subsequently washed and stained with a cocktail of antibodies consisting of CD45 EF506 [HI30], CD16 SB436 [3G8], CD14 PerCP-Cy5.5 [M5E2], CD3 APC [UCHT1], CD4 PE [SK3], CD8 R718 [SK1], CD20 PE [2H7] and Viability Dye 780 (Thermo Fisher Scientific, Carlsbad, CA) for 30 minutes at 4° C. Cells were washed prior to acquisition on a 3 laser ThermoFisher Attune NxT. Performance beads (Thermo Fisher Scientific, Carlsbad, CA) were acquired daily to ensure consistent performance of the Attune NxT. Denovo FCS Express v7 clinical edition (De Novo Software, Pasadena, CA) was used for flow cytometric analyses.

### Gating Strategy for T, B and Monocyte Cells

Monocytes were identified by a standard gating strategy: singlets (FSC-A/FSC—H); viability gate (Viability Dye 780−); (CD45+(CD45/SSC plot), CD14, and CD16 to identify classical (CD14+CD16−), intermediate (CD14+CD16+) and nonclassical (CD14-CD16+) monocytes. Lymphocytes were identified using the following gating schematic: singlets (FSC-A/FSC—H); CD45+(CD45/SSC plot); viability gate (Viability Dye 780−). T cells were identified by CD45+CD3+(CD3/SSC plot); CD4+ and CD8+ T cells (CD4/CD8 CD3+ gating). From the lymphocyte gating plots, B cells were identified as: CD45+; CD20+(CD20/SSC plot). Active caspase within the identified cell populations was determined by FAM-FLICA probes specific for caspase-1, 3/7, 8, 10. Surface PAI-1 was identified with PAI-1 Corelite 488 (ProteinTech).

### PBMC Treatment

Prior to PBMC purification whole blood was either untreated or treated with nattokinase (10 g/ml) or serratiopeptidase (100 μg/ml) for 1 hour at 37° C. PBMCs were either untreated or treated with Nattokinase (40 μg/ml) or serratiopeptidase (0.4 mg/ml) during the 2-hour stimulation with either nigericin or staurosporine as indicated previously.

### Differential Structure Activity Relationship

The 6 membered pharmacophore ring (pyridazino) in compounds broadened the spectrum of activity and enhanced the potency.

The presence of a heterocyclic group like the 4-pyridyl group at position 2 of the traditional molecular scaffold enhanced the potency against caspase 7 compared to caspase 3.

The presence of a quinoline ring at position 2 of the traditional molecular scaffold enhanced the selectivity of the inhibitor against caspase 7 enzyme versus caspase enzyme.

4. The presence of ethynyl group at the met a position of the phenyl ring at position 2 of the traditional molecular scaffold enhanced the selectivity against caspase 7 compared to caspase 3.

5. Presence of a 6 membered ring (piperidine or morpholine) at the sulfonamide moiety endorsed the activity more than the 5 membered ring (pyrrolidine) at position 8 of the traditional molecular scaffold. However, there was no significant difference between using piperidine and morpholine.

6. A substituent at the para position of the phenyl group at position 2 is very critical for the activity and potency of the inhibitor. A methyl group or chlorine seems to lower the selectivity of the inhibitor, a Fluorine atom seems to ruin the activity. However, different groups with various sizes may have differential impact on the selectivity of the inhibitor.

### REFERENCES

All references, including granted patents and patent application publications, referred herein are incorporated herein by reference in their entirety.
1) Hengartner, M. O., “The biochemistry of apoptosis”. Nature 2000, 407 (6805), 770-6.
2) Reed, J., “Apoptosis based therapies”. Nature Reviews Drug Discovery 2002, 1, 111-121.
3) MacBride, C.; McPhail, L.; Stevens, J., “Emerging therapeutic targets in caspase-dependent disease”. Emerging Therapeutic Targets 1999, 3, 391-411.
4) Fuentes-Prior, P.; Salvesen, G. S., “The protein structures that shape Caspase activity, specificity, activation, and inhibition”. Biochem. J. 2004, 384, 201-232.
5) Lavrik, I. N.; Golks, A.; Krammer, P. H., “Caspases: pharmacological manipulation of cell death”. The Journal of clinical investigation 2005, 115 (10), 2665-72.
6) Proskuryakov, S. Y.; Konoplyannikov, A. G.; Gabai, V. L., “Necrosis: a specific form of programmed cell death?”. Experimental cell research 2003, 283 (1), 1-16.
7) ADDIN E N. REFLIST Boatright, K. M.; Salvesen, G. S., “Mechanisms of Caspase activation”. Current opinion in cell biology 2003, 15 (6), 725-31.
8) Earnshaw, W. C.; Martins, L. M.; Kaufmann, S. H., “Mammalian Caspases: structure, activation, substrates, and functions during apoptosis”. Annual review of biochemistry 1999, 68, 383-424.
9) Methot, N.; Vaillancourt, J. P.; Huang, J.; Colucci, J.; Han, Y.; Menard, S.; Zamboni, R.; Toulmond, S.; Nicholson, D. W.; Roy, S., “A Caspase active site probe reveals high fractional inhibition needed to block DNA fragmentation”. The Journal of biological chemistry 2004, 279 (27), 27905-14.
10) Li, J.; Yuan, J., “Caspases in apoptosis and beyond”. Oncogene 2008, 27 (48), 6194-206.
11) Shi, Y., “Mechanisms of Caspase activation and inhibition during apoptosis”. Molecular cell 2002, 9 (3), 459-70.
12) Shi, Y., “Activation of initiator Caspases: History, hypotheses, and perspectives”. Journal of Cancer Molecules 2005, 1 (1), 9-18.
13) Riedl, S. J.; Shi, Y., “Molecular mechanisms of Caspase regulation during apoptosis”. Nature reviews. Molecular cell biology 2004, 5 (11), 897-907.
14) Lavrik, I. N.; Golks, A.; Krammer, P. H., “Caspases: pharmacological manipulation of cell death”. The Journal of clinical investigation 2005, 115 (10), 2665-72.
15) Fuentes-Prior, P.; Salvesen, G. S., “The protein structures that shape Caspase activity, specificity, activation and inhibition”. The Biochemical journal 2004, 384 (Pt 2), 201-32.
16) Parrish, A. B.; Freel, C. D.; Kornbluth, S., “Cellular mechanisms controlling Caspase activation and function”. Cold Spring Harbor perspectives in biology 2013, 5 (6), a008672.
17) Wei, Y.; Fox, T.; Chambers, S. P.; Sintchak, J.; Coll, J. T.; Golec, J. M.; Swenson, L.; Wilson, K. P.; Charifson, P. S., “The structures of Caspases-1, -3, -7 and -8 reveal the basis for substrate and inhibitor selectivity”. Chemistry and biology 2000, 7 (6), 423-32.
18) Stennicke, H. R.; Salvesen, G. S., “Biochemical characteristics of Caspases-3, -6, -7, and -8”. The Journal of biological chemistry 1997, 272 (41), 25719-23.
19) Agniswamy, J.; Fang, B.; Weber, I. T., “Plasticity of S2-S4 specificity pockets of executioner Caspase 7 revealed by structural and kinetic analysis”. The FEBS journal 2007, 274 (18), 4752-65.
20) Chai, J.; Shiozaki, E.; Srinivasula, S. M.; Wu, Q.; Datta, P.; Alnemri, E. S.; Shi, Y., “Structural basis of Caspase 7 inhibition by XIAP”. Cell 2001, 104 (5), 769-80.
21) Walsh, J. G.; Cullen, S. P.; Sheridan, C.; Luthi, A. U.; Gerner, C.; Martin, S. J., “Executioner Caspase 3 and Caspase 7 are functionally distinct proteases”. Proceedings of the National Academy of Sciences of the United States of America 2008, 105 (35), 12815-9.
22) Lamkanfi, M.; Kanneganti, T. D., “Caspase 7: a protease involved in apoptosis and inflammation”. The international journal of biochemistry and cell biology 2010, 42 (1), 21-4.
23) Chereau, D.; Kodandapani, L.; Thomaselli, K. J.; Spada, A. P.; Wu, J. C., “Structural and functional analysis of Caspase active site”. Biochemistry 2003, 42 (14), 4151-60.
24) McLuskey, K.; Mottram, J. C., “Comparative structural analysis of the Caspase family with other clan CD cysteine peptidases”. The Biochemical journal 2015, 466 (2), 219-32.
25) Miscione, G. P.; Calvaresi, M.; Bottoni, A., “Computational evidence for the catalytic mechanism of Caspase 7”. A DFT investigation. The journal of physical chemistry. B 2010, 114 (13), 4637-45.
26) Sulpizi, M., Laio., A.; Vande Vondele. J.; Cattaneo, A.; Rothlisberger, U.; Carloni, P., “Reaction mechanism of Caspases: insights from QM/MM Car-Parrinello simulations”. Protein. 2003, 52(2), 212-224.
27) Favaloro, B.; Allocati, N.; Graziano, V.; Di Ilio, C.; De Laurenzi, V., “Role of apoptosis in disease”. Aging 2012, 4 (5), 330-49.
28) McIlwain, D. R.; Berger, T.; Mak, T. W., “Caspase functions in cell death and disease”. Cold Spring Harbor perspectives in biology 2013, 5 (4), a008656.
29) Condorelli, G.; Roncarati, R.; Ross, J., Jr.; Pisani, A.; Stassi, G.; Todaro, M.; Trocha, S.; Drusco, A.; Gu, Y.; Russo, M. A.; Frati, G.; Jones, S. P.; Lefer, D. J.; Napoli, C.; Croce, C. M., “Heart-targeted overexpression of Caspase3 in mice increases infarct size and depresses cardiac function”. Proceedings of the National Academy of Sciences of the United States of America 2001, 98 (17), 9977-82.
30) Yang, B.; Ye, D.; Wang, Y., “Caspase 3 as a therapeutic target for heart failure” Expert Opin Ther Targets. 2013, 17(3), 255-63.
31) Ruetten, H.; Badorff, C.; Ihling, C.; Zeiher, A. M.; Dimmeler, S., “Inhibition of Caspase 3 improves contractile recovery of stunned myocardium, independent of apoptosis-inhibitory effects”. Journal of the American College of Cardiology 2001, 38 (7), 2063-70.
32) Kim, E. S.; Yoo, S.; Yi, K. Y.; Lee, S.; Noh, J. S.; Jung, Y. C.; Kim, E.; Jeong, N., “Design, Synthesis and Biological Evaluation of Nonpeptide Caspase 3 Inhibitors”. Bulletin of Korean Chemical Society 2002, 23 (7), 1003-1011.
33) Jana, K.; Banerji, B.; Parida, P., “Caspases: A Potential Therapeutic Targets in the Treatment of Alzheimer's Disease”. Translational Medicine 2013, S2 (003) doi:10.4172/2161-1025.
34) Klafki, H. W.; Staufenbiel, M.; Kornhuber, J.; Wiltfang, J., “Therapeutic approaches to Alzheimer's disease”. Brain: a journal of neurology 2006,129 (Pt 11), 2840-55.
35) Hartmann, A.; Hunot, S.; Michel, P. P.; Muriel, M. P.; Vyas, S.; Faucheux, B. A.; Mouatt-Prigent, A.; Turmel, H.; Srinivasan, A.; Ruberg, M.; Evan, G. I.; Agid, Y.; Hirsch, E. C., “Caspase 3: A vulnerability factor and final effector in apoptotic death of dopaminergic neurons in Parkinson's disease”. Proceedings of the National Academy of Sciences of the United States of America 2000, 97 (6), 2875-80.
36) Khan, S.; Ahmad, K.; Alshammari, E. M.; Adnan, M.; Baig, M. H.; Lohani, M.; Somvanshi, P.; Haque, S., “Implication of Caspase 3 as a Common Therapeutic Target for Multineurodegenerative Disorders and Its Inhibition Using Nonpeptidyl Natural Compounds”. BioMed research international 2015, 2015, 379817.
37) Han, B. H.; Xu, D.; Choi, J.; Han, Y.; Xanthoudakis, S.; Roy, S.; Tam, J.; Vaillancourt, J.; Colucci, J.; Siman, R.; Giroux, A.; Robertson, G. S.; Zamboni, R.; Nicholson, D. W.; Holtzman, D. M., “Selective, reversible Caspase 3 inhibitor is neuroprotective and reveals distinct pathways of cell death after neonatal hypoxic-ischemic brain injury”. The Journal of biological chemistry 2002, 277 (33), 30128-36.
38) Rideout, H. J.; Stefanis, L., “Caspase inhibition: a potential therapeutic strategy in neurological diseases”. Histology and histopathology 2001, 16 (3), 895-908.
39) Jo, J.; Whitcomb, D. J.; Olsen, K. M.; Kerrigan, T. L.; Lo, S. C.; Bru-Mercier, G.; Dickinson, B.; Scullion, S.; Sheng, M.; Collingridge, G.; Cho, K., “Aß(1-42) inhibition of LTP is mediated by a signaling pathway involving Caspase-3, Akt1 and GSK-3beta” Nature Neuroscience 2011, 14, 545-547.
40) Uetsuki, T.; Takemoto, K.; Nishimura, I.; Okamoto, M.; Niinobe, M.; Momoi, T.; Miura, M.; Yoshikawa, K., “Activation of neuronal Caspase 3 by intracellular accumulation of wild-type Alzheimer amyloid precursor protein”. The Journal of neuroscience: the official journal of the Society for Neuroscience 1999, 19 (16), 6955-64.
41) Wang, K., “Molecular mechanisms of hepatic apoptosis”. Cell Death and Disease 2014, 5 (996).
42) Wang, K.; Liu, B., “Pathophysiological Significance of Hepatic Apoptosis”. ISRN Hepatology 2013, 2013, Article ID 740149.
43) Masuokal, H.; Guicciardi, M.; Gores, G., “Caspase inhibitors for the treatment of hepatitis C”. NIH public Access 2009, 13 (3), 467-475.
44) Dean, E. J.; Ranson, M.; Blackhall, F.; Holt, S. V.; Dive, C., “Novel therapeutic targets in lung cancer: Inhibitor of apoptosis proteins from laboratory to clinic”. Cancer treatment reviews 2007, 33 (2), 203-12.
45) Kim, K. W.; Moretti, L.; Lu, B., “M867, a novel selective inhibitor of Caspase 3 enhances cell death and extends tumor growth delay in irradiated lung cancer models”. PloS one 2008, 3 (5), e2275.
46) Wei, Y.; Fan, T.; Yu, M., “Inhibitor of apoptosis proteins and apoptosis”. Acta biochimica et biophysica Sinica 2008, 40 (4), 278-88.
47) Vucic, D.; Fairbrother, W. J., “The inhibitor of apoptosis proteins as therapeutic targets in cancer”. Clinical cancer research: an official journal of the American Association for Cancer Research 2007, 13 (20), 5995-6000.
48) Tsujimoto, Y., “Role of Bcl-2 family proteins in apoptosis: apoptosomes or mitochondria?”. Genes Cells 1998, 3 (11), 697-707.
49) Sulpizi, M.; Rothlisberger, U.; Carloni, P., “Molecular dynamics studies of Caspase 3”. Biophysical journal 2003, 84 (4), 2207-15.
50) Fuentes-Prior, P.; Salvesen, G. S., “The protein structures that shape Caspase activity, specificity, activation and inhibition”. The Biochemical journal 2004, 384 (Pt 2), 201-32.
51) Isabel, E.; Black, W. C.; Bayly, C. I.; Grimm, E. L.; Janes, M. K.; McKay, D. J.; Nicholson, D. W.; Rasper, D. M.; Renaud, J.; Roy, S.; Tam, J.; Thornberry, N. A.; Vaillancourt, J. P.; Xanthoudakis, S.; Zamboni, R., “Nicotinyl aspartyl ketones as inhibitors of Caspase 3”. Bioorganic and medicinal chemistry letters 2003, 13 (13), 2137-40.
52) Caserta, T. M.; Smith, A. N.; Gultice, A. D.; Reedy, M. A.; Brown, T. L., “Q-VD-OPH, a broad spectrum Caspase inhibitor with potent antiapoptotic properties”. Apoptosis 2003, 8, 345-352.
53) Allen, D. A.; Pham, P.; Choong, I. C.; Fahr, B.; Burdett, M. T.; Lew, W.; DeLano, W. L.; Gordon, E. M.; Lam, J. W.; O'Brien, T.; Lee, D., “Identification of potent and novel small-molecule inhibitors of Caspase 3”. Bioorganic and medicinal chemistry letters 2003, 13 (21), 3651-5.
54) Erlanson, D. A.; Lam, J.; Wiesman, C.; Luong, T.; Simmons, R.; DeLano, W.; Choong, I.; Burdett, M.; Flanagan, M.; Lee, D.; Gordon, E.; O'Brien, T., “In situ assembly of enzyme inhibitors using extended tethering”. Nature Biochemistry 2003, 21(3), 308-314.
55) Isabel, E.; Black, W. C.; Bayly, C. I.; Grimm, E. L.; Janes, M. K.; McKay, D. J.; Nicholson, D. W.; Rasper, D. M.; Renaud, J.; Roy, S.; Tam, J.; Thornberry, N. A.; Vaillancourt, J. P.; Xanthoudakis, S.; Zamboni, R., “Nicotinyl aspartyl ketones as inhibitors of Caspase 3”. Bioorganic and medicinal chemistry letters 2003, 13 (13), 2137-40.
56) Boivin, W.; Cooper, D.; Hiebert, P.; Granville, D., “Intracellular versus extracellular grandzyme B in immunity and disease: challenging the dogma”. Laboratory Investigation 2009, 89(11), 1195-1220.
57) Rousalova, I.; Krepela, E., “Grandzyme B-induced apoptosis in cancer cells and its regulation (Review)”. International Journal of Oncology 2010, 37(6), 1361-78.
58) Willoughby, C. A.; Bull, H. G.; Garcia-Calvo, M.; Jiang, J.; Chapman, K. T.; Thornberry, N. A., “Discovery of potent, selective human granzyme B inhibitors that inhibit CTL mediated apoptosis”. Bioorganic and medicinal chemistry letters 2002, 12 (16), 2197-200.
59) Shin, S.; Sung, B. J.; Cho, Y. S.; Kim, H. J.; Ha, N. C.; Hwang, J. I.; Chung, C. W.; Jung, Y. K.; Oh, B. H., “An anti-apoptotic protein human survivin is a direct inhibitor of Caspase 3 and -7”. Biochemistry 2001, 40 (4), 1117-23.
60) Linton, S. D., “Caspase inhibitors: a pharmaceutical industry perspective”. Current topics in medicinal chemistry 2005, 5 (16), 1697-717.
61) Han, Y.; Giroux, A.; Colucci, J.; Bayly, C. I.; McKay, D. J.; Roy, S.; Xanthoudakis, S.; Vaillancourt, J.; Rasper, D. M.; Tam, J.; Tawa, P.; Nicholson, D. W.; Zamboni, R. J., “Novel pyrazinone mono-amides as potent and reversible Caspase 3 inhibitors”. Bioorganic and medicinal chemistry letters 2005, 15 (4), 1173-80.
62) Jin-liang, Z.; Yang-hong, G.; Chao-hui, G.; En-si, W.; Zhong-yuan, R.; Xue-xun, F.; jJi-dong, W., “Design, synthesis and molecular docking of a novel small-molecule inhibitor of Caspase 3”. Chemical Research in Chinese Universities 2010, 26 (2), 256-258.
63) Okun, I.; Malarchuk, S.; Dubrovskaya, E.; Khvat, A.; Tkachenko, S.; Kysil, V.; Ilyin, A.; Kravchenko, D.; Prossnitz, E. R.; Sklar, L.; Ivachtchenko, A., “Screening for Caspase 3 inhibitors: a new class of potent small-molecule inhibitors of Caspase 3”. Journal of biomolecular screening 2006, 11 (3), 277-85.
64) Chu, W.; Zhang, J.; Zeng, C.; Rothfuss, J.; Tu, Z.; Chu, Y.; Reichert, D. E.; Welch, M. J.; Mach, R. H., “N-benzoylisatin sulfonamide analogues as potent Caspase 3 inhibitors: synthesis, in vitro activity, and molecular modeling studies”. Journal of medicinal chemistry 2005, 48 (24), 7637-47.
65) Jiang, Y.; Hansen, T. V., “Isotin 1,2,3-triazoles as potent inhibitors against Caspase 3”. Bioorganic and medicinal chemistry letters 2011, 21 (6), 1626-9.
66) Kopka, K.; Faust, A.; Keul, P.; Wagner, S.; Breyholz, H. J.; Holtke, C.; Schober, O.; Schafers, M.; Levkau, B., “5-pyrrolidinylsulfonyl isotins as a potential tool for the molecular imaging of Caspases in apoptosis”. Journal of medicinal chemistry 2006, 49 (23), 6704-15.
67) Ivachtchenko, A. V.; Kobak, V. V.; Zolotarev, D. A.; Boksha, L. V.; Trifilenkov, A. S.; Ugoleva, D. M., “New Scaffolds for Combinatorial Synthesis. 1. 5-Sulfamoylisatins and Their Reactions with 1,2-Diamines”. Journal of Combinational Chemistry 2002, 4 (5), 419-428.
68) Kravchenko, D. V.; Kysil, V. V.; Ilyn, A. P.; Tkachenko, S. E.; Maliarchouk, S.; Okun, I. M.; Ivachtchenko, A. V., “1,3-Dioxo-4-methyl-2,3-dihydro-1H-pyrrolo[3,4-c]quinolines as potent Caspase 3 inhibitors”. Bioorganic and medicinal chemistry letters 2005, 15 (7), 1841-5.
69) Kravchenko, D. V.; Kuzovkova, Y. A.; Kysil, V. M.; Tkachenko, S. E.; Maliarchouk, S.; Okun, I. M.; Balakin, K. V.; Ivachtchenko, A. V., “Synthesis and structure-activity relationship of 4-substituted 2-(2-acetyloxyethyl)-8-(morpholine-4-sulfonyl)pyrrolo[3,4-c]quinoline-1,3-diones as potent Caspase 3 inhibitors”. Journal of medicinal chemistry 2005, 48 (11), 3680-3.
70) Becker, J. W.; Rotonda, J.; Soisson, S. M.; Aspiotis, R.; Bayly, C.; Francoeur, S.; Gallant, M.; Garcia-Calvo, M.; Giroux, A.; Grimm, E.; Han, Y.; McKay, D.; Nicholson, D. W.; Peterson, E.; Renaud, J.; Roy, S.; Thornberry, N.; Zamboni, R., “Reducing the peptidyl features of Caspase 3 inhibitors: a structural analysis”. Journal of medicinal chemistry 2004, 47 (10), 2466-74.
71) Wang, Y.; Guan, L.; Jia, S.; Tseng, B.; Drewe, J.; Cai, S. X., “Dipeptidyl aspartyl fluoromethyl ketones as potent Caspase inhibitors: peptidomimetic replacement of the P2 alpha-amino acid by a alpha-hydroxy acid”. Bioorganic and medicinal chemistry letters 2005, 15 (5), 1379-83.
72) Grimm, E. L.; Roy, B.; Aspiotis, R.; Bayly, C. I.; Nicholson, D. W.; Rasper, D. M.; Renaud, J.; Roy, S.; Tam, J.; Tawa, P.; Vaillancourt, J. P.; Xanthoudakis, S.; Zamboni, R. J., “Solid phase synthesis of selective Caspase 3 peptide inhibitors”. Bioorganic and medicinal chemistry 2004, 12 (5), 845-51.
73) Suzuki, Y.; Nakabayashi, Y.; Nakata, K.; Reed, J.; Takahashi, R. “X-linked inhibitor of apoptosis protein (XIAP) inhibits Caspase 3 and -7 in distinct modes” The Journal of Biological Chemistry 2001, 276, 27058-27063.
74) Sharma, S.; Basu, A.; Agrawal, R. K., “Pharmacophore modeling and docking studies on some nonpeptide-based Caspase 3 inhibitors”. BioMed research international 2013, 2013, 306081.
75) Sangshetti, J. N.; Zambare, A. S.; Shinde, D. B.; Gonjari, I., “Pfitzinger Reaction in Synthesis of Bioactive Compounds—A Review”. Mini-Review in Organic Chemistry 2014, 11 (2), 225-250.
76) Ivachtchenko, A. V.; Khvat, A. V.; Kobak, V. V.; Kysil, V. M.; Williams, C. T., “A new insight into the Pfitzinger reaction. A facile synthesis of 6-sulfamoylquinoline-4-carboxylic acids”. Tetrahedron Letters 2004, 45 (28), 5473-5476.
77) Flutsch, A.; Ackermann, R.; Schroeder, T.; Lukarska, M.; Hausammann, G.; Weinert, C.; Briand, C.; Grutter, M., “Combined inhibition of Caspase 3 and Caspase 7 by two highly selective DARPins slow a down cellular demise”. Journal of Biochemistry 2014, 461, 279-290.
78) Agniswamy, J.; Fang, B.; Weber, I. T., “Conformational similarity in the activation of Caspase 3 and -7 revealed by the unliganded and inhibited structures of Caspase 7”. Apoptosis: an international journal on programmed cell death 2009, 14 (10), 1135-44.
79) Scheer, J. M.; Romanowski, M. J.; Wells, J. A., “A common allosteric site and mechanism in Caspases”. Proceedings of the National Academy of Sciences of the United States of America 2006, 103 (20), 7595-600.
80) Morris, G. M., Huey, R., Lindstrom, W., Sanner, M. F., Belew, R. K., Goodsell, D. S. and Olson, A. J. “Autodock4 and AutoDockTools4: automated docking with selective receptor flexibility”. Journal of Computational Chemistry 2009, 16, 2785-91.
81) Huang, S. Y.; Zou, X., “An iterative knowledge-based scoring function to predict protein-ligand interactions: II. Validation of the scoring function”. Journal of computational chemistry 2006, 27 (15), 1876-82.
82) Wang, R.; Lai, L.; Wang, S., “Further Development and Validation of Empirical Scoring Functions for Structure-Based Binding Affinity Prediction”, Journal of Computer Aided Molecular Design 2002, 16(1), 11-26.
83) Lipinski, C. A.; Lombardo, F.; Dominy, B. W.; Feeney, P. J., “Experimental and computational approaches to estimate solubility and permeability in drug discovery and development settings”. Advanced drug delivery reviews 2001, 46 (1-3), 3-26.
84) Lee, D.; Long, S. A.; Adams, J. L.; Chan, G.; Vaidya, K. S.; Francis, T. A.; Kikly, K.; Winkler, J. D.; Sung, C. M.; Debouck, C.; Richardson, S.; Levy, M. A.; DeWolf, W. E., Jr.; Keller, P. M.; Tomaszek, T.; Head, M. S.; Ryan, M. D.; Haltiwanger, R. C.; Liang, P. H.; Janson, C. A.; McDevitt, P. J.; Johanson, K.; Concha, N. O.; Chan, W.; Abdel-Meguid, S. S.; Badger, A. M.; Lark, M. W.; Nadeau, D. P.; Suva, L. J.; Gowen, M.; Nuttall, M. E., “Potent and selective nonpeptide inhibitors of Caspases 3 and 7 inhibit apoptosis and maintain cell functionality”. The Journal of biological chemistry 2000, 275 (21), 16007-14.
85) Scott, C. W.; Sobotka-Briner, C.; Wilkins, D. E.; Jacobs, R. T.; Folmer, J. J.; Frazee, W. J.; Bhat, R. V.; Ghanekar, S. V.; Aharony, D., “Novel small molecule inhibitors of Caspase 3 block cellular and biochemical features of apoptosis”. The Journal of pharmacology and experimental therapeutics 2003, 304 (1), 433-40.
86) Chai, C. W.; Sobotka-Briner, C.; Wilkins, D. E.; Jacobs, R. T.; Folmer, J. J.; Frazee, J.; Wu, Q.; Shiozaki, E.; Srinivasula S. M.; Alnemri, E. S.; Shy, Y. “Crystal structure of a procaspase-7 zymogen: mechanisms of activation and substrate binding”. Cell 2001, 107 (3), 399-407.

1. A compound with the following formula I: 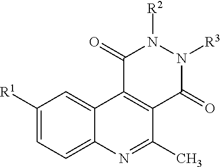
wherein “R¹”, “R²”, and “R³” comprise at least one of:
Hydrogen;
Hydroxyl;
Alkyl group;
Alkoxy group;
Acyloxy group;
Alkenyl group;
Alkynyl group;
Aromatic group;
Heterocyclic group;
or any salt, isomer, ester, or derivative thereof, for the modulation of caspase activity.

2. The compound, according to claim 1,
wherein “R¹” comprise at least one of the following:
Hydrogen;
Halogen;
Nitro group;
Aldehyde group;
Sulfhydryl group;
Carboxyl group;
Cyano group;
Amino groups: Nitrogen-containing groups comprising either primary (NH₂), secondary (NHR), or tertiary (NR₂), with R being any alkyl or aryl group,
Sulfonyl group (—SO₂R): whereas R is selected from hydrogen, an alkyl or aryl group;
or any salt, isomer, ester, or derivative thereof, for the modulation of caspase activity.

3. The compound, according to claim 2,
wherein the R group on the (—SO₂R) of “R¹” is a heterocyclic aliphatic or heterocyclic aromatic structure;
or any salt, isomer, ester, or derivative thereof, for the modulation of caspase activity.

4. The compound, according to claim 1,
wherein “R¹”, “R²”, or “R³” is an Alkyl group comprise at least one of the following:
Straight Alkyl group;
Branched Alkyl group;
Cyclic Alkyl group;
Heterocyclic aliphatic group;
or any salt, isomer, ester, or derivative thereof, for the modulation of caspase activity.

5. The compound, according to claim 4,
wherein the heterocyclic aliphatic group of “R¹” is a piperidine, pyrrolidine, morpholine, imidazolidine, pyrane, or any substituted moiety thereof,
or any salt, isomer, ester, or derivative thereof, for the modulation of caspase activity.

6. The compound, according to claim 1,
wherein “R¹”, “R²”, or “R³” is an aromatic group comprise at least one of:
Monocyclic aromatic group or any substituted moiety thereof;
Polycyclic aromatic group or any substituted moiety thereof;
or any salt, isomer, ester, or derivative thereof, for the modulation of caspase activity.

7. The compound, according to claim 6,
wherein “R²” or “R³” is an aromatic group comprise at least one five or six membered ring or any substituted moiety thereof;
or any salt, isomer, ester, or derivative thereof, for the modulation of caspase activity.

8. The compound, according to claim 7,
wherein “R²” and/or “R³” comprising an aromatic group comprising either:
a benzene ring, a pyridine, indole, a quinoline, isoquinoline; Imidazole; Pyrazole; Imidazoline; Pyrazoline; 1,2,3-Triazole; 1,2,4-Triazole; Pyridazine; Pyrimidine; Pyrazine; 1,2-Diazine; 1,3-Diazine; 1,4-Diazine; or any substituted moiety thereof;
or any salt, isomer, ester, or derivative thereof, for the modulation of caspase activity.

9. The compound, according to claim 1, with the following formula II: 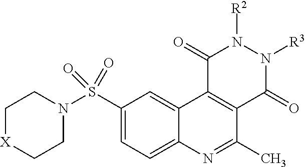
wherein “R²” and “R³” comprise at least one of:
Hydrogen;
Hydroxyl;
Alkyl group;
Alkoxy group;
Acyloxy group;
Alkenyl group;
Alkynyl group;
Aromatic group;
Heterocyclic group;
and “X” is a (0), (—CH₂—), (—O—), (CH₃N—), or an allosteric replacement,
or any salt, isomer, ester, or derivative thereof, for the modulation of caspase activity.

10. The compound, according to claim 9,
wherein “R²” or “R³” is an aromatic group comprising at least one of:
Monocyclic aromatic group or any substituted moiety thereof;
Polycyclic aromatic group or any substituted moiety thereof;
or any salt, isomer, ester, or derivative thereof, for the modulation of caspase activity.

11. The compound, according to claim 10,
wherein “R²” and “R³” are an aromatic group comprising at least one five or six membered ring or any substituted moiety thereof;
or any salt, isomer, ester, or derivative thereof, for the modulation of caspase activity.

12. The compound, according to claim 11,
wherein “R²” or “R³” is an aromatic group comprise a benzene ring, a pyridine ring, a quinoline ring, or any substituted moiety thereof;
or any salt, isomer, ester, or derivative thereof, for the modulation of caspase activity.

13. The compound, according to claim 9,
wherein “R²” or “R³” is an aromatic group comprising a fluorinated benzene ring, a fluorinated pyridine ring, a fluorinated quinoline ring, or any substituted moiety thereof;
or any salt, isomer, ester, or derivative thereof, for the modulation and diagnosis of caspase mediated diseases.

14. The compound, according to claim 1, with differential inhibitory activity against specific caspase enzymes.

15. The compound, according to claim 1, for the diagnosis and treatment of diseases that involve dysregulation of caspase activity.

16. The compound, according to claim 1, wherein the compound has an inhibitory activity against caspase 1, caspase 3 and caspase 7.

17. The compound, according to claim 1, wherein the inhibitory potential of the compound is more against caspase 7 compared to caspase 1 and caspase 3.

18. The compound, according to claim 1, wherein the compound has an IC₅₀ value of less than 100 nM against any of the caspase enzymes.

19. The compound, according to claim 1, for the treatment of diseases characterized by cell death or inflammation in acute and/or chronic conditions.

20. A pharmaceutical composition comprising any one of the compounds, according to claim 1, for the diagnosis and treatment of pathological conditions that involve upregulation of caspase activity in peripheral blood cells or other body cells individually or in any combination with other known drugs and a pharmaceutically acceptable vehicle.

21-30. (canceled)
